(19) United States
(12) Patent Application Publication
(10) Pub. No.: US 2024/0196735 A1
(43) Pub. Date: Jun. 13, 2024

(54) Organic Compound, Organic Light Emitting Diode and Organic Light Emitting Device Having the Compound

(71) Applicants: LG Display Co., Ltd., Seoul (KR); Dong-A University Research Foundation For Industry-Academy Cooperation, Busan (KR)

(72) Inventors: Dong-Ryun LEE, Paju-si (KR); Chi-Ho LEE, Paju-si (KR); Joon-Beom IM, Paju-si (KR); Han-Jin AHN, Paju-si (KR); Jun-Yun KIM, Paju-si (KR); Gi-Dong LEE, Busan (KR); Chang-Hee LEE, Busan (KR)

(21) Appl. No.: 18/495,623

(22) Filed: Oct. 26, 2023

(30) Foreign Application Priority Data
Nov. 22, 2022 (KR) 10-2022-0156940

(51) Int. Cl. H10K 85/40; C07F 7/08; C09K 11/06

(52) U.S. Cl. CPC H10K 85/40; C07F 7/0812; C09K 11/06; H10K 2101/90

(57) ABSTRACT

The present disclosure relates to an organic compound including at least one carbazolyl moiety fused with an alicyclic or hetero alicyclic ring and linked to directly or via a linker to a triazine moiety and at least one blocking moiety linked the triazine moiety, an organic light emitting diode and an organic light emitting device including the organic compound. The organic compound can implement high luminous efficiency as enhancing delayed fluorescent property, minimize reduction of the luminous lifespan caused by substituent degradation, and can secure high triplet energy level owing to no conjugation extensions. The organic light emitting diode and the organic light emitting device using the organic compound can have beneficial luminous efficiency and the luminous lifespan.

### CROSS-REFERENCE TO RELATED APPLICATIONS

This application claims the benefit of and the priority of Republic of Korea Patent Application No. 10-2022-0156940, filed in the Republic of Korea on Nov. 22, 2022, which is expressly incorporated hereby in its entirety into the present application.

### BACKGROUND

### Technical Field

The present disclosure relates to an organic compound, and more particularly to, an organic compound with beneficial luminous efficiency and luminous lifespan and an organic light emitting diode and an organic light emitting device including the compound.

### Discussion of the Related Art

A flat display device including an organic light emitting diode (OLED) has attracted attention as a display device that can replace a liquid crystal display device (LCD). The electrode configurations in the OLED can implement unidirectional or bidirectional images. Also, the OLED can be formed even on a flexible transparent substrate such as a plastic substrate so that a flexible or a foldable display device can be realized with ease using the OLED. In addition, the OLED can be driven at a lower voltage and the OLED has advantageous high color purity compared to the LCD.

Since fluorescent material uses only singlet excitons in the luminous process, the related art fluorescent material shows low luminous efficiency. On the contrary, phosphorescent material can show high luminous efficiency since it uses triplet exciton as well as singlet excitons in the luminous process. However, examples of phosphorescent material include metal complexes, which have a short luminous lifespan for commercial use. There has been a need to develop a compound with improved luminous efficiency and luminous lifespan.

### SUMMARY

Accordingly, embodiments of the present disclosure are directed to an organic compound, an organic light emitting diode and an organic light emitting device that substantially obviate one or more of the problems due to the limitations and disadvantages of the related art.

An object of the present disclosure is to provide an organic compound having beneficial luminous efficiency and excellent luminous lifespan, and an organic light emitting diode and an organic light emitting device including the compound.

Additional features and aspects will be set forth in the description that follows, and in part will be apparent from the description, or may be learned by practice of the disclosed concepts provided herein. Other features and aspects of the disclosed concept may be realized and attained by the structure particularly pointed out in the written description, or derivable therefrom, and the claims hereof as well as the appended drawings.

To achieve these and other aspects of the inventive concepts, as embodied and broadly described, in one aspect, the present disclosure provides an organic compound having the following structure of Chemical Formula 1:

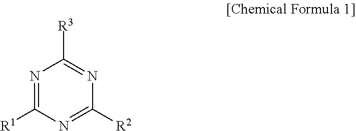

wherein, in the Chemical Formula 1,
each of R¹, R² and R³ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least one of R¹, R² and R³ has the following moiety of Chemical Formula 2 and at least one of R¹, R² and R³ has the following moiety of Chemical Formula 3:

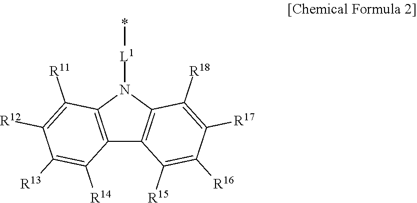

wherein, in the Chemical Formula 2,
each of R¹¹, R¹², R¹³, R¹⁴, R¹⁵, R¹⁶ and R¹⁷ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least two groups among R¹¹, R₁₂, R¹³, R¹⁴, R¹⁵, R¹⁶ and R¹⁷ are linked together to form an unsubstituted or substituted C₅-C₂₀ alicyclic ring or an unsubstituted or substituted C₃-C₂₀ hetero alicyclic ring; and
L¹ is a single bond, an unsubstituted or substituted C₆-C₃₀ arylene group or an unsubstituted or substituted C₃-C₃₀ hetero arylene group,

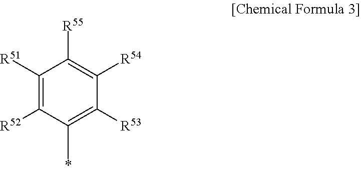

wherein, in the Chemical Formula 3,
each of R⁵¹, R⁵², R⁵³, R¹⁴ and R⁵⁵ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

For example, the moiety of Chemical Formula 2 can have the following structure of Chemical Formula 4A, Chemical Formula 4B, Chemical Formula 4C, Chemical Formula 4D or Chemical Formula 4E:

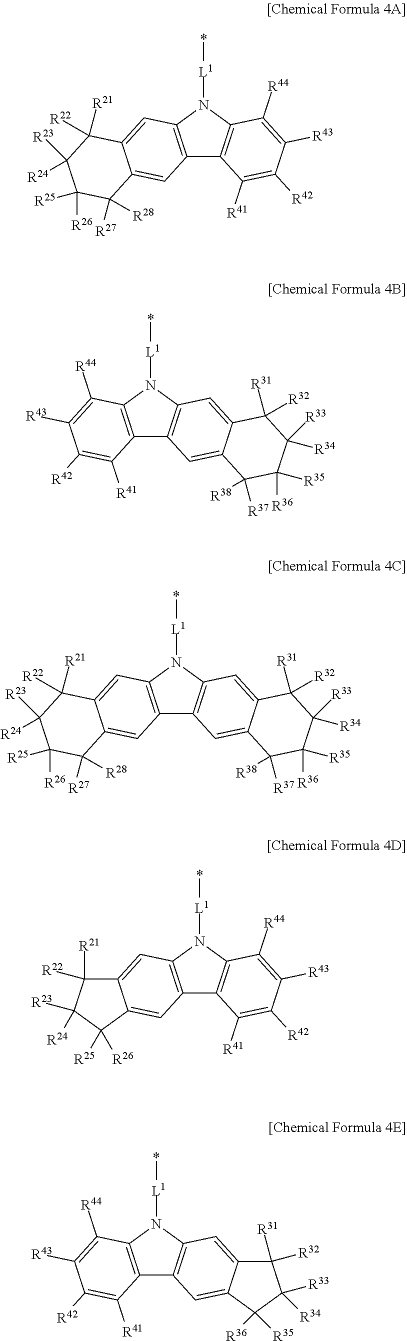

wherein, in the Chemical Formulae 4A to 4E,
L¹ is a same as defined in Chemical Formula 2;
each of R²¹, R²², R²³, R²⁴, R²⁵, R²⁶, R²⁷, R²⁸, R³¹, R³², R³³, R³⁴, R³⁵, R³⁶, R³⁷ and R³⁸ is independently hydrogen or an unsubstituted or substituted C₁-C₂₀ alkyl group; and
each of R⁴¹, R⁴², R⁴³ and R⁴⁴ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

As an example, each of R⁴¹, R⁴², R⁴³ and R⁴⁴ in Chemical Formulae 4A to 4E can be independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

In another example embodiment, the moiety of Chemical Formula 2 can have the following structure of Chemical Formula 5A, Chemical Formula 5B or Chemical Formula 5C:

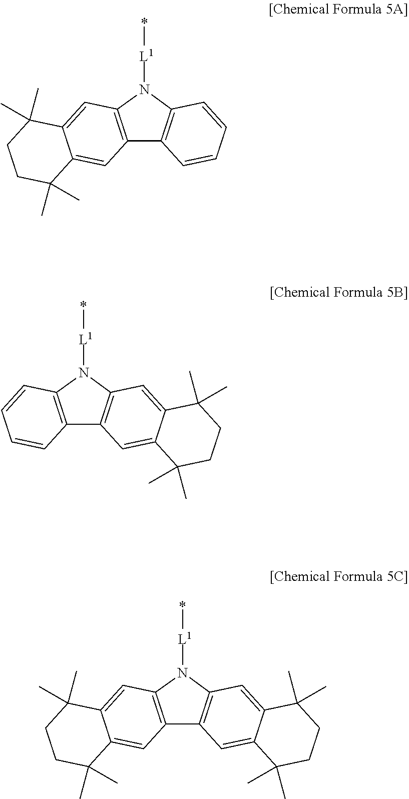

wherein, in the Chemical Formulae 5A, 5B and 5C,
L¹ is a same as defined in Chemical Formula 2.

In another example embodiment, the moiety of Chemical Formula 2 can have the following structure of Chemical Formula 6A, Chemical Formula 6B, Chemical Formula 6C, Chemical Formula 6D, Chemical Formula 6E or Chemical Formula 6F:

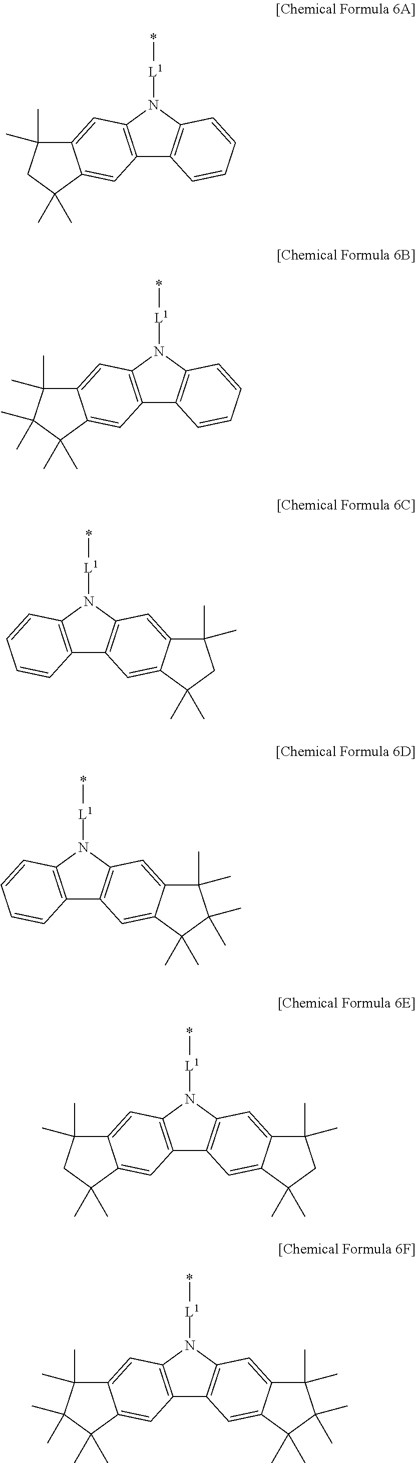

wherein, in the Chemical Formulae 6A to 6F,
L¹ is a same as defined in Chemical Formula 2.

In another example embodiment, the moiety of Chemical Formula 3 can have the following structure of Chemical Formula 7A, Chemical Formula 7B, Chemical Formula 7C or Chemical Formula 7D:

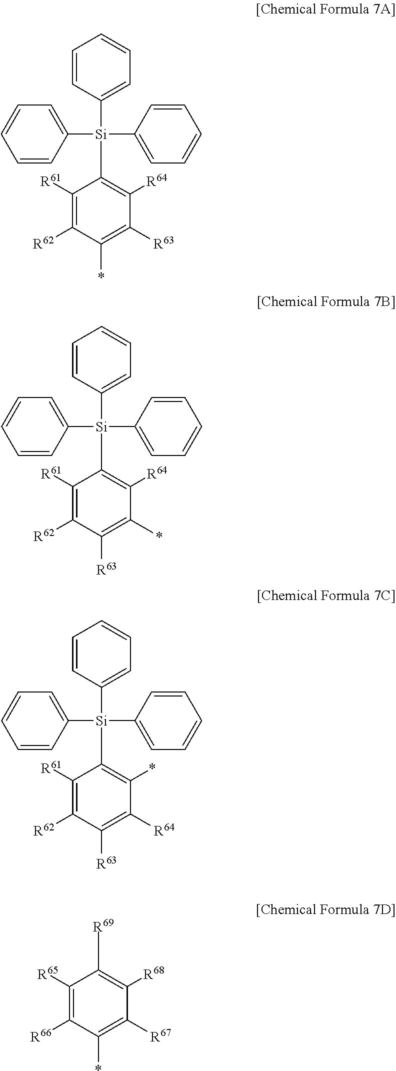

wherein, in the Chemical Formula 7A to 7D,
each of R⁶¹, R⁶², R⁶³, R⁶⁴, R⁶⁵, R⁶⁶, R⁶⁷, R⁶⁸ and R⁶⁹ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

For example, each of R⁶¹, R⁶², R⁶³, R⁶⁴, R⁶⁵, R⁶⁶, R⁶⁷, R⁶⁸ and R⁶⁹ in Chemical Formulae 7A to 7D can be independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

In another aspect, the present disclosure provides an organic light emitting diode, including: a first electrode; a second electrode facing the first electrode; and an emissive layer disposed between the first electrode and the second electrode, wherein the emissive layer includes the organic compound.

In one example embodiment, the emissive layer can include at least one emitting material layer.

As an example, the at least one emitting material layer can include a first host and the first host can include the organic compound.

In one example embodiment, the at least one emitting material layer can further include a second host.

In another example embodiment, the at least one emitting material layer can further include at least one emitter.

The at least one emitter can include at least one of a phosphorescent emitter, a fluorescent emitter and a delayed fluorescent emitter.

As an example, the at least one emitter can emit blue color light.

The emissive layer can have a single emitting unit.

Alternatively, the emissive layer can have a tandem structure of two or more emitting units.

In yet another aspect, the present disclosure provides an organic light emitting device, for example, an organic light emitting display device or an organic light emitting illumination device, includes a substrate and the organic light emitting diode over the substrate.

The organic compound includes at least one carbazolyl moiety, linked to directly or indirectly to a triazine moiety, fused with at least one alicyclic ring and/or hetero alicyclic ring. The organic compound can have beneficial delayed fluorescent property as the carbazolyl moiety has enhanced electron donor property. The organic compound can generate excitons through both ISC and RISC so that the luminous efficiency in the organic light emitting diode cannot be lowered owing to non-emissive excitons and the diode can have beneficial stability for holes leaked from other molecules.

It is possible to minimize reduction in luminescence lifetime due to thermal decomposition of other substituents linked to the carbazolyl moiety, and to prevent decrease in singlet and/or triplet energy level due to expansion of a conjugated structure.

The organic compound further includes a blocking moiety of an aromatic and/or hetero aromatic structure linked to the triazine moiety. The organic compound can maintain high triplet energy level, can have beneficial electron transporting properties and can have limited molecular packing as the exciton binding energy decrease. In addition, the organic compound has bipolar property by including the triazine moiety as an electron donor and the carbazolyl moiety as an electron acceptor.

Therefore, the organic compound can be applied to the emissive layer of an organic light emitting diode. For example, the organic compound has a higher singlet energy level S₁ and a higher triplet energy level T₁ and a wider energy bandgap between HOMO energy level and LUMO energy level compared to the emitter. Exciton energy can be rapidly transferred to the emitter in the emitting material layer by applying the organic compound as hosts of the emitting material layer. An organic light emitting diode and an organic light emitting device having greatly improved luminous efficiency and luminous lifespan can be implemented by applying the organic compound.

It is to be understood that both the foregoing general description and the following detailed description are exemplary and explanatory, and are intended to provide further explanation of the inventive concepts as claimed.

### BRIEF DESCRIPTION OF THE DRAWINGS

The accompanying drawings, which are included to provide a further understanding of the disclosure, are incorporated in and constitute a part of this application, illustrate embodiments of the disclosure and together with the description serve to explain principles of the disclosure.

FIG. 1 illustrates a schematic circuit diagram of an organic light emitting display device in accordance with the present disclosure.

FIG. 2 illustrates a cross-sectional view of an organic light emitting display device as an example of an organic light emitting device in accordance with an example embodiment of the present disclosure.

FIG. 3 illustrates a cross-sectional view of an organic light emitting diode having a single emitting part in accordance with an example embodiment of the present disclosure.

FIG. 4 illustrates a cross-sectional view of an organic light emitting diode having a single emitting part in accordance with another example embodiment of the present disclosure.

FIG. 5 illustrates a cross-sectional view of an organic light emitting display device in accordance with another example embodiment of the present disclosure.

FIG. 6 illustrates a cross-sectional view of an organic light emitting diode having a tandem structure of three emitting parts in accordance with another example embodiment of the present disclosure.

FIG. 7 illustrates emission spectra of an organic compound synthesized in accordance with the present disclosure.

### DETAILED DESCRIPTION

Reference will now be made in detail to aspects of the disclosure, examples of which are illustrated in the accompanying drawings. Wherever possible, the same reference numbers will be used throughout the drawings to refer to the same or like parts.

[Organic Compound]

Luminous materials in an emissive layer of an organic light emitting diode should have beneficial luminous properties and should not be degraded in driving the diode. An organic compound has greatly beneficial luminous properties. The organic compound can have the following structure of Chemical Formula 1:

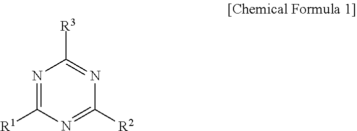

wherein, in the Chemical Formula 1,
each of R¹, R² and R³ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least one of R¹, R² and R³ has the following moiety of Chemical Formula 2 and at least one of R¹, R² and R³ has the following moiety of Chemical Formula 3:

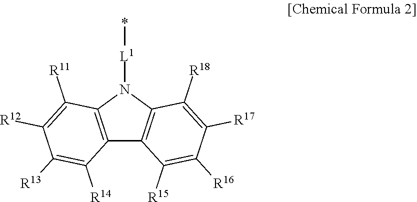

wherein, in the Chemical Formula 2,
each of R¹¹, R¹², R¹³, R¹⁴, R¹⁵, R¹⁶ and R¹⁷ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least two groups among R¹¹, R₁₂, R¹³, R¹⁴, R¹⁵, R¹⁶ and R¹⁷ are linked together to form an unsubstituted or substituted C₅-C₂₀ alicyclic ring or an unsubstituted or substituted C₃-C₂₀ hetero alicyclic ring; and
L¹ is a single bond, an unsubstituted or substituted C₆-C₃₀ arylene group or an unsubstituted or substituted C₃-C₃₀ hetero arylene group,

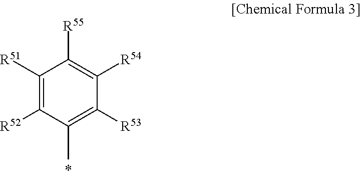

wherein, in the Chemical Formula 3,
each of R⁵¹, R⁵², R⁵³, R⁵⁴ and R⁵⁵ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

As used herein, the term “unsubstituted” means that hydrogen is directly linked to a carbon atom. “Hydrogen”, as used herein, can refer to protium.

As used herein, “substituted” means that the hydrogen is replaced with a substituent. The substituent can comprise, but is not limited to, an unsubstituted or halogen-substituted C₁-C₂₀ alkyl group, an unsubstituted or halogen-substituted C₁-C₂₀ alkoxy, halogen, a cyano group, a hydroxyl group, a carboxylic group, a carbonyl group, an amino group, a C₁-C₁₀ alkyl amino group, a C₆-C₃₀ aryl amino group, a C₃-C₃₀ hetero aryl amino group, a nitro group, a hydrazyl group, a sulfonate group, an unsubstituted or halogen-substituted C₁-C₁₀ alkyl silyl group, an unsubstituted or halogen-substituted C₁-C₁₀ alkoxy silyl group, an unsubstituted or halogen-substituted C₃-C₂₀ cyclo alkyl silyl group, an unsubstituted or halogen-substituted C₆-C₃₀ aryl silyl group, an unsubstituted or halogen-substituted C₃-C₃₀ hetero aryl silyl group, an unsubstituted or substituted C₆-C₃₀ aryl group, an unsubstituted or substituted C₃-C₃₀ hetero aryl group.

For example, each of the C₆-C₃₀ aryl group and the C₃-C₃₀ hetero aryl group can be substituted with at least one of C₁-C₂₀ alkyl, C₆-C₃₀ aryl and C₃-C₃₀ hetero aryl.

As used herein, the term “hetero” in terms such as “a hetero aromatic group”, “a hetero cyclo alkylene group”, “a hetero arylene group”, “a hetero aryl alkylene group”, “a hetero aryl oxylene group”, “a hetero cyclo alkyl group”, “a hetero aryl group”, “a hetero aryl alkyl group”, “a hetero aryl oxy group”, “a hetero aryl amino group” and the likes means that at least one carbon atom, for example 1 to 5 carbons atoms, constituting an aliphatic chain, an alicyclic group or ring or an aromatic group or ring is substituted with at least one hetero atom selected from the group consisting of N, O, S and P.

As used herein, the C₆-C₃₀ aryl group can include, but is not limited to, an unfused or fused aryl group such as phenyl, biphenyl, terphenyl, naphthyl, anthracenyl, pentalenyl, indenyl, indeno-indenyl, heptalenyl, biphenylenyl, indacenyl, phenalenyl, phenanthrenyl, benzo-phenanthrenyl, dibenzo-phenanthrenyl, azulenyl, pyrenyl, fluoranthenyl, triphenylenyl, chrysenyl, tetraphenylenyl, tetracenyl, pleiadenyl, picenyl, pentaphenylenyl, pentacenyl, fluorenyl, indeno-fluorenyl or spiro-fluorenyl. The C₆-C₃₀ arylene group can include, but is not limited to, any bivalent linking group corresponding to the above aryl group.

As used herein, the C₃-C₃₀ hetero aryl group can comprise, but is not limited to, an unfused or fused hetero aryl group such as pyrrolyl, pyridinyl, pyrimidinyl, pyrazinyl, pyridazinyl, triazinyl, tetrazinyl, imidazolyl, pyrazolyl, indolyl, iso-indolyl, indazolyl, indolizinyl, pyrrolizinyl, carbazolyl, benzo-carbazolyl, dibenzo-carbazolyl, indolo-carbazolyl, indeno-carbazolyl, benzo-furo-carbazolyl, benzo-thieno-carbazolyl, carbolinyl, quinolinyl, iso-quinolinyl, phthlazinyl, quinoxalinyl, cinnolinyl, quinazolinyl, quinolizinyl, purinyl, benzo-quinolinyl, benzo-iso-quinolinyl, benzo-quinazolinyl, benzo-quinoxalinyl, acridinyl, phenazinyl, phenoxazinyl, phenothiazinyl, phenanthrolinyl, perimidinyl, phenanthridinyl, pteridinyl, naphthyridinyl, furanyl, pyranyl, oxazinyl, oxazolyl, oxadiazolyl, triazolyl, dioxinyl, benzo-furanyl, dibenzo-furanyl, thiopyranyl, xanthenyl, chromenyl, iso-chromenyl, thioazinyl, thiophenyl, benzo-thiophenyl, dibenzo-thiophenyl, difuro-pyrazinyl, benzofuro-dibenzo-furanyl, benzothieno-benzo-thiophenyl, benzothieno-dibenzo-thiophenyl, benzothieno-benzo-furanyl, benzothieno-dibenzo-furanyl, xanthene-linked spiro acridinyl, dihydroacridinyl substituted with at least one C₁-C₁₀ alkyl and N-substituted spiro fluorenyl. The C₃-C₃₀ hetero arylene group can include, but is not limited to, any bivalent linking group corresponding to the above hetero aryl group.

As an example, each of the aromatic group (or aryl group) or the hetero aromatic group (or hetero aryl group) of R¹ to R³, R¹¹ to R¹⁸ and R⁵¹ to R⁵⁵ in Chemical Formulae 1 to 3 can consist of one to four aromatic and/or hetero aromatic rings. When the number of the aromatic and/or hetero aromatic rings of R¹ to R³, R¹¹ to R¹⁸ and R⁵¹ to R⁵⁵ becomes more than four, conjugated structure among the within the whole molecule becomes too long, thus, the organometallic compound can have too narrow energy bandgap. For example, each of the aryl group or the hetero aryl group of R¹ to R³, R¹¹ to R¹⁸ and R⁵¹ to R⁵⁵ in Chemical Formulae 1 to 3 can comprise independently, but is not limited to, phenyl, biphenyl, naphthyl, anthracenyl, pyrrolyl, triazinyl, imidazolyl, pyrazolyl, pyridinyl, pyrazinyl, pyrimidinyl, pyridazinyl, furanyl, benzo-furanyl, dibenzo-furanyl, thiophenyl, benzo-thiophenyl, dibenzo-thiophenyl, carbazolyl, acridinyl, carbolinyl, phenazinyl, phenoxazinyl, or phenothiazinyl.

The organic compound having the structure of Chemical Formula 1 includes a triazine moiety with beneficial electron transporting properties and high singlet and/or triplet energy level. The organic compound having the structure of Chemical Formula 1 includes at least one carbazolyl moiety having the structure of Chemical Formula 2.

The organic compound having the structure of Chemical Formula 1 has bipolar properties since the organic compound includes the carbazolyl moiety of an electron donor linked directly or via the linker to the triazine moiety. As the carbazolyl moiety causes holes leaked from other luminous materials to be stabilized, the organic compound can have beneficial luminous lifetime. In addition, since at least one alicyclic and/or hetero alicyclic ring is fused to the carbazolyl moiety, the carbazolyl moiety can have enhanced electron donor properties.

As delayed fluorescent properties in the entire molecule of the organic compound are improved, the organic compound has very narrow energy bandgap ΔE_{ST} between its singlet energy level S₁ and its triplet energy level T₁. Accordingly, the organic compound can implement Intersystem crossing (ISC), which is an exciton transfer mechanism from the singlet energy level S₁ to the triplet energy level T₁, as well as Reverse Intersystem Crossing (RISC), which is an exciton transfer mechanism from the triplet energy level T₁ to the singlet exciton energy level S₁, in the light emitting process. The organic compound can improve its luminous efficiency by converting upwardly non-emissive triplet excitons to its own singlet excitons. Accordingly, the luminous efficiency of the organic light emitting diode to which the organic compound is applied can be maximized or increased.

Unlike other compounds with the carbazolyl moiety where an aliphatic group is substituted, the deterioration in the luminescence lifetime due to decomposition of the functional group can be minimized in the organic compound. In addition, unlike another compound with a carbazolyl moiety where an aromatic or heteroaromatic group is substituted, the organic compound can secure high singlet and/or triplet energy levels because the conjugated structure does not expand.

In addition, the organic compound includes at least one blocking moiety having the structure of Formula 3. As described below, the moiety of Chemical Formula 3 can be an aryl silyl moiety, a hetero aryl silyl moiety, an aryl silyl germanyl moiety, or a hetero aryl silyl germanyl moiety. Organic compound including the at least one blocking moiety having the structure of Formula 3 are capable of maintaining high singlet and/or triplet energy levels. In addition, the organic compound has enhanced electron transporting properties and limited molecular packing structure so that the organic compound can implement excellent luminous efficiency and luminous lifetime.

In one example embodiment, one or two among R¹ to R³ in Chemical Formula 1 can have the structure of Chemical Formula 2 and another of R¹ to R³ in Chemical Formula 1 can have the structure of Chemical Formula 3, but is not limited thereto. Other functional groups not having the structure of Chemical Formula 2 and/or Chemical Formula 3 among R¹ to R³ in Chemical Formula 1 can be, but is not limited to, selected from the group consisting of a C₆-C₃₀ aryl group (e.g., phenyl, biphenyl or naphthyl) and a C₃-C₃₀ hetero aryl group (e.g., carbazolyl, dibenzo-furanyl or dibenzo-thiophenyl) each of which can be independently unsubstituted or substituted with at least one group of a cyano group, a C₁-C₁₀ alkyl group, a C₆-C₃₀ aryl group and/or a C₃-C₃₀ hetero aryl group.

As an example, an unsubstituted or substituted 5-membered alicyclic ring and/or an unsubstituted or substituted 6-membered alicyclic ring can be fused to the carbazole ring of the moiety in Chemical Formula 2 of the electron donor moiety. For example, one or two 5-membered alicyclic ring and/or 6-membered alicyclic ring can be fused to the carbazole ring, but is not limited thereto. The electron donor moiety with such a molecular conformation can have, but is not limited to, the following structure of Chemical Formula 4A, Chemical Formula 4B, Chemical Formula 4C, Chemical Formula 4D or Chemical Formula 4E:

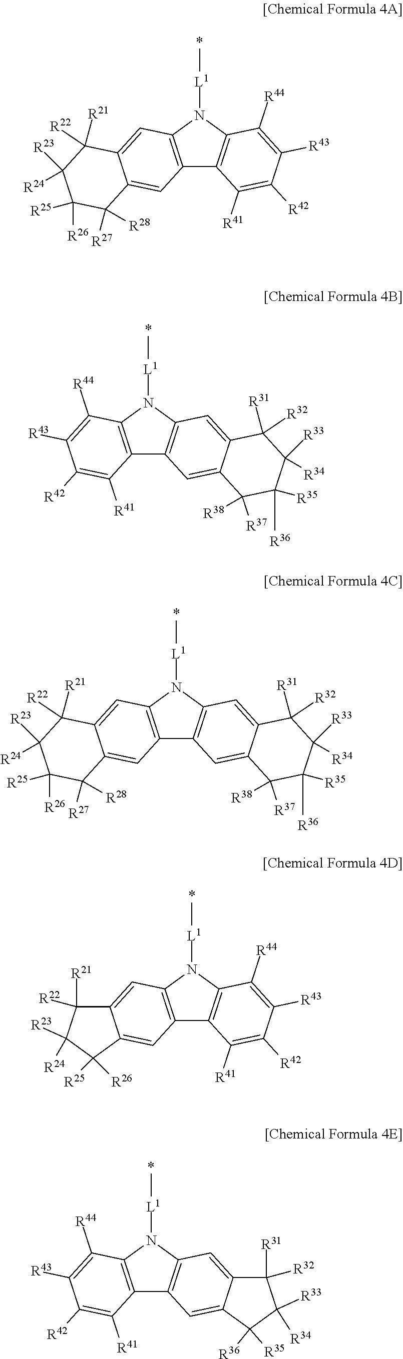

wherein, in the Chemical Formulae 4A to 4E,
L¹ is a same as defined in Chemical Formula 2;
each of R²¹, R²², R²³, R²⁴, R²⁵, R²⁶, R²⁷, R²⁸, R³¹, R³², R³³, R³⁴, R³⁵, R³⁶, R³⁷ and R³⁸ is independently hydrogen or an unsubstituted or substituted C₁-C₂₀ alkyl group; and
each of R⁴¹, R⁴², R⁴³ and R⁴⁴ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

In one example embodiment, each of R⁴¹, R⁴², R⁴³ and R⁴⁴ in Chemical Formulae 4A to 4E can be independently hydrogen, an unsubstituted or substituted C₁-C₂₀ alkyl group, an unsubstituted or substituted C₆-C₃₀ aryl group or an unsubstituted or substituted C₃-C₃₀ hetero aryl group, for example, hydrogen or an unsubstituted or substituted C₁-C₂₀ alkyl group.

For example, the 6-membered alicyclic ring fused to the carbazole ring in Chemical Formulae 4A to 4E can be unsubstitued or substituted with a C₁-C₁₀ alkyl group. The electron donor moiety with such a molecular conformation can have, but is not limited to, the following structure of Chemical Formula 5A, Chemical Formula 5B or Chemical Formula 5C:

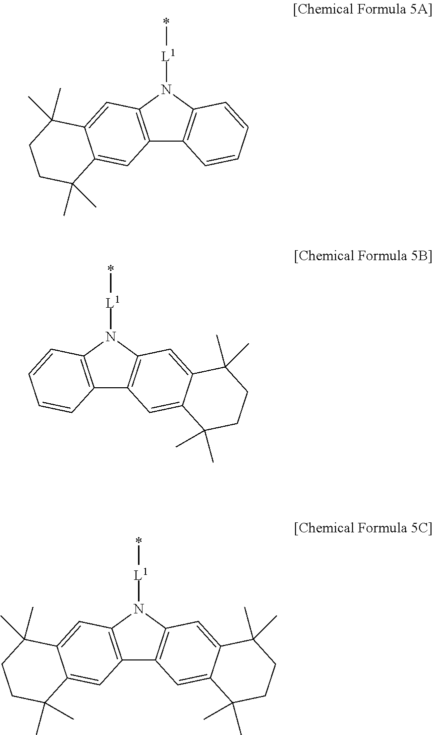

wherein, in the Chemical Formulae 5A, 5B and 5C,
L¹ is a same as defined in Chemical Formula 2.

In another example embodiment, the 5-membered alicyclic ring fused to the carbazole ring in Chemical Formulae 4A to 4E can be unsubstitued or substituted with a C₁-C₁₀ alkyl group. The electron donor moiety with such a molecular conformation can have, but is not limited to, the following structure of Chemical Formula 6A, Chemical Formula 6B, Chemical Formula 6C, Chemical Formula 6D, Chemical Formula 6E or Chemical Formula 6F:

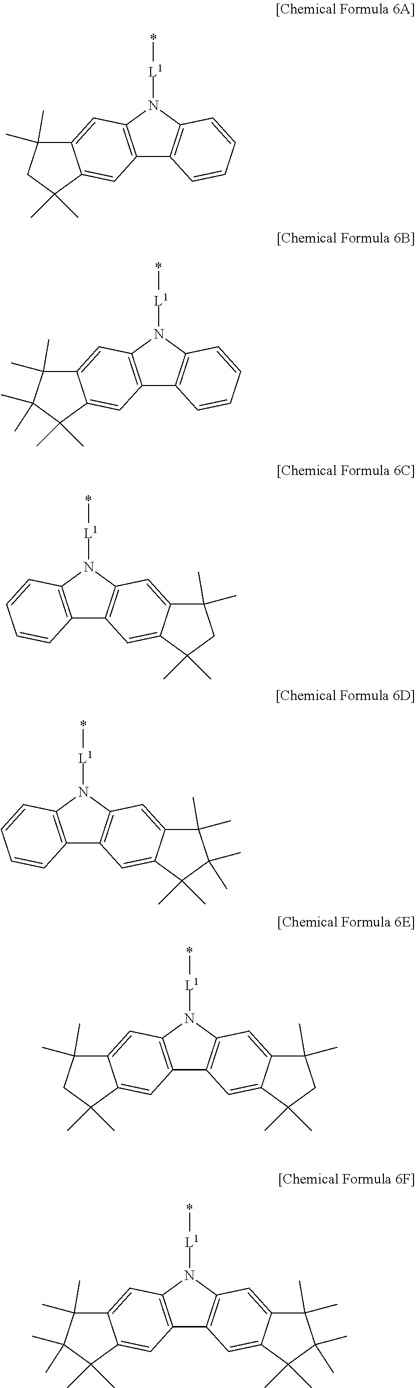

wherein, in the Chemical Formulae 6A to 6F,
L¹ is a same as defined in Chemical Formula 2.

The moiety of Chemical Formula 3 of the blocking moiety can include, but is not limited to, an unsubstitued or substituted tri-aryl silyl moiety or an unsubstituted or substituted phenyl moiety linked to the triazine moiety of the electron acceptor moiety via a benzene ring. The blocking moiety with such a molecular conformation can have, but is not limited to, the following Chemical Formula 7A, Chemical Formula 7B, Chemical Formula 7C or Chemical Formula 7D:

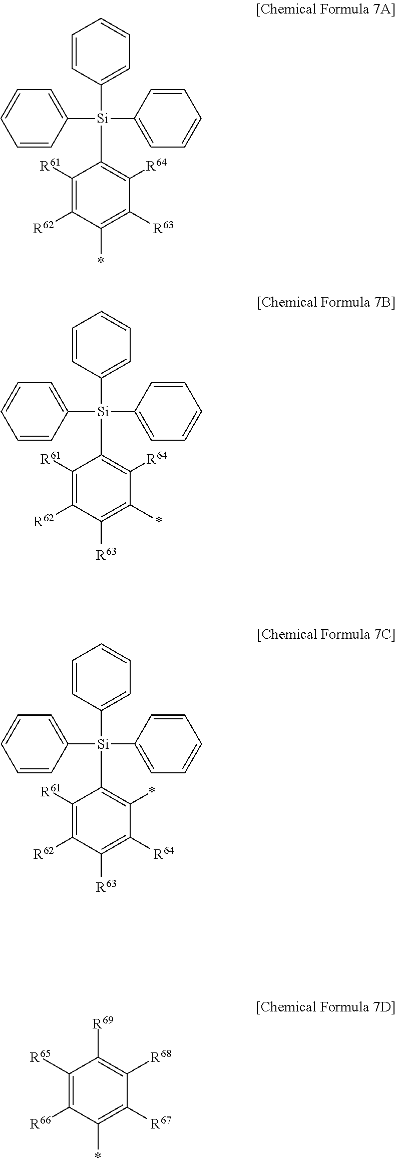

wherein, in the Chemical Formula 7A to 7D,
each of R⁶¹, R⁶², R⁶³, R⁶⁴, R⁶⁵, R⁶⁶, R⁶⁷, R⁶⁸ and R⁶⁹ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

For example, each of R⁶¹, R⁶², R⁶³, R⁶⁴, R⁶⁵, R⁶⁶, R⁶⁷, R⁶⁸ and R⁶⁹ in Chemical Formulae 7A to 7D can be independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

More particularly, the organic compound having the structure of Chemical Formulae 1 to 7F can be, but is not limited to, at least one of the following compounds of Chemical Formula 8:

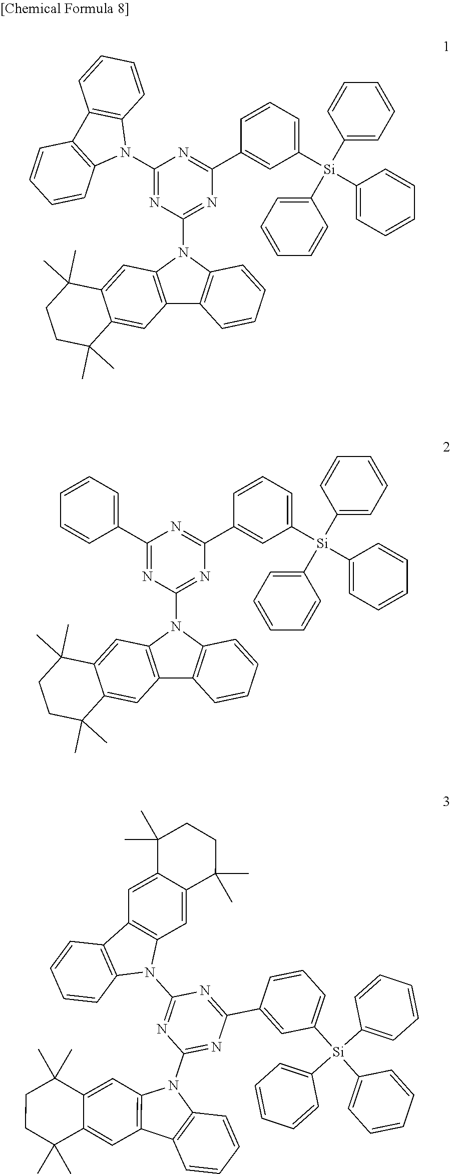 [SMILES: CC1(C)CCC(C)(C)c2cc3c(cc21)c1ccccc1n3-c1nc(-c2cccc([Si](c3ccccc3)(c3ccccc3)c3ccccc3)c2)nc(-n2c3ccccc3c3ccccc32)n1 ; CC1(C)CCC(C)(C)c2cc3c(cc21)c1ccccc1n3-c1nc(-c2ccccc2)nc(-c2cccc([Si](c3ccccc3)(c3ccccc3)c3ccccc3)c2)n1 ; CC1(C)CCC(C)(C)c2cc3c(cc21)c1ccccc1n3-c1nc(-c2cccc([Si](c3ccccc3)(c3ccccc3)c3ccccc3)c2)nc(-n2c3ccccc3c3cc4c(cc32)C(C)(C)CCC4(C)C)n1]

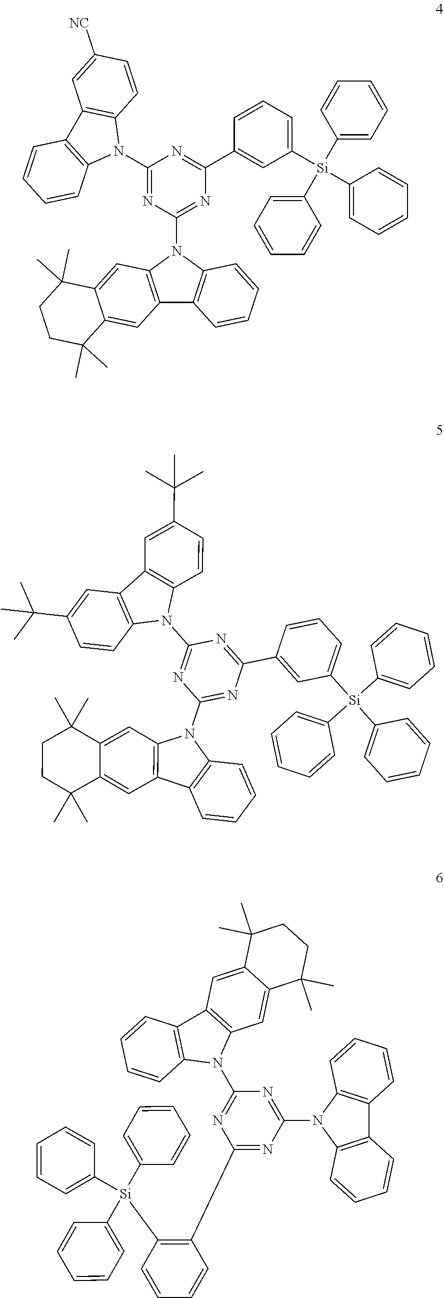 [SMILES: CC1(C)CCC(C)(C)c2cc3c(cc21)c1ccccc1n3-c1nc(-c2cccc([Si](c3ccccc3)(c3ccccc3)c3ccccc3)c2)nc(-n2c3ccccc3c3cc(C#N)ccc32)n1 ; CC(C)(C)c1ccc2c(c1)c1cc(C(C)(C)C)ccc1n2-c1nc(-c2cccc([Si](c3ccccc3)(c3ccccc3)c3ccccc3)c2)nc(-n2c3ccccc3c3cc4c(cc32)C(C)(C)CCC4(C)C)n1 ; CC1(C)CCC(C)(C)c2cc3c(cc21)c1ccccc1n3-c1nc(-c2ccccc2[Si](c2ccccc2)(c2ccccc2)c2ccccc2)nc(-n2c3ccccc3c3ccccc32)n1]

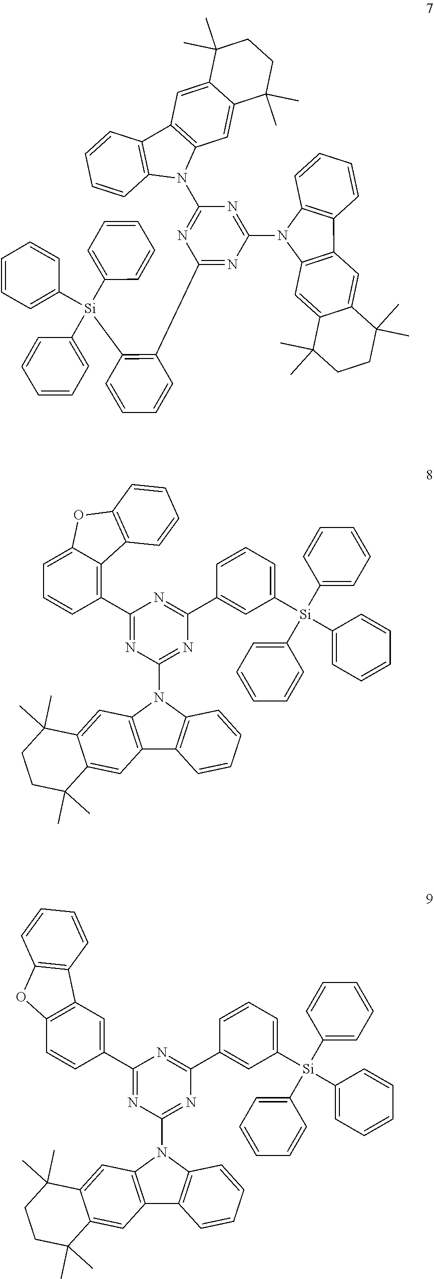

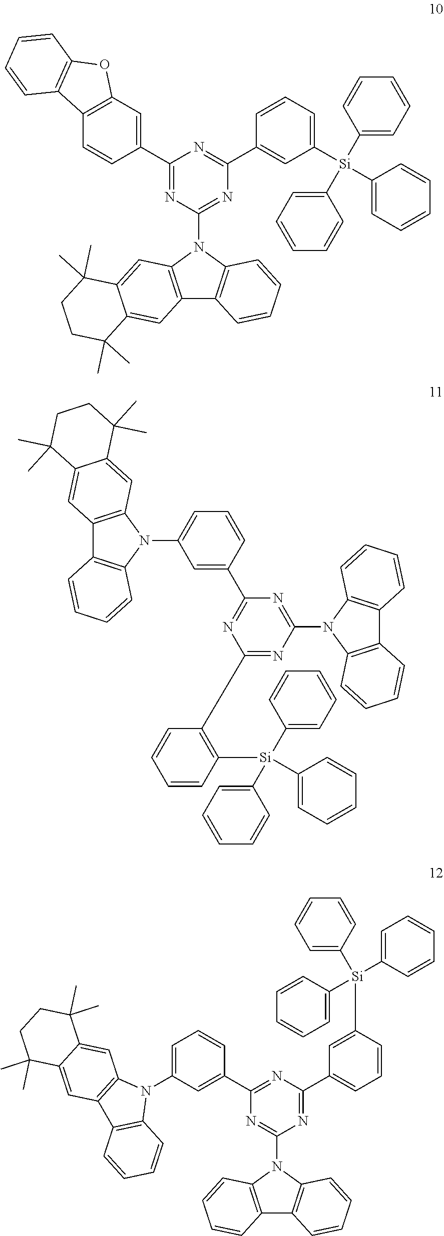

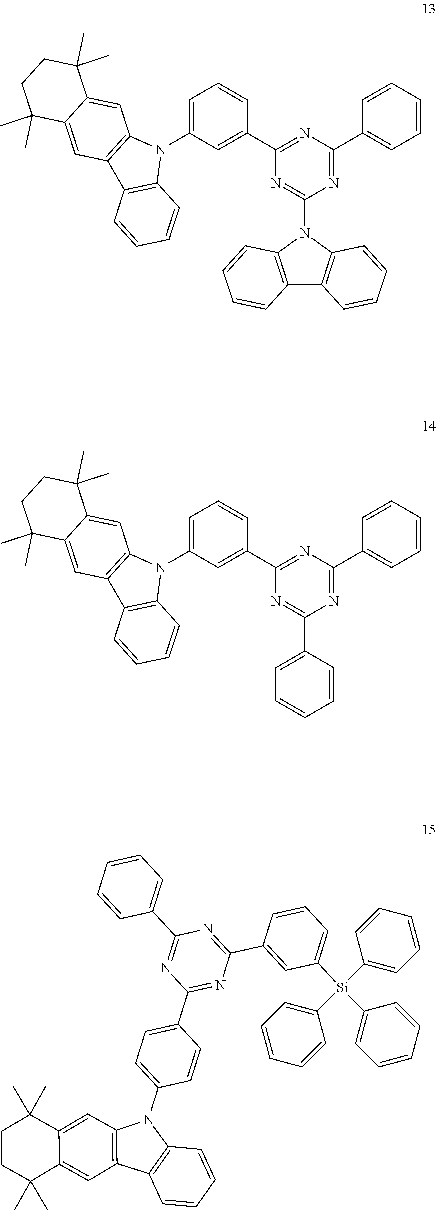 [SMILES: CC1(C)CCC(C)(C)c2cc3c(cc21)c1ccccc1n3-c1cccc(-c2nc(-c3ccccc3)nc(-n3c4ccccc4c4ccccc43)n2)c1 ; CC1(C)CCC(C)(C)c2cc3c(cc21)c1ccccc1n3-c1cccc(-c2nc(-c3ccccc3)nc(-c3ccccc3)n2)c1 ; CC1(C)CCC(C)(C)c2cc3c(cc21)c1ccccc1n3-c1ccc(-c2nc(-c3ccccc3)nc(-c3cccc([Si](c4ccccc4)(c4ccccc4)c4ccccc4)c3)n2)cc1]

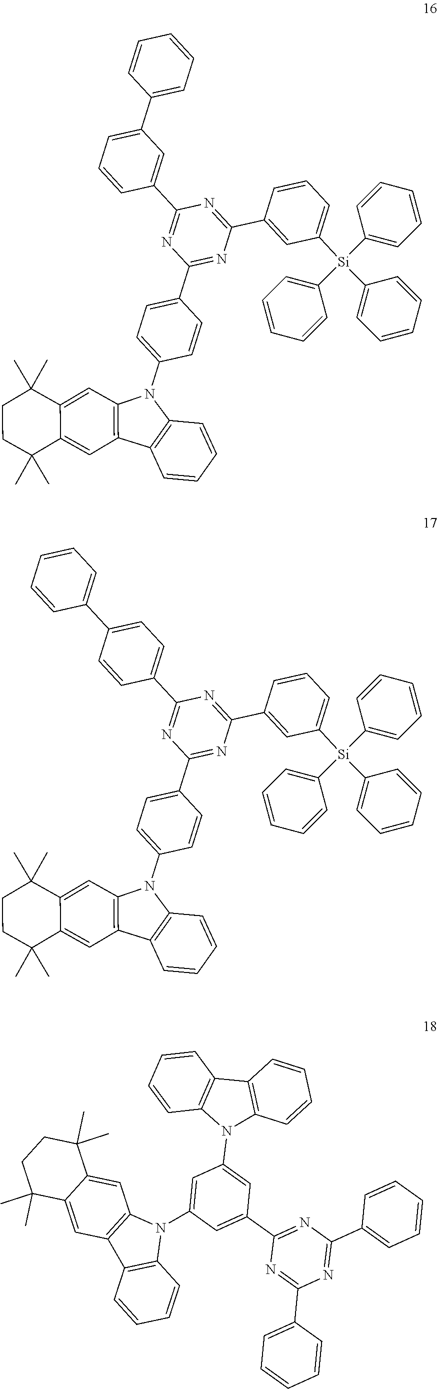 [SMILES: CC1(C)CCC(C)(C)c2cc3c(cc21)c1ccccc1n3-c1ccc(-c2nc(-c3cccc(-c4ccccc4)c3)nc(-c3cccc([Si](c4ccccc4)(c4ccccc4)c4ccccc4)c3)n2)cc1 ; CC1(C)CCC(C)(C)c2cc3c(cc21)c1ccccc1n3-c1ccc(-c2nc(-c3ccc(-c4ccccc4)cc3)nc(-c3cccc([Si](c4ccccc4)(c4ccccc4)c4ccccc4)c3)n2)cc1 ; CC1(C)CCC(C)(C)c2cc3c(cc21)c1ccccc1n3-c1cc(-c2nc(-c3ccccc3)nc(-c3ccccc3)n2)cc(-n2c3ccccc3c3ccccc32)c1]

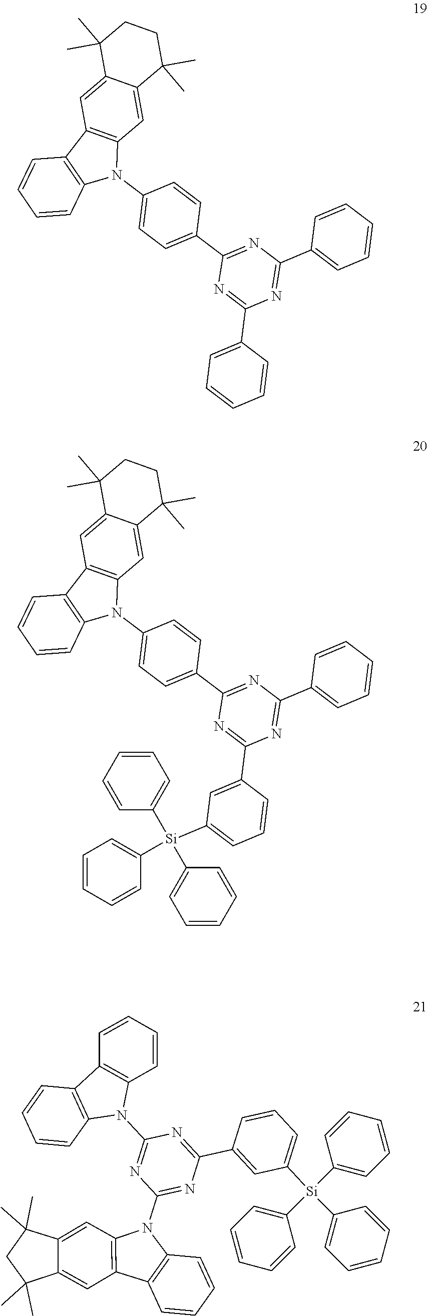 [SMILES: CC1(C)CCC(C)(C)c2cc3c(cc21)c1ccccc1n3-c1ccc(-c2nc(-c3ccccc3)nc(-c3ccccc3)n2)cc1 ; CC1(C)CCC(C)(C)c2cc3c(cc21)c1ccccc1n3-c1ccc(-c2nc(-c3ccccc3)nc(-c3cccc([Si](c4ccccc4)(c4ccccc4)c4ccccc4)c3)n2)cc1 ; CC1(C)CC(C)(C)c2cc3c(cc21)c1ccccc1n3-c1nc(-c2cccc([Si](c3ccccc3)(c3ccccc3)c3ccccc3)c2)nc(-n2c3ccccc3c3ccccc32)n1]

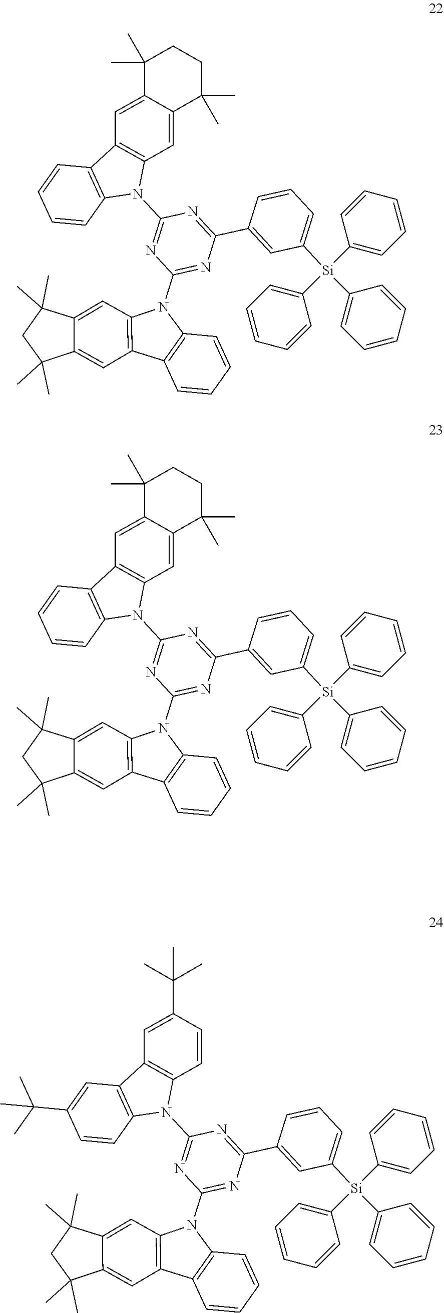 [SMILES: CC1(C)CCC(C)(C)c2cc3c(cc21)c1ccccc1n3-c1nc(-c2cccc([Si](c3ccccc3)(c3ccccc3)c3ccccc3)c2)nc(-n2c3ccccc3c3cc4c(cc32)C(C)(C)CC4(C)C)n1 ; CC1(C)CCC(C)(C)c2cc3c(cc21)c1ccccc1n3-c1nc(-c2cccc([Si](c3ccccc3)(c3ccccc3)c3ccccc3)c2)nc(-n2c3ccccc3c3cc4c(cc32)C(C)(C)CC4(C)C)n1 ; CC(C)(C)c1ccc2c(c1)c1cc(C(C)(C)C)ccc1n2-c1nc(-c2cccc([Si](c3ccccc3)(c3ccccc3)c3ccccc3)c2)nc(-n2c3ccccc3c3cc4c(cc32)C(C)(C)CC4(C)C)n1]

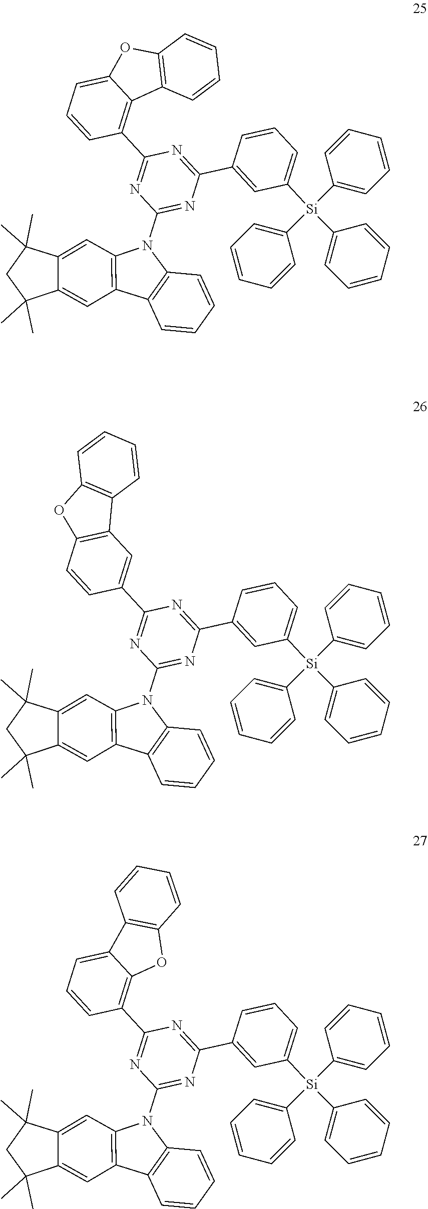 [SMILES: CC1(C)CC(C)(C)c2cc3c(cc21)c1ccccc1n3-c1nc(-c2cccc([Si](c3ccccc3)(c3ccccc3)c3ccccc3)c2)nc(-c2cccc3oc4ccccc4c23)n1 ; CC1(C)CC(C)(C)c2cc3c(cc21)c1ccccc1n3-c1nc(-c2cccc([Si](c3ccccc3)(c3ccccc3)c3ccccc3)c2)nc(-c2ccc3oc4ccccc4c3c2)n1 ; CC1(C)CC(C)(C)c2cc3c(cc21)c1ccccc1n3-c1nc(-c2cccc([Si](c3ccccc3)(c3ccccc3)c3ccccc3)c2)nc(-c2cccc3c2oc2ccccc23)n1]

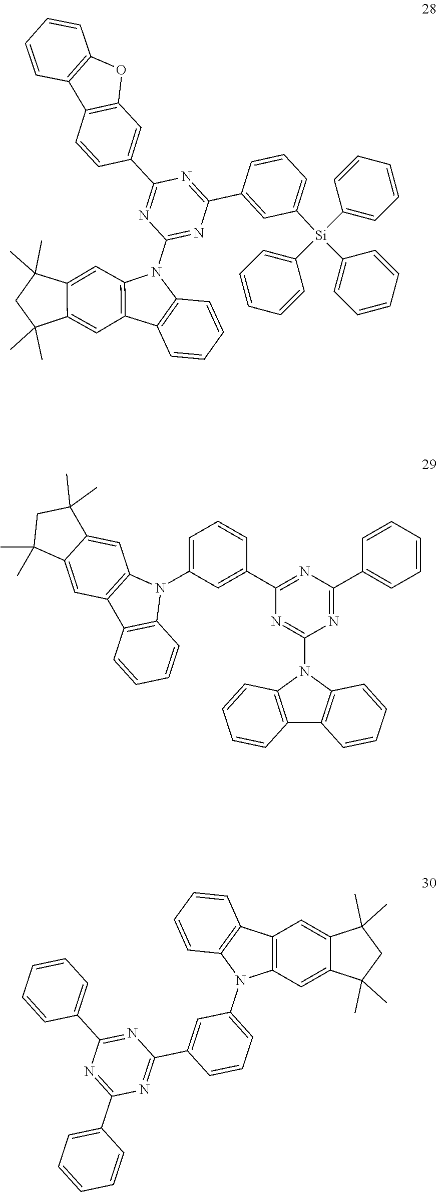 [SMILES: CC1(C)CC(C)(C)c2cc3c(cc21)c1ccccc1n3-c1nc(-c2cccc([Si](c3ccccc3)(c3ccccc3)c3ccccc3)c2)nc(-c2ccc3c(c2)oc2ccccc23)n1 ; CC1(C)CC(C)(C)c2cc3c(cc21)c1ccccc1n3-c1cccc(-c2nc(-c3ccccc3)nc(-n3c4ccccc4c4ccccc43)n2)c1 ; CC1(C)CC(C)(C)c2cc3c(cc21)c1ccccc1n3-c1cccc(-c2nc(-c3ccccc3)nc(-c3ccccc3)n2)c1]

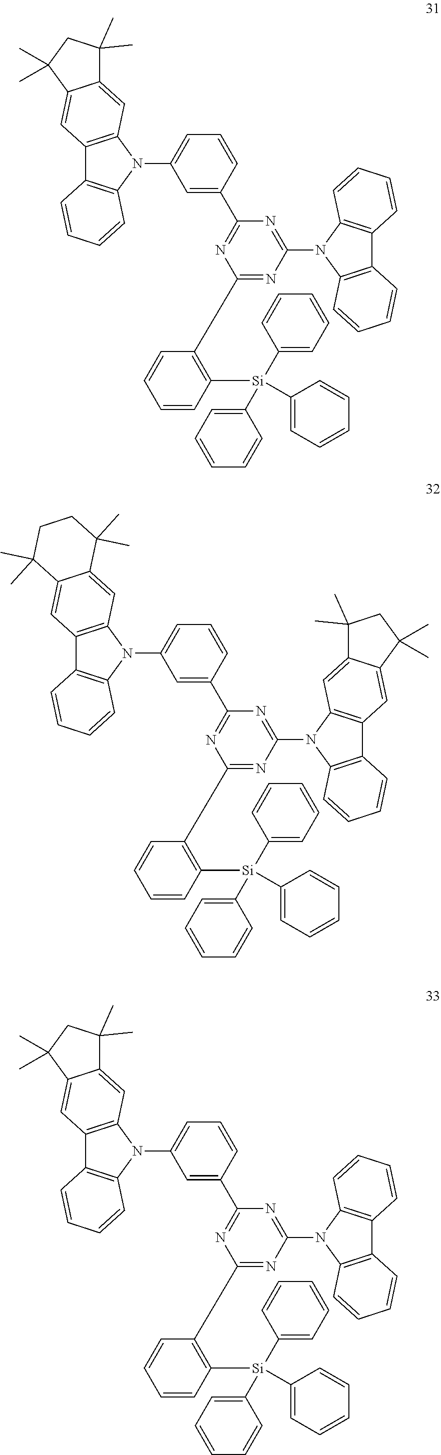

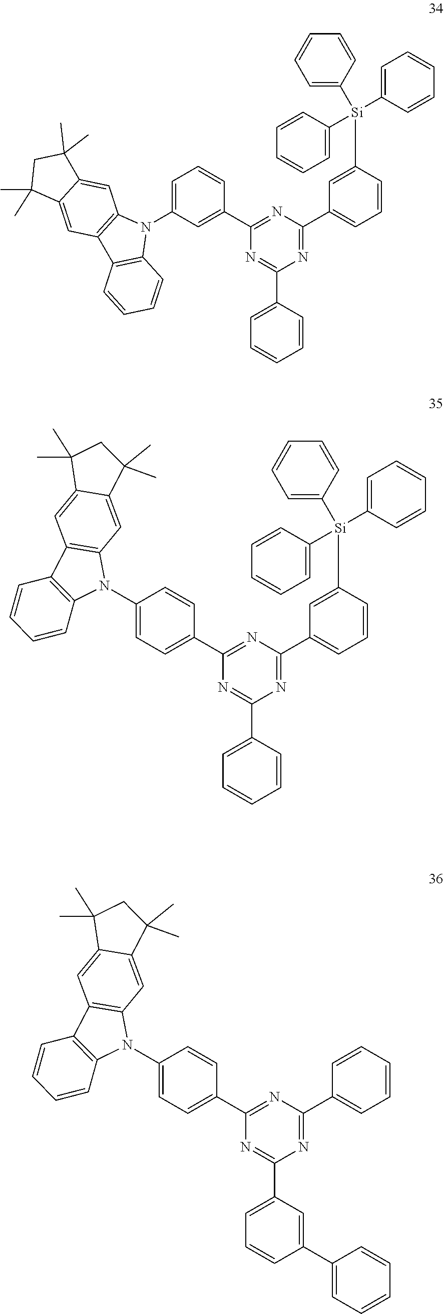 [SMILES: CC1(C)CC(C)(C)c2cc3c(cc21)c1ccccc1n3-c1cccc(-c2nc(-c3ccccc3)nc(-c3cccc([Si](c4ccccc4)(c4ccccc4)c4ccccc4)c3)n2)c1 ; CC1(C)CC(C)(C)c2cc3c(cc21)c1ccccc1n3-c1ccc(-c2nc(-c3ccccc3)nc(-c3cccc([Si](c4ccccc4)(c4ccccc4)c4ccccc4)c3)n2)cc1 ; CC1(C)CC(C)(C)c2cc3c(cc21)c1ccccc1n3-c1ccc(-c2nc(-c3ccccc3)nc(-c3cccc(-c4ccccc4)c3)n2)cc1]

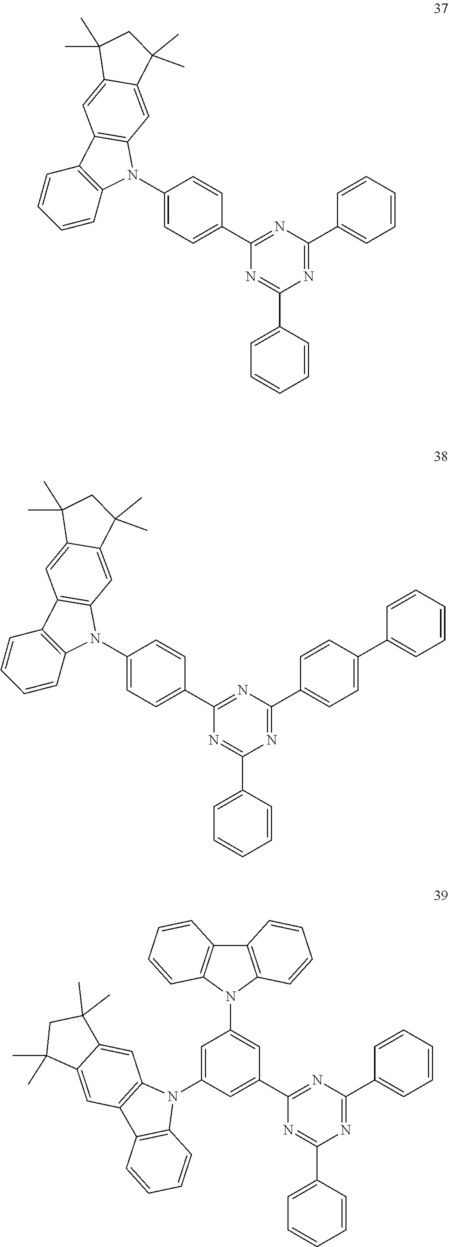 [SMILES: CC1(C)CC(C)(C)c2cc3c(cc21)c1ccccc1n3-c1ccc(-c2nc(-c3ccccc3)nc(-c3ccccc3)n2)cc1 ; CC1(C)CC(C)(C)c2cc3c(cc21)c1ccccc1n3-c1ccc(-c2nc(-c3ccccc3)nc(-c3ccc(-c4ccccc4)cc3)n2)cc1 ; CC1(C)CC(C)(C)c2cc3c(cc21)c1ccccc1n3-c1cc(-c2nc(-c3ccccc3)nc(-c3ccccc3)n2)cc(-n2c3ccccc3c3ccccc32)c1]

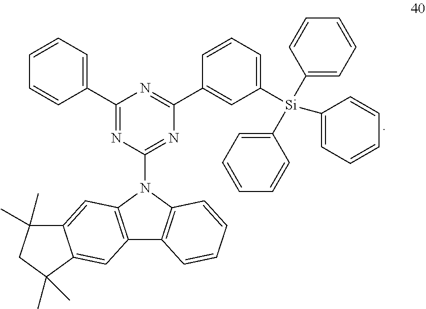 [SMILES: CC1(C)CC(C)(C)c2cc3c(cc21)c1ccccc1n3-c1nc(-c2ccccc2)nc(-c2cccc([Si](c3ccccc3)(c3ccccc3)c3ccccc3)c2)n1]

The organic compound having the structure of Chemical Formulae 1 to 8 includes the triazine moiety of the electron acceptor moiety and at least one carbazolyl moiety of the electron donor moiety so that the organic compound can accept rapidly holes as well as electrons. The organic compound has improved delayed fluorescent property, beneficial luminous efficiency and excellent stability for holes. The organic compound can prevent luminous lifetime from reducing owing to thermal dissociation of substituents and can maintain higher triplet and/or singlet energy levels. In addition, the organic compound has wider energy bandgap between the HOMO energy level and the LUMO energy level compared to the emitter. Accordingly, the organic light emitting diode where the organic compound is applied to a common layer such as a hole injection layer, a hole transport layer, an electron blocking layer, a hole blocking layer and an electron transport layer, or to a host in an emitting material layer can have beneficial luminous efficiency and luminous lifetime.

### [Organic Light Emitting Diode and Organic Light Emitting Device]

The luminous efficiency and/or the luminous lifespan of an organic light emitting diode where the organic compound having the structure of Chemical Formulae 1 to 8 is applied to an emissive layer can be improved. As an example, the emissive layer including the organic compound having the structure of Chemical Formulae 1 to 8 can be applied to an organic light emitting diode with a single emitting part in a red pixel region, a green pixel region and/or a blue pixel region. Alternatively, the emissive layer including the organic compound having the structure of Chemical Formulae 1 to 8 can be applied to an organic light emitting diode of having a tandem structure where at least two emitting parts are stacked.

The organic light emitting diode where an emissive layer includes the organic compound having the structure of Chemical Formulae 1 to 8 can be applied to an organic light emitting device such as an organic light emitting display device or an organic light emitting illumination device. As an example, an organic light emitting display device will be described.

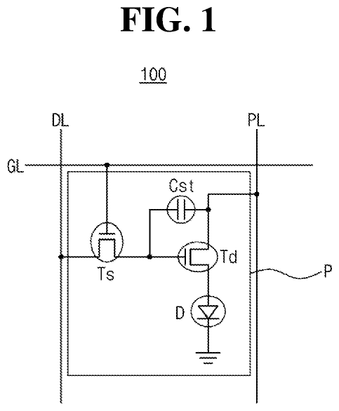

FIG. 1 illustrates a schematic circuit diagram of an organic light emitting display device in accordance with the present disclosure. As illustrated in FIG. 1, a gate line GL, a data line DL and power line PL, each of which crosses each other to define a pixel region P, in an organic light emitting display device 100. A switching thin film transistor Ts, a driving thin film transistor Td, a storage capacitor Cst and an organic light emitting diode D are disposed within the pixel region P. The pixel region P may include a red (R) pixel region, a green (G) pixel region and a blue (B) pixel region. However, embodiments of the present disclosure are not limited to such examples.

The switching thin film transistor Ts is connected to the gate line GL and the data line DL. The driving thin film transistor Td and the storage capacitor Cst are connected between the switching thin film transistor Ts and the power line PL. The organic light emitting diode D is connected to the driving thin film transistor Td. When the switching thin film transistor Ts is turned on by a gate signal applied to the gate line GL, a data signal applied to the data line DL is applied to a gate electrode of the driving thin film transistor Td and one electrode of the storage capacitor Cst through the switching thin film transistor Ts.

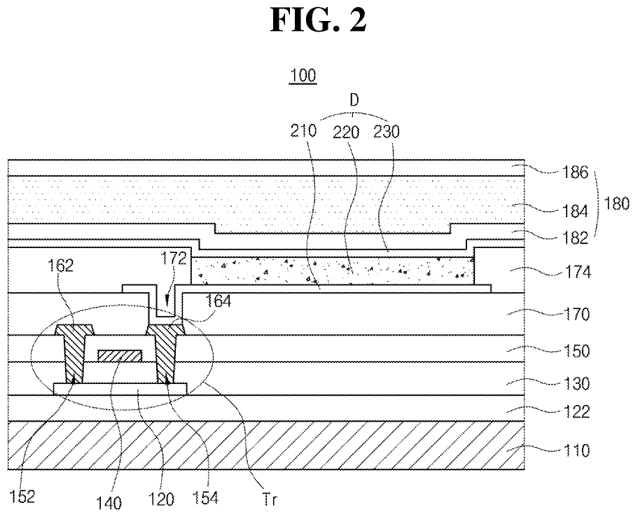

The driving thin film transistor Td is turned on by the data signal applied to the gate electrode 140 (FIG. 2) so that a current proportional to the data signal is supplied from the power line PL to the organic light emitting diode D through the driving thin film transistor Td. And then, the organic light emitting diode D emits light having a luminance proportional to the current flowing through the driving thin film transistor Td. In this case, the storage capacitor Cst is charged with a voltage proportional to the data signal so that the voltage of the gate electrode in the driving thin film transistor Td is kept constant during one frame. Therefore, the organic light emitting display device can display a desired image.

FIG. 2 illustrates a schematic cross-sectional view of an organic light emitting display device in accordance with an example embodiment of the present disclosure. As illustrated in FIG. 2, the organic light emitting display device 100 includes a substrate 110, a thin-film transistor Tr on the substrate 110, and an organic light emitting diode D connected to the thin film transistor Tr.

As an example, the substrate 110 can include a red pixel region, a green pixel region and a blue pixel region and an organic light emitting diode D can be located in each pixel region. Each of the organic light emitting diodes D emitting red, green and blue light, respectively, is located correspondingly in the red pixel region, the green pixel region and the blue pixel region.

The substrate 110 can include, but is not limited to, glass, thin flexible material and/or polymer plastics. For example, the flexible material can be selected from the group, but is not limited to, polyimide (PI), polyethersulfone (PES), polyethylenenaphthalate (PEN), polyethylene terephthalate (PET), polycarbonate (PC) and/or combinations thereof. The substrate 110, on which the thin film transistor Tr and the organic light emitting diode D are arranged, forms an array substrate.

A buffer layer 122 can be disposed on the substrate 110. The thin film transistor Tr can be disposed on the buffer layer 122. The buffer layer 122 can be omitted.

A semiconductor layer 120 is disposed on the buffer layer 122. In one example embodiment, the semiconductor layer 120 can include, but is not limited to, oxide semiconductor materials. In this case, a light-shield pattern may be disposed under the semiconductor layer 120, and the light-shield pattern can prevent light from being incident toward the semiconductor layer 120, and thereby, preventing or reducing the semiconductor layer 120 from being degraded by the light. Alternatively, the semiconductor layer 120 can include polycrystalline silicon. In this case, opposite edges of the semiconductor layer 120 can be doped with impurities.

A gate insulating layer 130 including an insulating material is disposed on the semiconductor layer 120. The gate insulating layer 130 can include, but is not limited to, an inorganic insulating material such as silicon oxide (SiOₓ, wherein 0<x≤2) or silicon nitride (SiNₓ, wherein 0<x≤2).

A gate electrode 140 made of a conductive material such as a metal is disposed on the gate insulating layer 130 so as to correspond to a center of the semiconductor layer 120. While the gate insulating layer 130 is disposed on a whole area of the substrate 110 as shown in FIG. 2, the gate insulating layer 130 can be patterned identically as the gate electrode 140.

An interlayer insulating layer 150 including an insulating material is disposed on the gate electrode 140 with and covers an entire surface of the substrate 110. The interlayer insulating layer 150 can include, but is not limited to, an inorganic insulating material such as silicon oxide (SiOₓ, wherein 0<x≤2) or silicon nitride (SiNₓ, wherein 0<x≤2), or an organic insulating material such as benzocyclobutene or photo-acryl.

The interlayer insulating layer 150 has a first semiconductor layer contact hole 152 and a second semiconductor layer contact hole 154 that expose or do not cover a portion of the surface nearer to the opposing ends than to a center of the semiconductor layer 120. The first and second semiconductor layer contact holes 152 and 154 are disposed on opposite sides of the gate electrode 140 and spaced apart from the gate electrode 140. The first and second semiconductor layer contact holes 152 and 154 are formed within the gate insulating layer 130 in FIG. 2. Alternatively, the first and second semiconductor layer contact holes 152 and 154 can be formed only within the interlayer insulating layer 150 when the gate insulating layer 130 is patterned identically as the gate electrode 140.

A source electrode 162 and a drain electrode 164, which are made of conductive material such as a metal, are disposed on the interlayer insulating layer 150. The source electrode 162 and the drain electrode 164 are spaced apart from each other on opposing sides of the gate electrode 140, and contact both sides of the semiconductor layer 120 through the first and second semiconductor layer contact holes 152 and 154, respectively.

The semiconductor layer 120, the gate electrode 140, the source electrode 162 and the drain electrode 164 constitute the thin film transistor Tr, which acts as a driving element. The thin film transistor Tr in FIG. 2 has a coplanar structure in which the gate electrode 140, the source electrode 162 and the drain electrode 164 are disposed on the semiconductor layer 120. Alternatively, the thin film transistor Tr can have an inverted staggered structure in which a gate electrode is disposed under a semiconductor layer and a source and drain electrodes are disposed on the semiconductor layer. In this case, the semiconductor layer can include amorphous silicon.

The gate line GL and the data line DL, which cross each other to define a pixel region P, and a switching element Ts, which is connected to the gate line GL and the data line DL, can be further formed in the pixel region P. The switching element Ts is connected to the thin film transistor Tr, which is a driving element. In addition, the power line PL is spaced apart in parallel from the gate line GL or the data line DL. The thin film transistor Tr may further include a storage capacitor Cst configured to constantly keep a voltage of the gate electrode 140 for one frame.

A passivation layer 170 is disposed on the source and drain electrodes 162 and 164. The passivation layer 170 covers the thin film transistor Tr on the whole substrate 110. The passivation layer 170 has a flat top surface and a drain contact hole 172 that exposes or does not cover the drain electrode 164 of the thin film transistor Tr. While the drain contact hole 172 is disposed on the second semiconductor layer contact hole 154, it may be spaced apart from the second semiconductor layer contact hole 154.

The organic light emitting diode (OLED) D includes a first electrode 210 that is disposed on the passivation layer 170 and connected to the drain electrode 164 of the thin film transistor Tr. The OLED D further includes an emissive layer 220 and a second electrode 230 each of which is disposed sequentially on the first electrode 210.

The first electrode 210 is disposed separately in each pixel region. The first electrode 210 can be an anode and include conductive material having relatively high work function value. For example, the first electrode 210 can include a transparent conductive oxide (TCO). More particularly, the first electrode 210 can include, but is not limited to, indium tin oxide (ITO), indium zinc oxide (IZO), indium tin zinc oxide (ITZO), tin oxide (SnO), zinc oxide (ZnO), indium cerium oxide (ICO), aluminum doped zinc oxide (AZO), and/or combinations thereof.

In one example embodiment, when the organic light emitting display device 100 is a bottom-emission type, the first electrode 210 can have a single-layered structure of the TCO. Alternatively, when the organic light emitting display device 100 is a top-emission type, a reflective electrode or a reflective layer may be disposed under the first electrode 210. For example, the reflective electrode or the reflective layer can include, but is not limited to, silver (Ag) or aluminum-palladium-copper (APC) alloy. In the OLED D of the top-emission type, the first electrode 210 can have a triple-layered structure of ITO/Ag/ITO or ITO/APC/ITO.

In addition, a bank layer 174 is disposed on the passivation layer 170 in order to cover edges of the first electrode 210. The bank layer 174 exposes or does not cover a center of the first electrode 210 corresponding to each pixel region. The bank layer 174 can be omitted.

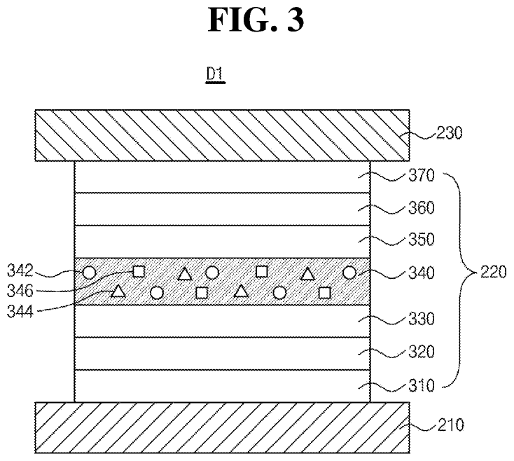

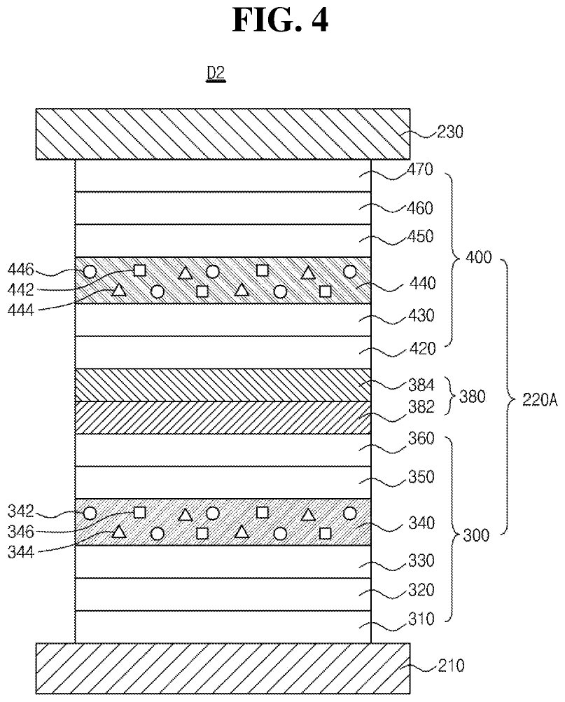

An emissive layer 220 is disposed on the first electrode 210. In one example embodiment, the emissive layer 220 can have a single-layered structure of an emitting material layer (EML). Alternatively, the emissive layer 220 can have a multiple-layered structure of a hole injection layer (HIL), a hole transport layer (HTL), an electron blocking layer (EBL), an EML, a hole blocking layer (HBL), an electron transport layer (ETL), an electron injection layer (EIL) and/or a charge generation layer (CGL). In one example embodiment, the emissive layer 220 can have a single emitting part (FIG. 3). Alternatively, the emissive layer 220 can have multiple emitting parts to form a tandem structure (FIG. 4). For example, the emissive layer 220 can be applied to an OLED with a single emitting part located in each of the red pixel region, the green pixel region and the blue pixel region. Alternatively, the emissive layer 220 can be applied to a tandem-type OLED where at least two emitting parts are stacked.

The emissive layer 220 can include the organic compound having the structure of Chemical Formulae 1 to 8. The luminous efficiency and the luminous lifespan of the OLED D and the organic light emitting display device 100 can be improved by including the organic compound having the structure of Chemical Formulae 1 to 8.

The second electrode 230 is disposed on the substrate 110 above which the emissive layer 220 is disposed. The second electrode 230 can be disposed on a whole display area. The second electrode 230 can include a conductive material with a relatively low work function value compared to the first electrode 210. The second electrode 230 can be a cathode providing electrons. For example, the second electrode 230 can include at least one of, but is not limited to, aluminum (Al), magnesium (Mg), calcium (Ca), silver (Ag), alloy thereof and/or combinations thereof such as aluminum-magnesium alloy (Al—Mg). When the organic light emitting display device 100 is a top-emission type, the second electrode 230 is thin so as to have light-transmissive (semi-transmissive) property.

In addition, an encapsulation film 180 can be disposed on the second electrode 230 in order to prevent or reduce outer moisture from penetrating into the OLED D. The encapsulation film 180 can have, but is not limited to, a laminated structure of a first inorganic insulating film 182, an organic insulating film 184 and a second inorganic insulating film 186. The encapsulation film 180 can be omitted.

A polarizing plate can be attached onto the encapsulation film 180 to reduce reflection of external light. For example, the polarizing plate may be a circular polarizing plate. When the organic light emitting display device 100 is a bottom-emission type, the polarizing plate can be disposed under the substrate 110. Alternatively, when the organic light emitting display device 100 is a top-emission type, the polarizing plate can be disposed on the encapsulation film 180. In addition, a cover window can be attached to the encapsulation film 180 or the polarizing plate. In this case, the substrate 110 and the cover window may have a flexible property, thus the organic light emitting display device 100 may be a flexible display device.

The OLED D is described in more detail. FIG. 3 illustrates a schematic cross-sectional view of an organic light emitting diode having a single emitting part in accordance with an example embodiment of the present disclosure. As illustrated in FIG. 3, the organic light emitting diode (OLED) D1 in accordance with the present disclosure includes first and second electrodes 210 and 230 facing each other and an emissive layer 220 disposed between the first and second electrodes 210 and 230. The organic light emitting display device 100 includes a red pixel region, a green pixel region and a blue pixel region, and the OLED D1 can be disposed in the red pixel region, the green pixel region and the blue pixel region. As an example, the OLED D1 can be disposed in the blue pixel region.

In an example embodiment, the emissive layer 220 includes an emitting material layer (EML) 340 disposed between the first and second electrodes 210 and 230. Also, the emissive layer 220 can include at least one of a hole transport layer (HTL) 320 disposed between the first electrode 210 and the EML 340 and an electron transport layer (ETL) 360 disposed between the second electrode 230 and the EML 340. In addition, the emissive layer 220 can further include at least one of a hole injection layer (HIL) 310 disposed between the first electrode 210 and the HTL 320 and an electron injection layer (EIL) 370 disposed between the second electrode 230 and the ETL 360. Alternatively, the emissive layer 220 can further comprise a first exciton blocking layer, i.e. an electron blocking layer (EBL) 330 disposed between the HTL 320 and the EML 340 and/or a second exciton blocking layer, i.e. a hole blocking layer (HBL) 350 disposed between the EML 340 and the ETL 360.

The first electrode 210 can be an anode that provides holes into the EML 340. The first electrode 210 can include a conductive material having a relatively high work function value, for example, a transparent conductive oxide (TCO). In an example embodiment, the first electrode 210 can include, but is not limited to, ITO, IZO, ITZO, SnO, ZnO, ICO, AZO, and/or combinations thereof.

The second electrode 230 can be a cathode that provides electrons into the EML 340. The second electrode 230 can include a conductive material having a relatively low work function values, i.e., a highly reflective material such as Al, Mg, Ca, Ag, and/or alloy thereof and/or combinations thereof such as Al—Mg.

The HIL 310 is disposed between the first electrode 210 and the HTL 320 and can improve an interface property between the inorganic first electrode 210 and the organic HTL 320. In one example embodiment, the HIL 310 can include, but is not limited to, 4,4′,4″-Tris(3-methylphenylamino)triphenylamine (MTDATA), 4,4′,4″-Tris(N,N-diphenyl-amino)triphenylamine (NATA), 4,4′,4″-Tris(N-(naphthalene-1-yl)-N-phenyl-amino)triphenylamine (1T-NATA), 4,4′,4″-Tris(N-(naphthalene-2-yl)-N-phenyl-amino)triphenylamine (2T-NATA), Copper phthalocyanine (CuPc), Tris(4-carbazoyl-9-yl-phenyl)amine (TCTA), N,N′-Diphenyl-N,N′-bis(1-naphthyl)-1,1′-biphenyl-4,4″-diamine (NPB; NPD), N,N′-Bis{4-[bis(3-methylphenyl)amino]phenyl}-N,N′-diphenyl-4,4′-biphenyldiamine (DNTPD), 1,4,5,8,9,11-Hexaazatriphenylenehexacarbonitrile (Dipyrazino[2,3-f:2′3′-h]quinoxaline-2,3,6,7,10,11-hexacarbonitrile; HAT-CN), 1,3,5-tris[4-(diphenylamino)phenyl]benzene (TDAPB), poly(3,4-ethylenedioxythiphene)polystyrene sulfonate (PEDOT/PSS), N-(biphenyl-4-yl)-9,9-dimethyl-N-(4-(9-phenyl-9H-carbazol-3-yl)phenyl)-9H-fluoren-2-amine, N,N′-diphenyl-N,N′-di[4-(N,N′-diphenyl-amino)phenyl]benzidine (NPNPB) and/or combinations thereof. The HIL 310 can be omitted in compliance of the OLED D1 property.

The HTL 320 is disposed adjacently to the EML 340 between the first electrode 210 and the EML 340. In one example embodiment, the HTL 320 can include, but is not limited to, N,N′-Diphenyl-N,N′-bis(3-methylphenyl)-1,1′-biphenyl-4,4′-diamine (TPD), NPB(NPD), DNTPD, 4,4′-bis(N-carbazolyl)-1,1′-biphenyl (CBP), Poly[N,N′-bis(4-butylpnehyl)-N,N′-bis(phenyl)-benzidine] (Poly-TPD), Poly[(9,9-dioctylfluorenyl-2,7-diyl)-co-(4,4′-(N-(4-sec-butylphenyl)diphenylamine))] (TFB), Di-[4-(N,N-di-p-tolyl-amino)-phenyl]cyclohexane (TAPC), 3,5-Di(9H-carbazol-9-yl)-N,N-diphenylaniline (DCDPA), N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine), N-([1,1′-Biphenyl]-4-yl)-9,9-dimethyl-N-(4-(9-phenl-9H-carbazol-3-yl)phenyl)-9H-fluoren-2-amine and/or combinations thereof.

The EML 340 can include a first host 342, and optionally, a second host 344 and/or an emitter (dopant) 346 where substantial light emission is occurred. The EML 340 can emit red, green and/or blue color light.

The first host 342 can be an N-type host (electron-type host) with relatively beneficial electron affinity compared to the second host 344. The first host 342 includes the organic compound having the structure of Chemical Formulae 1 to 8.

The second host 344 can be a P-type host (hole-type host) with relatively beneficial hole affinity compared to the first host 342. As an example, the second host 344 can include, but is not limited to, a biscarbazole-based organic compound, an aryl amine- or a hetero aryl amine-based organic compound with at least one fused aromatic and/or fused hetero aromatic moiety, and/or an aryl amine- or a hetero aryl amine-based organic compound with a spirofluorene moiety.

In one example embodiment, the second host 344 can include a carbazole-based organic compound where unsubstituted or substituted carbazole moieties located at both sides of a molecule are linked through plural unsubstituted or substituted phenylene rings. The second host 344 with such a molecular conformation can have the following structure of Chemical Formula 9:

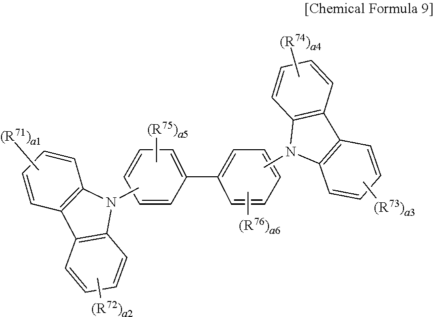

wherein, in the Chemical Formula 9,
each of R⁷¹, R⁷², R⁷³, R⁷⁴, R⁷⁵ and R⁷⁶ is independently an unsubstituted or substituted C₁-C₂₀ alkyl group, an unsubstituted or substituted C₆-C₃₀ aryl group or an unsubstituted or substituted C₃-C₃₀ hetero aryl group, where each R⁷¹ is identical to or different from each other when a1 is 2, 3 or 4, each R⁷² is identical to or different from each other when a2 is 2, 3 or 4, each R⁷³ is identical to or different from each other when a3 is 2, 3 or 4, each R⁷⁴ is identical to or different from each other when a4 is 2, 3 or 4, each R⁷⁵ is identical to or different from each other when a5 is 2, 3 or 4, and each R⁷⁶ is identical to or different from each other when a6 is 2, 3 or 4; and
each of a1, a2, a3, a4, a5 and a6 is independently 0, 1, 2, 3 or 4.

For example, each of R⁷¹, R⁷², R⁷³, R⁷⁴, R⁷⁵ and R⁷⁶ in Chemical Formula 9 can be independently an unsubstituted or substituted C₁-C₁₀ alkyl group (e.g., methyl or tert-butyl) or an unsubstituted or substituted C₆-C₃₀ aryl group (e.g., phenyl unsubstituted or substituted with a C₁-C₁₀ alkyl group such as methyl and/or tert-butyl). In addition, each of a1, a2, a3 and a4 in Chemical Formula 9 can be independently 0 or 1 and each of a5 and a6 in Chemical Formula 9 can be independently 0.

More particularly, the second host 344 having the structure of Chemical Formula 9 can be, but is not limited to, at least one of the following organic compounds of Chemical Formula 10:

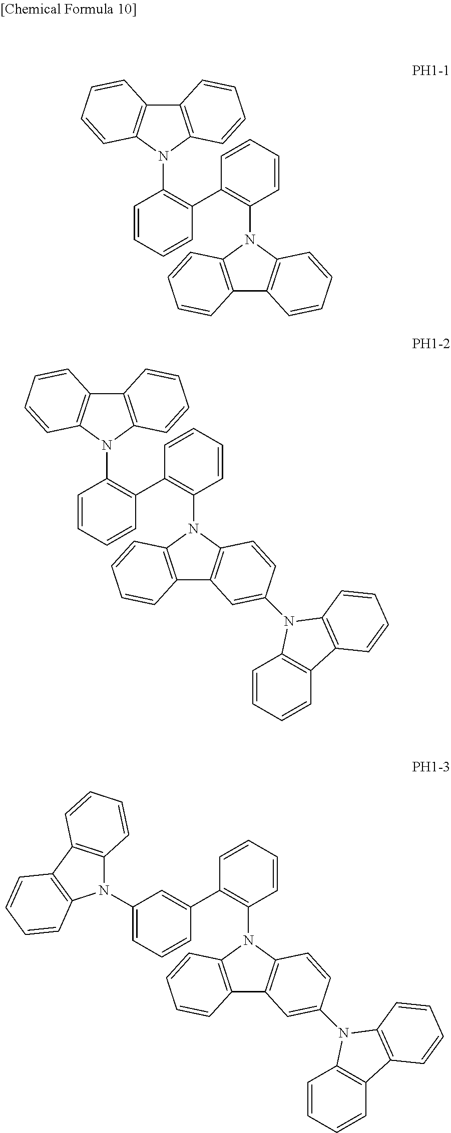

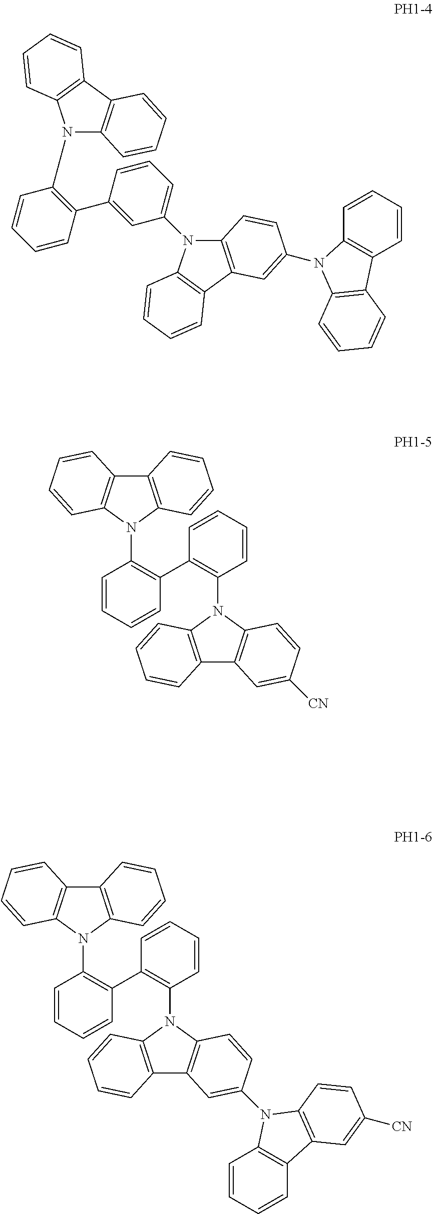 [SMILES: c1cc(-c2ccccc2-n2c3ccccc3c3ccccc32)cc(-n2c3ccccc3c3cc(-n4c5ccccc5c5ccccc54)ccc32)c1 ; N#Cc1ccc2c(c1)c1ccccc1n2-c1ccccc1-c1ccccc1-n1c2ccccc2c2ccccc21 ; N#Cc1ccc2c(c1)c1ccccc1n2-c1ccc2c(c1)c1ccccc1n2-c1ccccc1-c1ccccc1-n1c2ccccc2c2ccccc21]

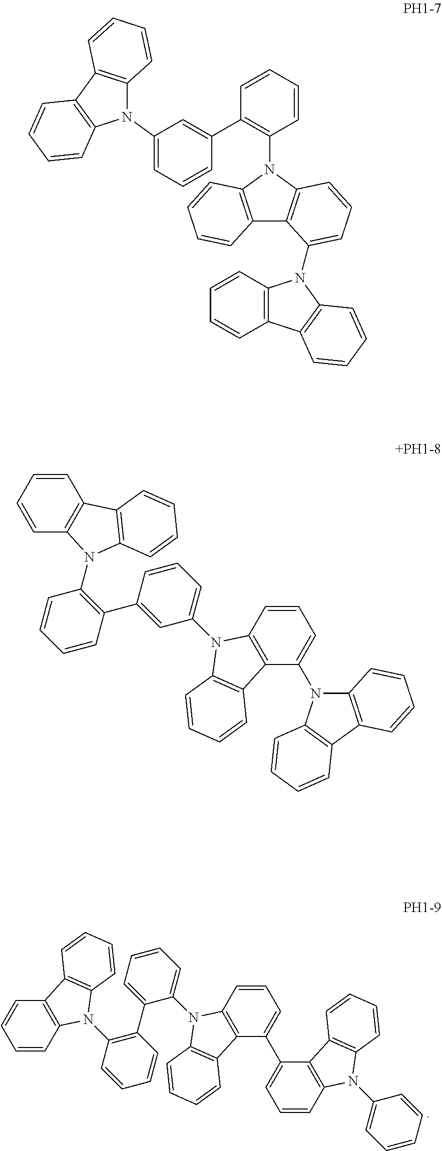 [SMILES: c1cc(-c2ccccc2-n2c3ccccc3c3c(-n4c5ccccc5c5ccccc54)cccc32)cc(-n2c3ccccc3c3ccccc32)c1 ; c1cc(-c2ccccc2-n2c3ccccc3c3ccccc32)cc(-n2c3ccccc3c3c(-n4c5ccccc5c5ccccc54)cccc32)c1 ; c1ccc(-n2c3ccccc3c3c(-c4cccc5c4c4ccccc4n5-c4ccccc4-c4ccccc4-n4c5ccccc5c5ccccc54)cccc32)cc1]

In another example embodiment, the second host 344 can include a carbazole-based organic compound where two carbazole moieties are linked directly to each other and at least one carbazole moiety is substituted. The second host 344 with such a molecular conformation can have the following structure of Chemical Formula 11:

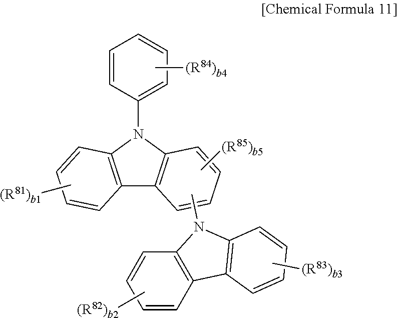

wherein, in the Chemical Formula 11,
each of R⁸¹, R⁸², R⁸³, R⁸⁴ and R⁸⁵ is independently an unsubstituted or substituted C₁-C₂₀ alkyl group, an unsubstituted or substituted C₆-C₃₀ aryl group or an unsubstituted or substituted C₃-C₃₀ hetero aryl group, where each R⁸¹ is identical to or different from each other when b1 is 2, 3 or 4, each R⁸² is identical to or different from each other when b2 is 2, 3 or 4, each R⁸³ is identical to or different from each other when b3 is 2, 3 or 4, where each R⁸⁴ is identical to or different from each other when b4 is 2, 3, 4 or 5, and each R⁸⁵ is identical to or different from each other when a5 is 2 or 3;
each of b1, b2 and b3 is independently 0, 1, 2, 3 or 4;
b4 is 0, 1, 2, 3, 4 or 5; and
b5 is 0, 1, 2 or 3.

For example, each of R⁸¹, R⁸², R⁸³, R⁸⁴ and R¹⁵ in Chemical Formula 11 can be independently an unsubstituted or substituted C₁-C₁₀ alkyl group (e.g., methyl or tert-butyl), an unsubstituted or substituted C₆-C₃₀ aryl group (e.g., phenyl unsubstituted or substituted with a C₁-C₁₀ alkyl group such as methyl and/or tert-butyl), an unsubstituted or substituted C₃-C₃₀ hetero aryl group (e.g., carbazolyl unsubstituted or substituted with a C₁-C₁₀ alkyl group such as methyl and/or tert-butyl), a tri-aryl methyl group (e.g., tri-phenyl methyl), a tri-aryl silyl group (e.g., tri-phenyl silyl) or a tri-aryl germanyl group (e.g., tri-phenyl germanyl). In addition, each of b1, b2 and b5 can be independently 0 and each of b3 and b4 can be independently 0 or 1 in Chemical Formula 11.

More particularly, the second host 344 having the structure of Chemical Formula 11 can be, but is not limited to, at least one of the following organic compounds of Chemical Formula 12:

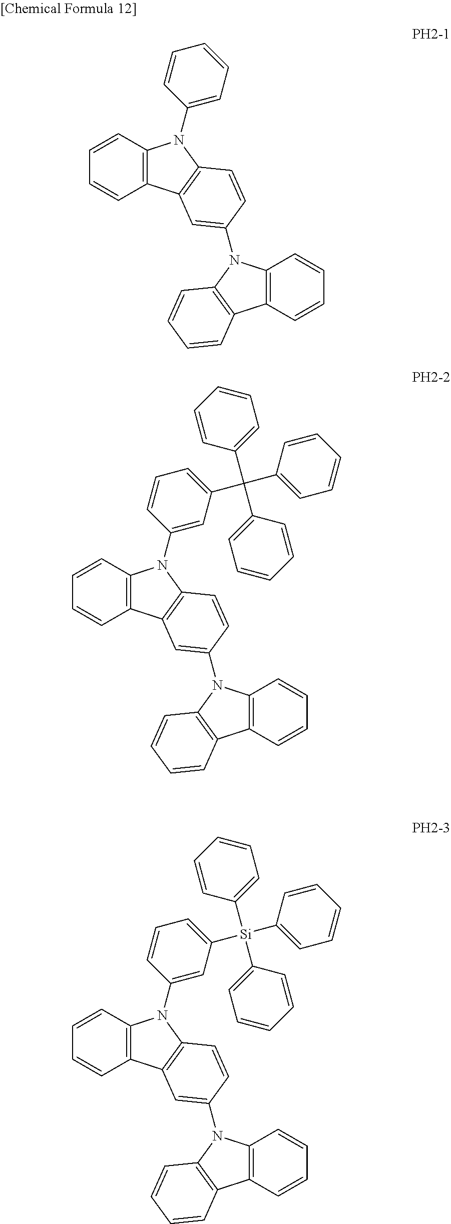

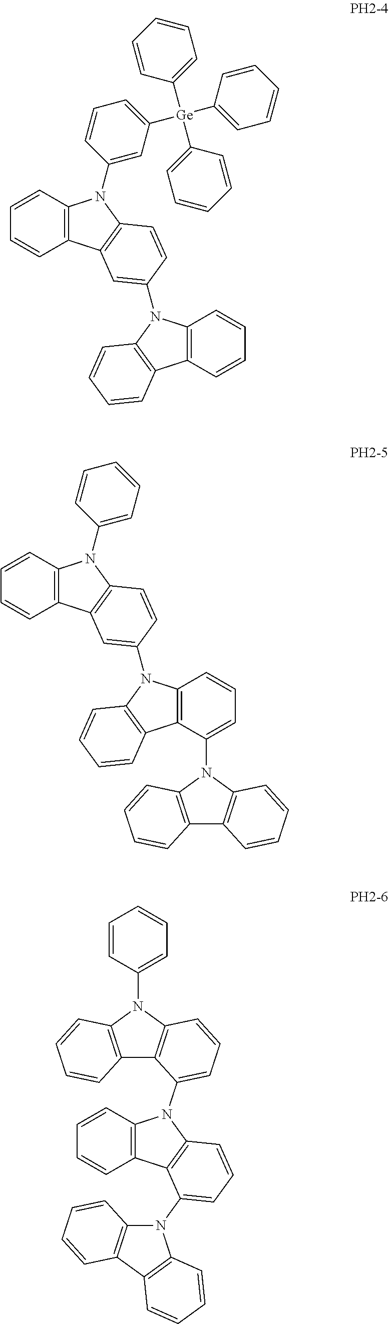

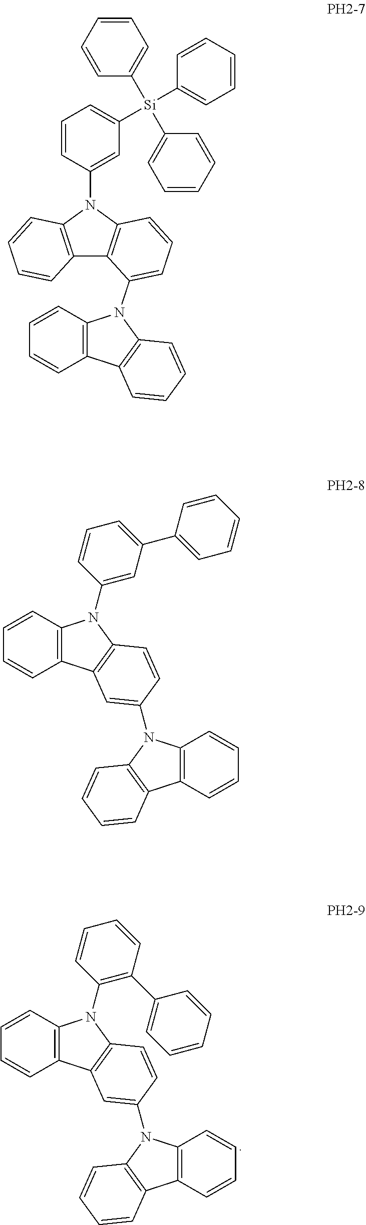 [SMILES: c1ccc([Si](c2ccccc2)(c2ccccc2)c2cccc(-n3c4ccccc4c4c(-n5c6ccccc6c6ccccc65)cccc43)c2)cc1 ; c1ccc(-c2cccc(-n3c4ccccc4c4cc(-n5c6ccccc6c6ccccc65)ccc43)c2)cc1 ; c1ccc(-c2ccccc2-n2c3ccccc3c3cc(-n4c5ccccc5c5ccccc54)ccc32)cc1]

In an alternative embodiment, the second host 344 can include, but is not limited to, 9-(3-(9H-carbazol-9-yl)phenyl)-9H-carbazole-3-carbonitrile (mCP-CN), CBP, 3,3′-bis(N-carbazolyl)-1,1′-biphenyl (mCBP), 1,3-Bis(carbazol-9-yl)benzene (mCP), Bis[2-(diphenylphosphino)phenyl]ether oxide (DPEPO), 2,8-bis(diphenylphosphoryl)dibenzothiophene (PPT), 1,3,5-Tri[(3-pyridyl)-phen-3-yl]benzene (TmPyPB), 2,6-Di(9H-carbazol-9-yl)pyridine (PYD-2Cz), 2,8-di(9H-carbazol-9-yl)dibenzothiophene (DCzDBT), 3′,5′-Di(carbazol-9-yl)-[1,1′-bipheyl]-3,5-dicarbonitrile (DCzTPA), 4′-(9H-carbazol-9-yl)biphenyl-3,5-dicarbonitrile (pCzB-2CN), 3′-(9H-carbazol-9-yl)biphenyl-3,5-dicarbonitrile (mCzB-2CN), Diphenyl-4-triphenylsilyl-phenylphosphine oxide (TSPO1), 9-(9-phenyl-9H-carbazol-6-yl)-9H-carbazole (CCP), 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicabazole, 9,9′-Diphenyl-9H,9′H-3,3′-bicarbazole (BCzPh), 9,9′-Di(4-biphenyl)-9H,9′H-3,3′-bicabazole (BCZ), 1,3,5-Tris(carbazole-9-yl)benzene (TCP), TCTA, 4,4′-Bis(carbazole-9-yl)-2,2′-dimethylbipheyl (CDBP), (2,7-Bis(carbazole-9-yl)-9,9-dimethylfluorene (DMFL-CBP), 2,2′,7,7′-Tetrakis(carbazole-9-yl)-9,9-spirofluorene (Spiro-CBP), 3,6-Bis(carbazole-9-yl)-9-(2-ethyl-hexyl)-9H-carbazole (TCzl) and/or combinations thereof.

The emitter 346 can emit one of a blue color, a green color, a yellow-green color and a red color. As an example, the emitter 346 can emit a blue color. In addition, the emitter 346 can include at least one of phosphorescent material, fluorescent material and delayed fluorescent material.

For example, the emitter 346 having delayed fluorescent property can include any delayed fluorescent material emitting blue color. In one example embodiment, the emitter 346 having the delayed fluorescent property can include a boron-based organic compound where boron and oxygen atoms constitute at least one hetero aromatic rings. The emitter 346 with such a molecular conformation can include an organic compound having the following structure of Chemical Formula 13:

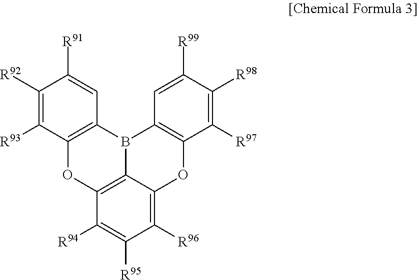

wherein, in the Chemical Formula 13,
each of R⁹¹, R⁹², R⁹³, R⁹⁴, R⁹⁵, R⁹⁶, R⁹⁷, R⁹⁸ and R⁹⁹ is independently hydrogen, halogen, an unsubstituted or substituted C₁-C₂₀ alkyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl amino group or an unsubstituted or substituted C₃-C₃₀ hetero aryl amino group, where one to four, for example, one to two among R⁹¹, R⁹², R⁹³, R⁹⁴, R⁹⁵, R⁹⁶, R⁹⁷, R⁹⁸ and R⁹⁹ is a moiety having the following structure of Chemical Formula 14,

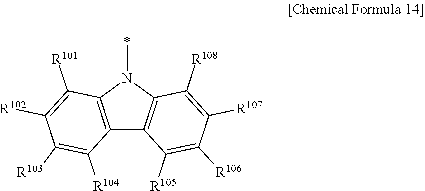

wherein, in the Chemical Formula 14,
each of R¹⁰¹, R¹⁰², R¹⁰³, R¹⁰⁴, R¹⁰⁵, R¹⁰⁶, R¹⁰⁷ and R¹⁰⁸ is independently hydrogen, an unsubstituted or substituted C₁-C₂₀ alkyl group, an unsubstituted or substituted C₁-C₂₀ alkyl silyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl amino group or an unsubstituted or substituted C₃-C₃₀ hetero aryl amino group, where at least two adjacent groups among R¹⁰¹, R¹⁰², R¹⁰³, R¹⁰⁴, R¹⁰⁵, R¹⁰⁶, R¹⁰⁷ and R¹⁰⁸ are linked to form the following hetero aromatic ring of Chemical Formula 15,

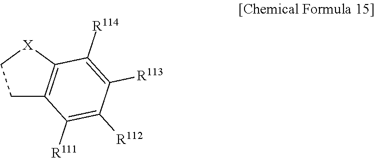

wherein, in the Chemical Formula 15,
X is NR¹¹⁵, O or S;
each of R¹¹¹, R¹¹², R¹¹³, R¹¹⁴ and R¹¹⁵ is independently hydrogen, halogen, an unsubstituted or substituted C₁-C₂₀ alkyl group, an unsubstituted or substituted C₁-C₂₀ alkyl silyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl silyl group, an unsubstituted or substituted C₃-C₃₀ hetero aryl silyl group, an unsubstituted or substituted C₆-C₃₀ aryl amino group or an unsubstituted or substituted C₃-C₃₀ hetero aryl group, and
a dotted line indicates a fused portion.

For example, each of the C₆-C₃₀ aryl group and the C₃-C₃₀ hetero aryl group of R⁹¹ to R⁹⁹ in Chemical Formula 13, R¹⁰¹ to R¹⁰⁸ in Chemical Formula 14 and R¹¹¹ to R¹¹⁵ in Chemical Formula 15 can be independently unsubstituted or substituted with at least one group of C₁-C₁₀ alkyl (e.g., C₁-C₅ alkyl such as tert-butyl), C₆-C₃₀ aryl (e.g., C₆-C₁₅ aryl group such as phenyl), C₃-C₃₀ hetero aryl (e.g., C₃-C₁₅ hetero aryl such as pyridyl) and C₆-C₂₀ aryl amino (e.g., diphenyl amino).

The fused ring including boron and oxygen atoms in Chemical Formula 13 acts as an electron acceptor group and the fused hetero aromatic ring having at least one nitrogen atom having the structure of Chemical Formula 14 acts as an electron donor group. The organic compound having the structure of Chemical Formulae 13 to 15 has delayed fluorescent property.

As an example, the emitter 346 with the delayed fluorescent property having the structure of Chemical Formulae 13 to 15 can be, but is not limited to, at least one of the following organic compounds of Chemical Formula 16:

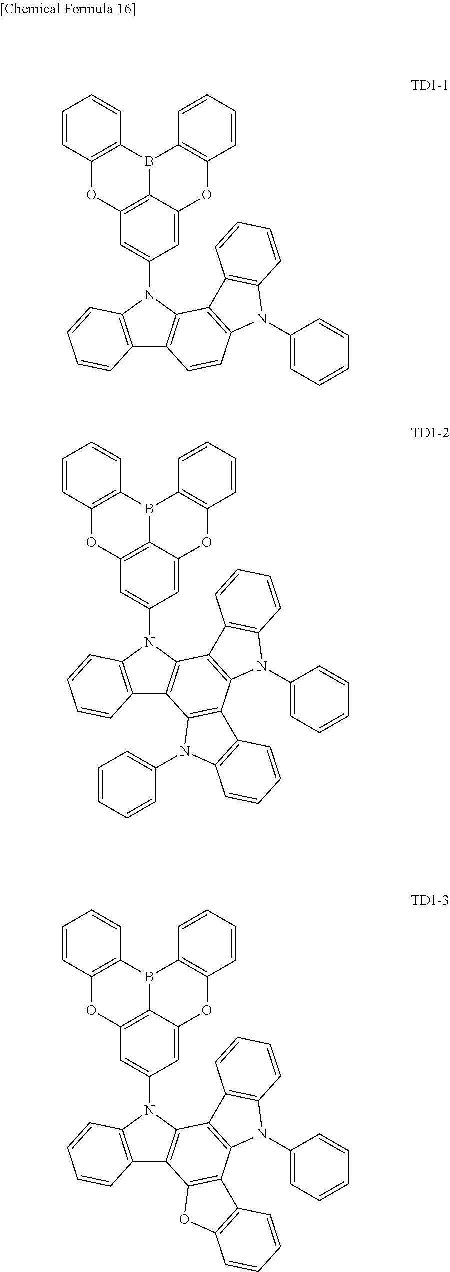 [SMILES: c1ccc(-n2c3ccccc3c3c2ccc2c4ccccc4n(-c4cc5c6c(c4)Oc4ccccc4B6c4ccccc4O5)c23)cc1 ; c1ccc(-n2c3ccccc3c3c2c2c4ccccc4n(-c4cc5c6c(c4)Oc4ccccc4B6c4ccccc4O5)c2c2c4ccccc4n(-c4ccccc4)c32)cc1 ; c1ccc(-n2c3ccccc3c3c4c(c5ccccc5n4-c4cc5c6c(c4)Oc4ccccc4B6c4ccccc4O5)c4oc5ccccc5c4c32)cc1]

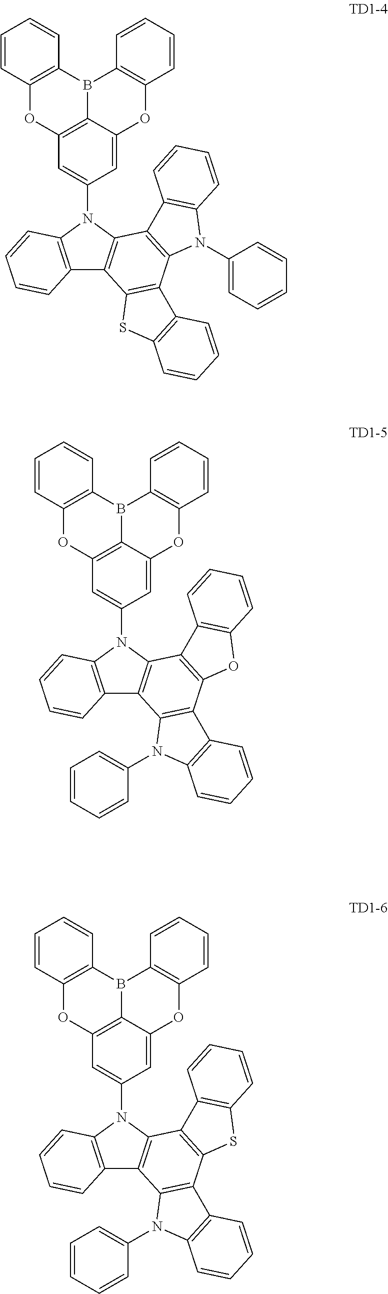 [SMILES: c1ccc(-n2c3ccccc3c3c4c(c5ccccc5n4-c4cc5c6c(c4)Oc4ccccc4B6c4ccccc4O5)c4sc5ccccc5c4c32)cc1 ; c1ccc(-n2c3ccccc3c3c4oc5ccccc5c4c4c(c5ccccc5n4-c4cc5c6c(c4)Oc4ccccc4B6c4ccccc4O5)c32)cc1 ; c1ccc(-n2c3ccccc3c3c4sc5ccccc5c4c4c(c5ccccc5n4-c4cc5c6c(c4)Oc4ccccc4B6c4ccccc4O5)c32)cc1]

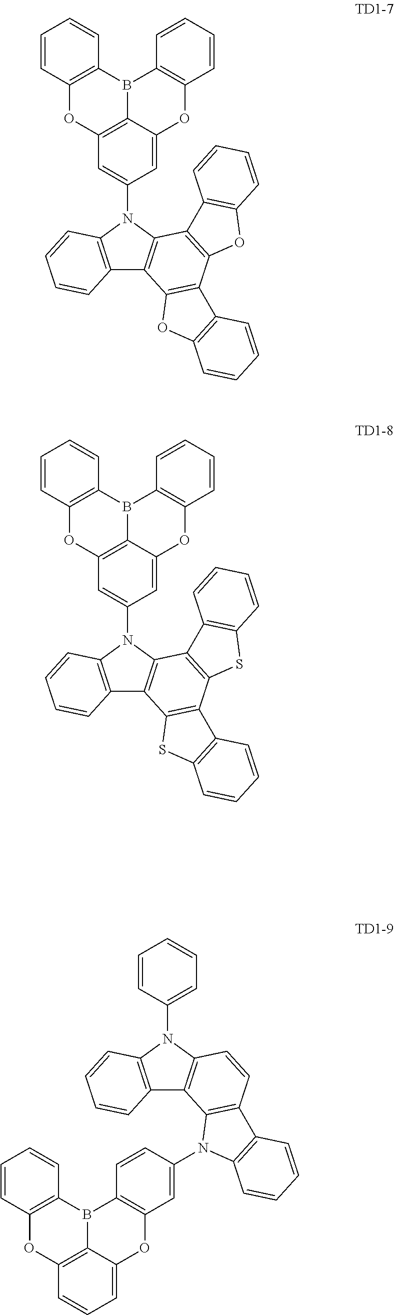 [SMILES: c1ccc2c(c1)Oc1cc(-n3c4ccccc4c4c5oc6ccccc6c5c5oc6ccccc6c5c43)cc3c1B2c1ccccc1O3 ; c1ccc2c(c1)Oc1cc(-n3c4ccccc4c4c5sc6ccccc6c5c5sc6ccccc6c5c43)cc3c1B2c1ccccc1O3 ; c1ccc(-n2c3ccccc3c3c2ccc2c4ccccc4n(-c4ccc5c(c4)Oc4cccc6c4B5c4ccccc4O6)c23)cc1]

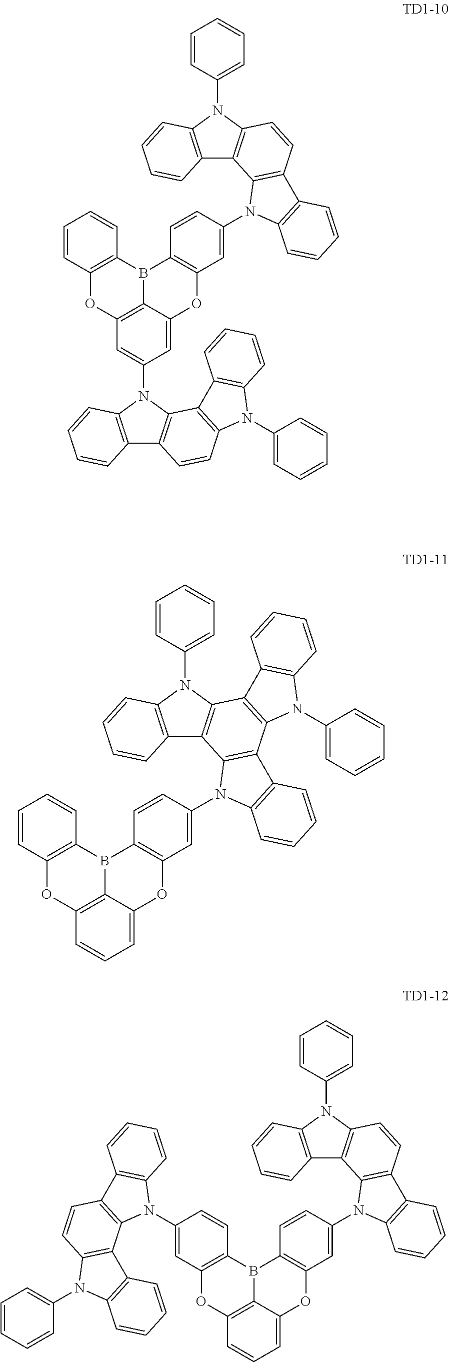 [SMILES: c1ccc(-n2c3ccccc3c3c2ccc2c4ccccc4n(-c4ccc5c(c4)Oc4cc(-n6c7ccccc7c7ccc8c(c9ccccc9n8-c8ccccc8)c76)cc6c4B5c4ccccc4O6)c23)cc1 ; c1ccc(-n2c3ccccc3c3c2c2c4ccccc4n(-c4ccc5c(c4)Oc4cccc6c4B5c4ccccc4O6)c2c2c4ccccc4n(-c4ccccc4)c32)cc1 ; c1ccc(-n2c3ccccc3c3c2ccc2c4ccccc4n(-c4ccc5c(c4)Oc4cccc6c4B5c4ccc(-n5c7ccccc7c7ccc8c(c9ccccc9n8-c8ccccc8)c75)cc4O6)c23)cc1]

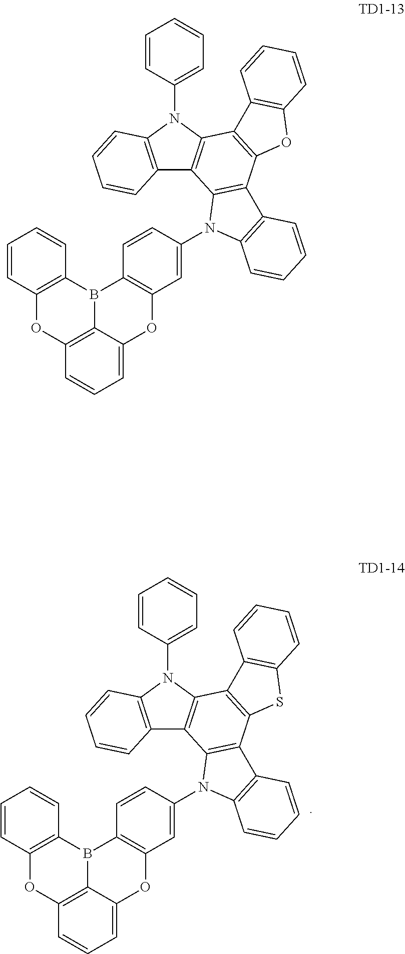 [SMILES: c1ccc(-n2c3ccccc3c3c4c(c5ccccc5n4-c4ccc5c(c4)Oc4cccc6c4B5c4ccccc4O6)c4oc5ccccc5c4c32)cc1 ; c1ccc(-n2c3ccccc3c3c4c(c5ccccc5n4-c4ccc5c(c4)Oc4cccc6c4B5c4ccccc4O6)c4sc5ccccc5c4c32)cc1]

In another example embodiment, the emitter 346 having the delayed fluorescent property can include a triazine moiety of an electron acceptor moiety and a carbazolyl moiety of an electron donor moiety. The emitter 346 with such a molecular conformation can include an organic compound having the following structure of Chemical Formula 17:

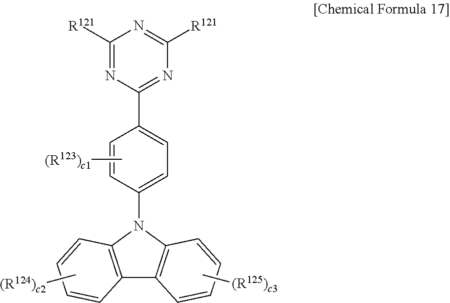

wherein, in the Chemical Formula 17,
each of R¹²¹, R¹²², R¹²³, R¹²⁴ and R¹²⁵ is independently an unsubstituted or substituted C₆-C₃₀ aryl group or an unsubstituted or substituted C₃-C₃₀ hetero aryl group, where each R¹²³ is identical to or different from each other when c1 is 2, 3 or 4, each R¹²⁴ is identical to or different from each other when c2 is 2, 3 or 4 and each R¹²⁵ is identical to or different from each other when c3 is 2, 3 or 4, or optionally,
two adjacent R¹²⁴ when c2 is 2, 3 or 4 and/or two adjacent R¹²⁵ when c3 is 2, 3 or 4 can be further linked to form an unsubstituted or substituted C₆-C₂₀ aromatic ring or an unsubstituted or substituted C₃-C₂₀ aromatic ring; and
each of c1, c2 and c3 is independently 0, 1, 2, 3 or 4.

For example, each of R¹²¹ to R¹²⁵ can be independently phenyl, dibenzo-furanyl or dibenzo-thiophenyl, two adjacent R¹²⁴ and/or two adjacent R¹²⁵ can be further linked to form an indene ring, an indole ring, a benzofuran ring or a benzothiophene ring, c1 can be 1 or 2 and/or each of c2 and c3 can be independently 0 or 1 in Chemical Formula 17.

As an example, the emitter 346 with delayed fluorescent property having the structure of Chemical Formula 17 can be, but is not limited to, at least one of the organic compounds of Chemical Formula 18:

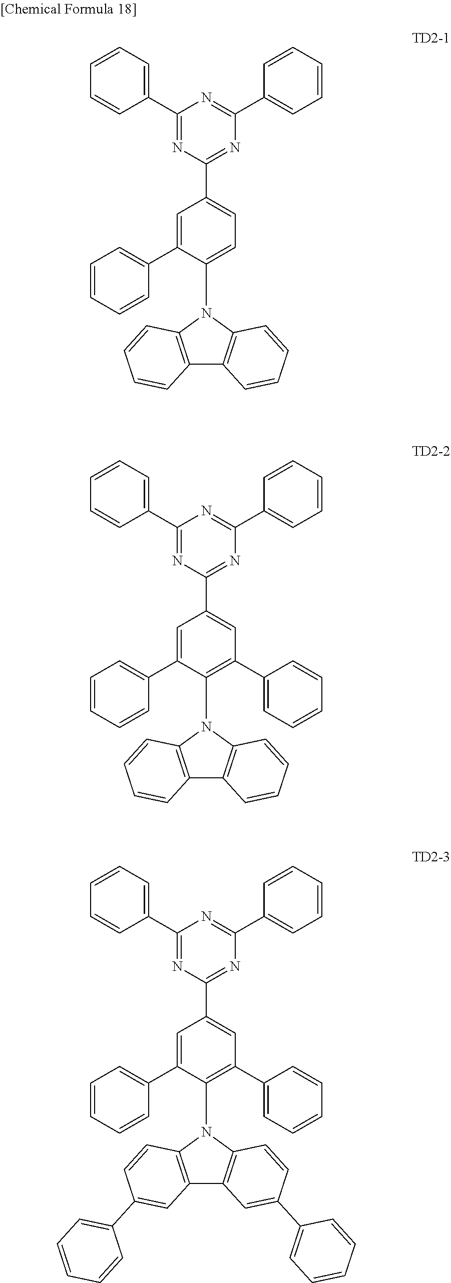 [SMILES: c1ccc(-c2nc(-c3ccccc3)nc(-c3ccc(-n4c5ccccc5c5ccccc54)c(-c4ccccc4)c3)n2)cc1 ; c1ccc(-c2nc(-c3ccccc3)nc(-c3cc(-c4ccccc4)c(-n4c5ccccc5c5ccccc54)c(-c4ccccc4)c3)n2)cc1 ; c1ccc(-c2ccc3c(c2)c2cc(-c4ccccc4)ccc2n3-c2c(-c3ccccc3)cc(-c3nc(-c4ccccc4)nc(-c4ccccc4)n3)cc2-c2ccccc2)cc1]

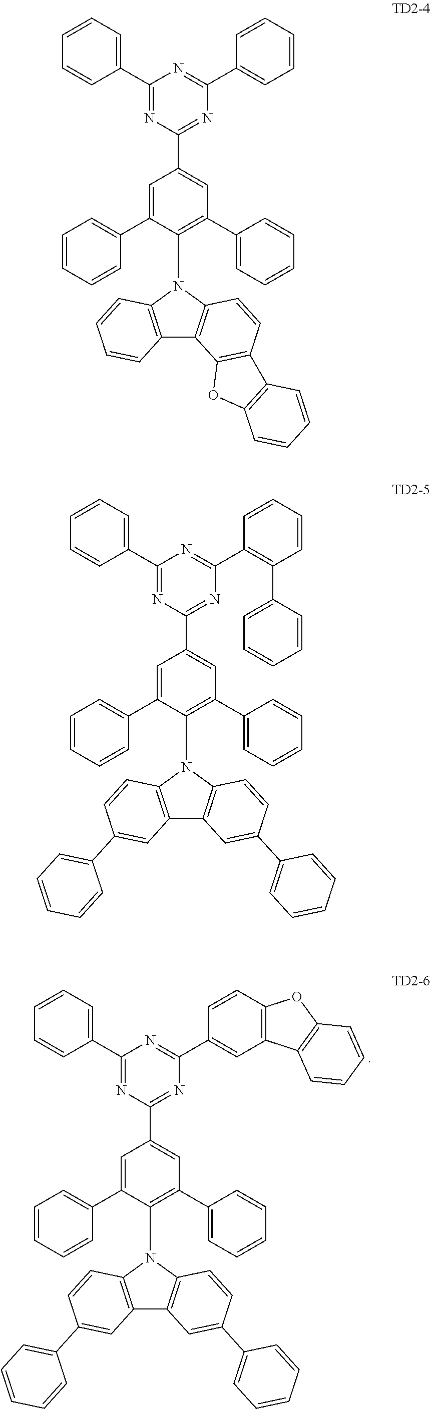 [SMILES: c1ccc(-c2nc(-c3ccccc3)nc(-c3cc(-c4ccccc4)c(-n4c5ccccc5c5c6oc7ccccc7c6ccc54)c(-c4ccccc4)c3)n2)cc1 ; c1ccc(-c2ccc3c(c2)c2cc(-c4ccccc4)ccc2n3-c2c(-c3ccccc3)cc(-c3nc(-c4ccccc4)nc(-c4ccccc4-c4ccccc4)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5oc6ccccc6c5c4)n3)cc2-c2ccccc2)cc1]

In still another example embodiment, the emitter 346 having the delayed fluorescent property can include a cyano group moiety of an electron acceptor moiety and a carbazolyl moiety of an electron donor moiety. The emitter 346 with such a molecular conformation can have an organic compound having the following structure of Chemical Formula 19:

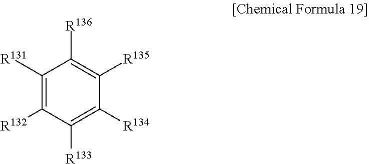

wherein, in the Chemical Formula 19,
each of R¹³¹, R¹³², R¹³³, R¹³⁴, R¹³⁵ and R¹³⁶ is independently a cyano group, an unsubstituted or substituted C₆-C₃₀ aryl group or an unsubstituted or substituted C₃-C₃₀ hetero aryl group, where at least one of R¹³¹, R¹³², R¹³³, R¹³⁴, R¹³⁵ and R¹³⁶ is a cyano group or phenyl substituted with a cyano group, and at least two of R¹³¹, R¹³², R¹³³, R¹³⁴, R¹³⁵ and R¹³⁶ are unsubstituted or substituted carbazolyl.

For example, R¹³¹, R¹³², R¹³³, R¹³⁴, R¹³⁵ and R¹³⁶ can be independently a cyano group, phenyl unsubstituted or substituted with cyano and/or phenyl, or carbazolyl unsubstituted or substituted with phenyl, at least one of R¹³¹, R¹³², R¹³³, R¹³⁴, R¹³⁵ and R¹³⁶ can be a cyano group or cyano-substituted phenyl, and at least two (for example at least three or at least four) of R¹³¹, R¹³², R¹³³, R¹³⁴, R¹³⁵ and R¹³⁶ can be unsubstituted or phenyl-substituted carbazolyl in Chemical Formula 19.

As an example, the emitter 346 with the delayed fluorescent properties having the structure of Chemical Formula 19 can be, but is not limited to, at least one of the following organic compounds of Chemical Formula 20:

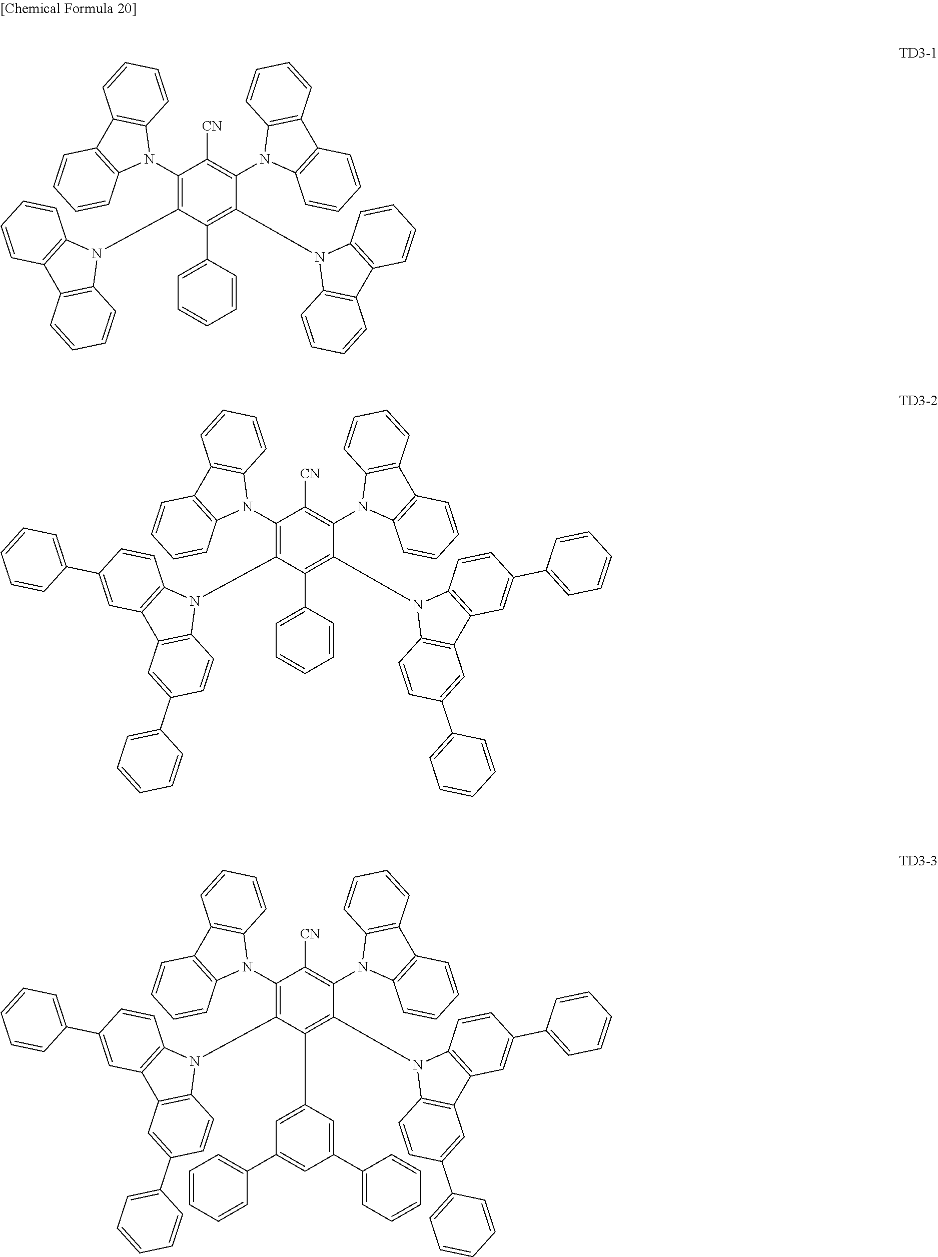 [SMILES: N#Cc1c(-n2c3ccccc3c3ccccc32)c(-n2c3ccccc3c3ccccc32)c(-c2ccccc2)c(-n2c3ccccc3c3ccccc32)c1-n1c2ccccc2c2ccccc21 ; N#Cc1c(-n2c3ccccc3c3ccccc32)c(-n2c3ccc(-c4ccccc4)cc3c3cc(-c4ccccc4)ccc32)c(-c2ccccc2)c(-n2c3ccc(-c4ccccc4)cc3c3cc(-c4ccccc4)ccc32)c1-n1c2ccccc2c2ccccc21 ; N#Cc1c(-n2c3ccccc3c3ccccc32)c(-n2c3ccc(-c4ccccc4)cc3c3cc(-c4ccccc4)ccc32)c(-c2cc(-c3ccccc3)cc(-c3ccccc3)c2)c(-n2c3ccc(-c4ccccc4)cc3c3cc(-c4ccccc4)ccc32)c1-n1c2ccccc2c2ccccc21]

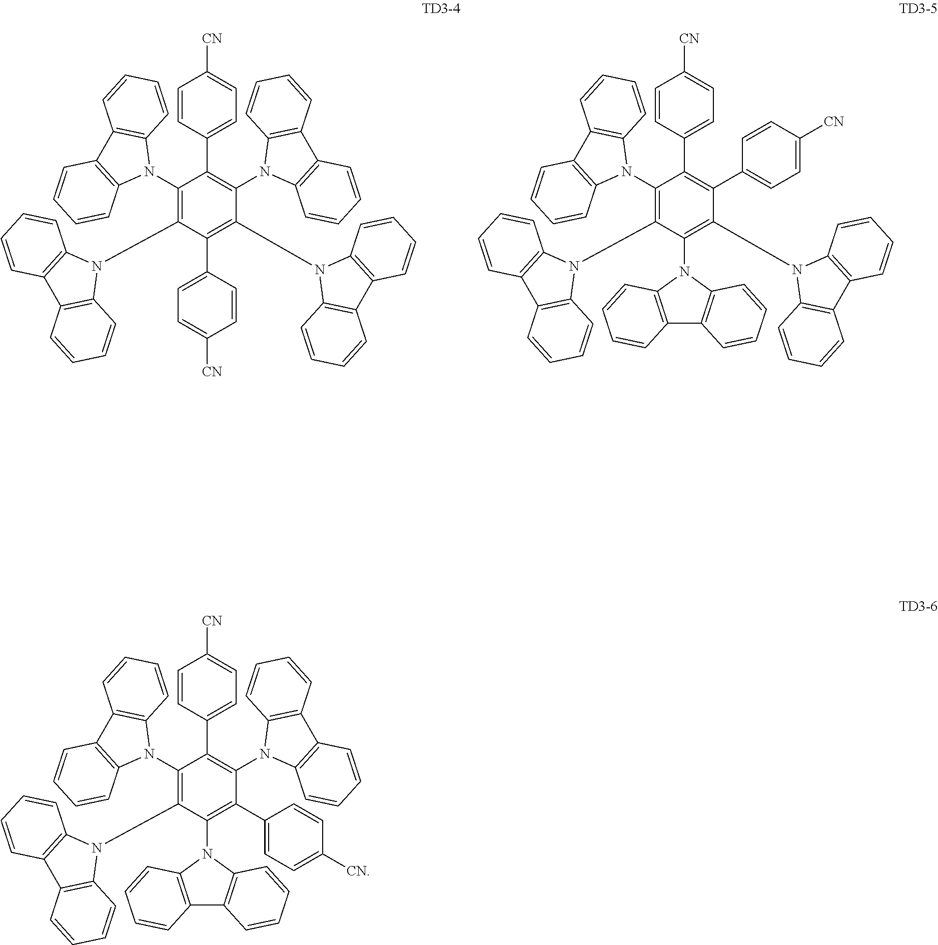 [SMILES: N#Cc1ccc(-c2c(-n3c4ccccc4c4ccccc43)c(-n3c4ccccc4c4ccccc43)c(-c3ccc(C#N)cc3)c(-n3c4ccccc4c4ccccc43)c2-n2c3ccccc3c3ccccc32)cc1 ; N#Cc1ccc(-c2c(-c3ccc(C#N)cc3)c(-n3c4ccccc4c4ccccc43)c(-n3c4ccccc4c4ccccc43)c(-n3c4ccccc4c4ccccc43)c2-n2c3ccccc3c3ccccc32)cc1 ; N#Cc1ccc(-c2c(-n3c4ccccc4c4ccccc43)c(-c3ccc(C#N)cc3)c(-n3c4ccccc4c4ccccc43)c(-n3c4ccccc4c4ccccc43)c2-n2c3ccccc3c3ccccc32)cc1]

The emitter 346 having phosphorescent properties can include any phosphorescent material emitting blue color light. As an example, the emitter 346 having phosphorescent property include an organometallic compound with platinum atom of a center coordination atom, and can include, but is not limited to, an organometallic compound having the structure of Chemical Formula 21:

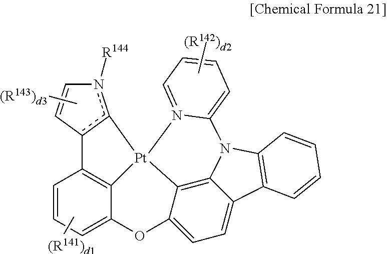

wherein, in the Chemical Formula 21,
each of R¹⁴¹, R¹⁴² and R¹⁴³ is independently an unsubstituted or substituted C₁-C₂₀ alkyl group, an unsubstituted or substituted C₆-C₃₀ aryl group or an unsubstituted or substituted C₃-C₃₀ hetero aryl group, where each R¹⁴¹ is identical to or different from each other when d1 is 2 or 3, each R¹⁴² is identical to or different from each other when d2 is 2, 3 or 4, and each R¹⁴³ is identical to or different from each other when d3 is 2, or
optionally, two adjacent R¹⁴³ are further linked to form an unsubstituted or substituted C₆-C₂₀ aromatic ring or an unsubstituted or substituted C₃-C₂₀ hetero aromatic ring;
R₁₄₄ is hydrogen, an unsubstituted or substituted C₁-C₂₀ alkyl group, an unsubstituted or substituted C₆-C₃₀ aryl group or an unsubstituted or substituted C₃-C₃₀ hetero aryl group;
d1 is 0, 1, 2 or 3;
d2 is 0, 1, 2, 3 or 4; and
d3 is 0, 1, 2.

In one example embodiment, each of R¹⁴¹, R¹⁴², R¹⁴³ and R¹⁴⁴ can be independently an unsubstituted or substituted C₁-C₁₀ alkyl group (e.g., methyl or tert-butyl), two adjacent R¹⁴³ can be further linked to form a benzene ring, d1 can be 0 and/or each of d2 and d3 can be independently 0, 1 or 2 in Chemical Formula 21.

As an example, the emitter 346 with phosphorescent property having the structure of Chemical Formula 21 can be, but is not limited to, at least one of the following organometallic compounds of Chemical Formula 22:

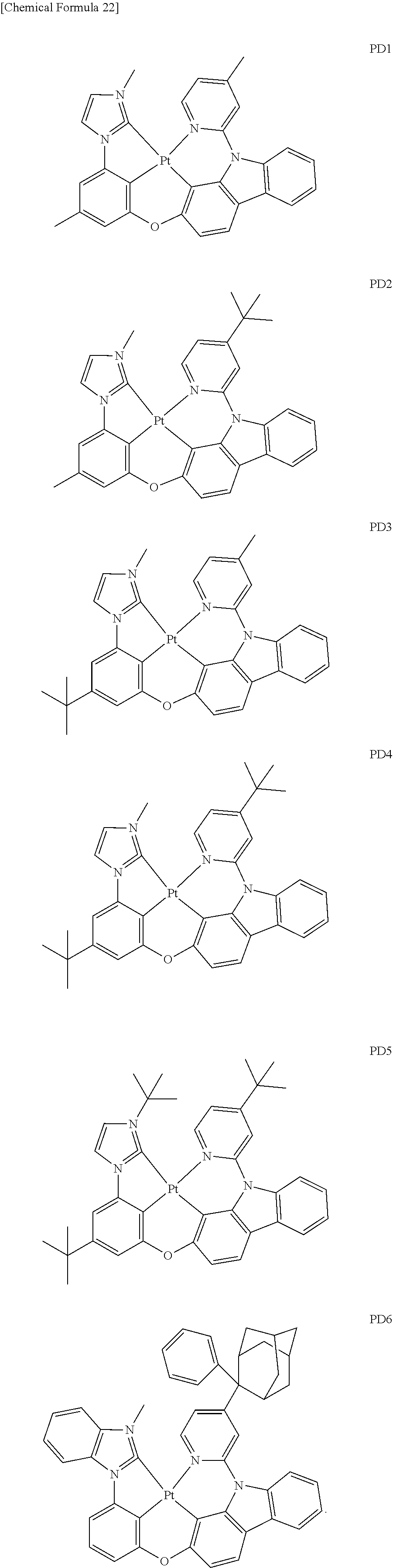

The emitter 346 having fluorescent property can includes any fluorescent material emitting blue color light. In one example embodiment, the emitter 346 having fluorescent property can include a boron-based organic compound where boron and nitrogen atoms constitute a ring system. The emitter 346 with such a molecular conformation can include an organic compound having the following structure of Chemical Formula 23:

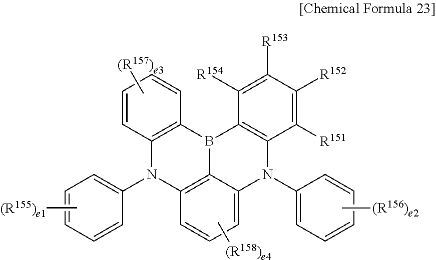

wherein, in the Chemical Formula 23,
each of R¹⁵¹, R¹⁵², R¹⁵³, R¹⁵⁴, R¹⁵⁵, R¹⁵⁶, R¹⁵⁷ and R¹⁵⁸ is independently hydrogen, halogen, an unsubstituted or substituted C₁-C₂₀ alkyl group, an unsubstituted or substituted C₁-C₂₀ alkyl silyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl amino group or an unsubstituted or substituted C₃-C₃₀ hetero aryl amino group, where each R¹⁵⁵ is identical to or different from each other when e1 is 2, 3, 4 or 5, each R¹⁵⁶ is identical to or different from each other when e2 is 2, 3, 4 or 5, each R¹⁵⁷ is identical to or different from each other when e3 is 2, 3 or 4, and each R¹⁵⁸ is identical to or different from each other when e4 is 2 or 3, or
optionally,
two adjacent groups among R¹⁵¹, R¹⁵², R¹⁵³ and R¹⁵⁴ can be further linked to form a fused ring including a boron atom and a nitrogen atom;
each of e1 and e2 is independently 0, 1, 2, 3, 4 or 5;
e3 is 0, 1, 2, 3 or 4; and
e4 is 0, 1, 2 or 3.

The boron-based fluorescent compound having the structure of Chemical Formula 23 has beneficial luminous efficiency and a wide plate-like structure. When the EML 340 includes the organic compound having the structure of Chemical Formula 1 alone or together with other delayed fluorescent compound, the luminous efficiency of the OLED D1 can be maximized or increased. As an example, the emitter 346 with fluorescent property having the structure of Chemical Formula 23 can be, but is not limited to, at least one of the following organic compounds of Chemical Formula 24:

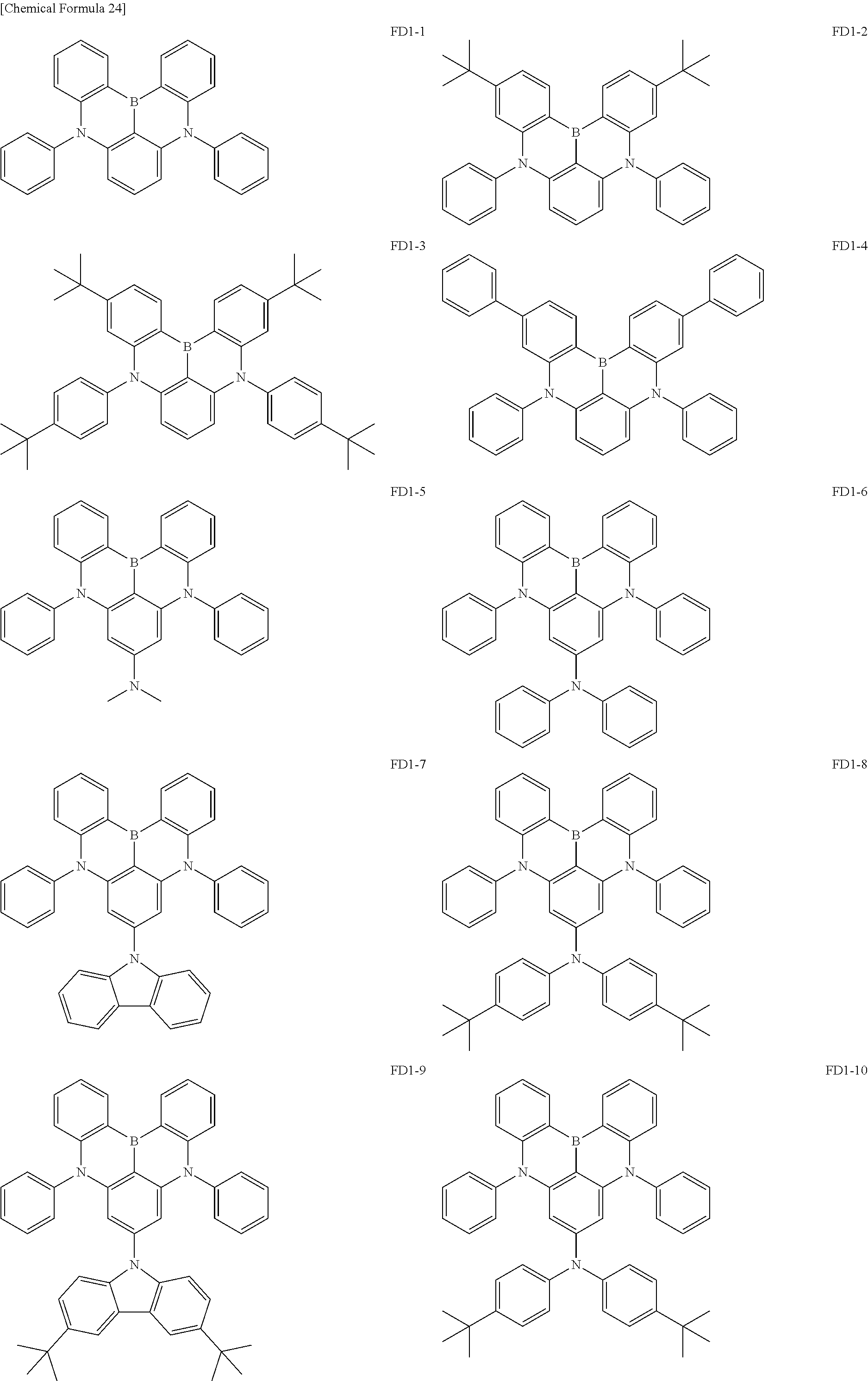

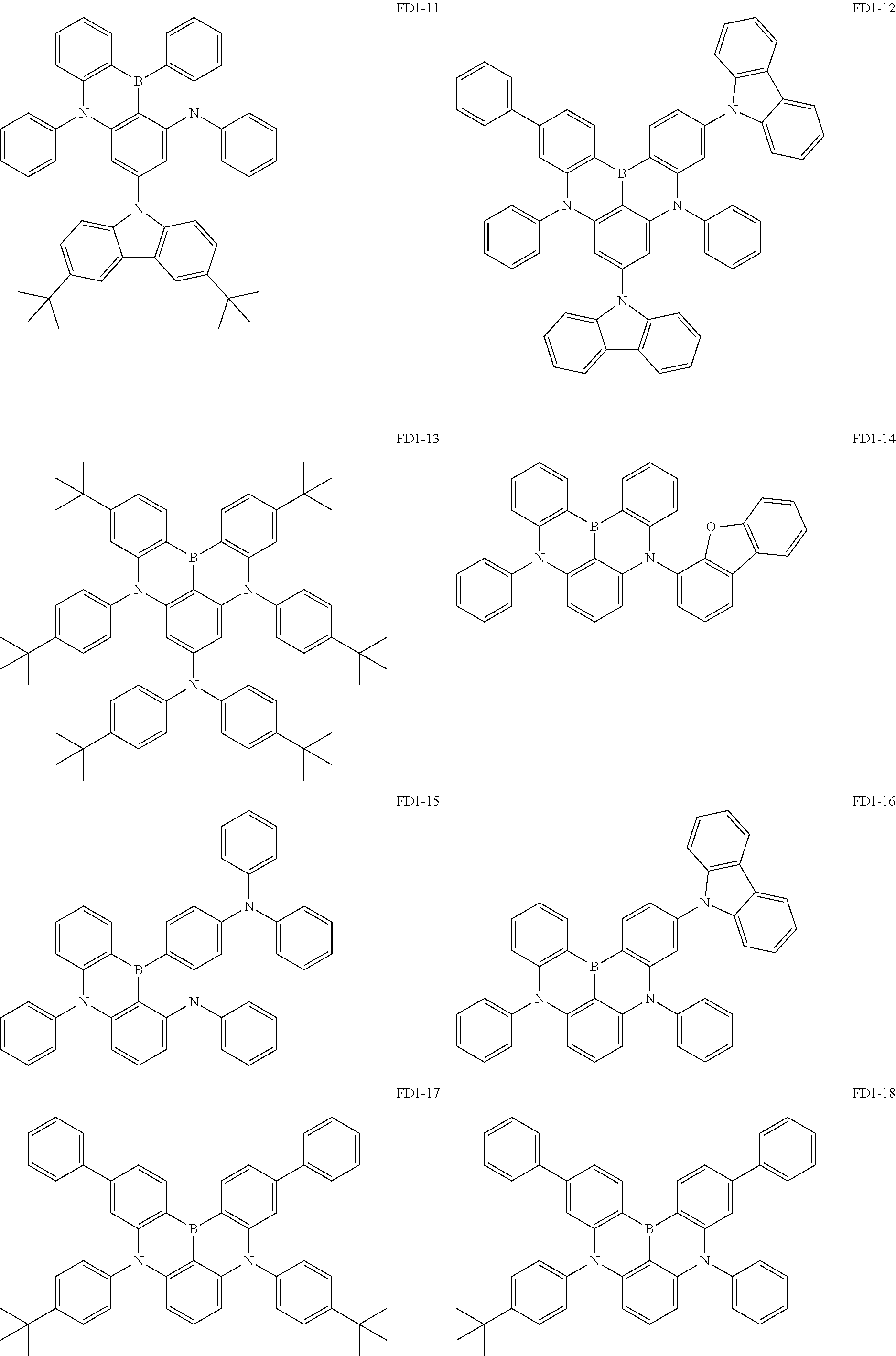

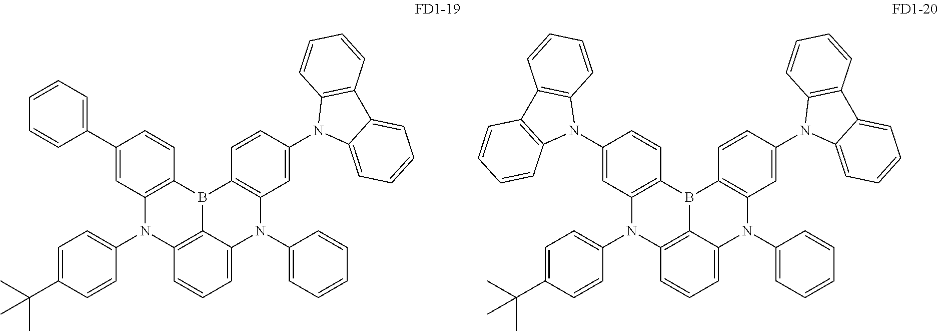 [SMILES: CC(C)(C)c1ccc(N2c3cc(-c4ccccc4)ccc3B3c4ccc(-n5c6ccccc6c6ccccc65)cc4N(c4ccccc4)c4cccc2c43)cc1 ; CC(C)(C)c1ccc(N2c3cc(-n4c5ccccc5c5ccccc54)ccc3B3c4ccc(-n5c6ccccc6c6ccccc65)cc4N(c4ccccc4)c4cccc2c43)cc1]

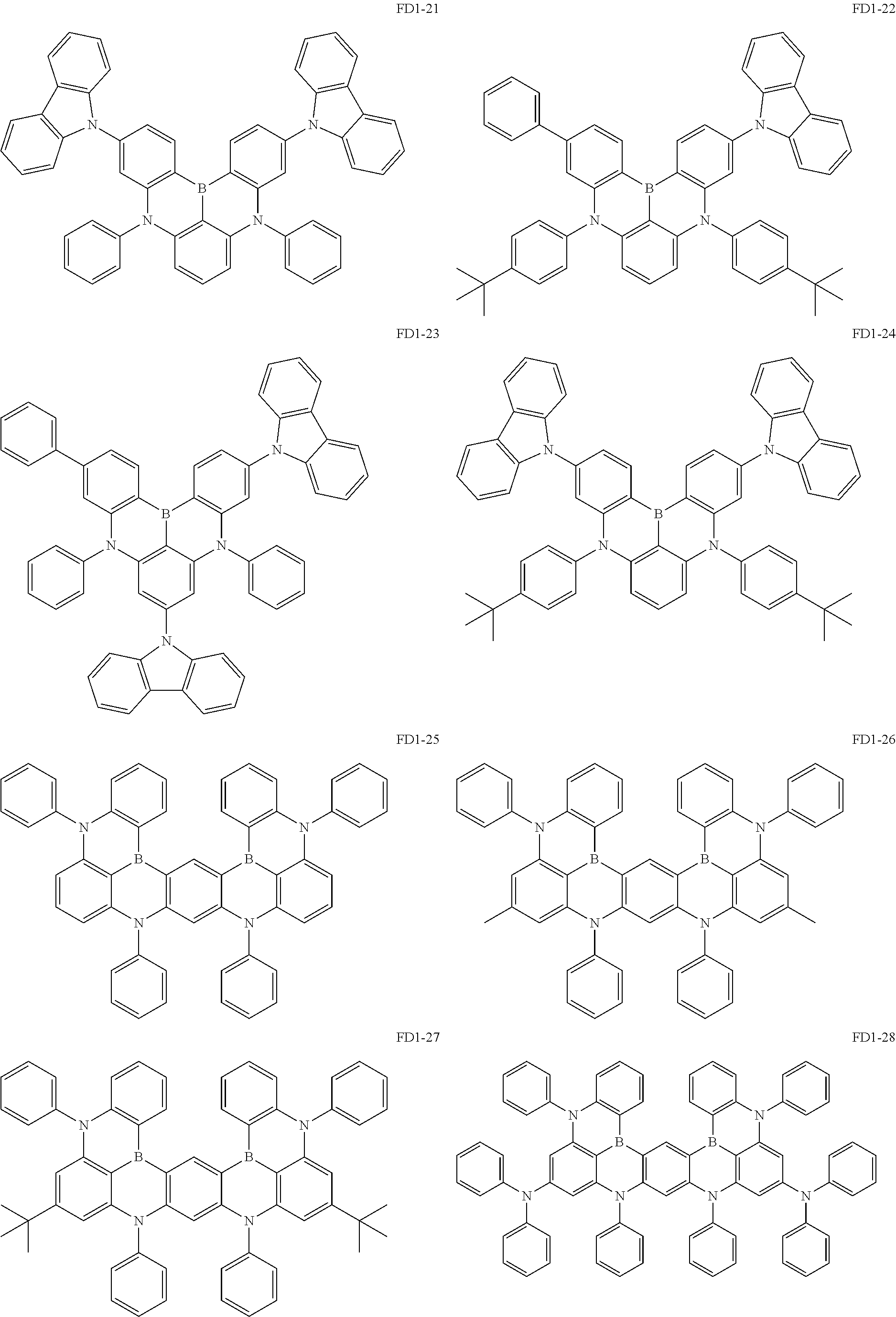

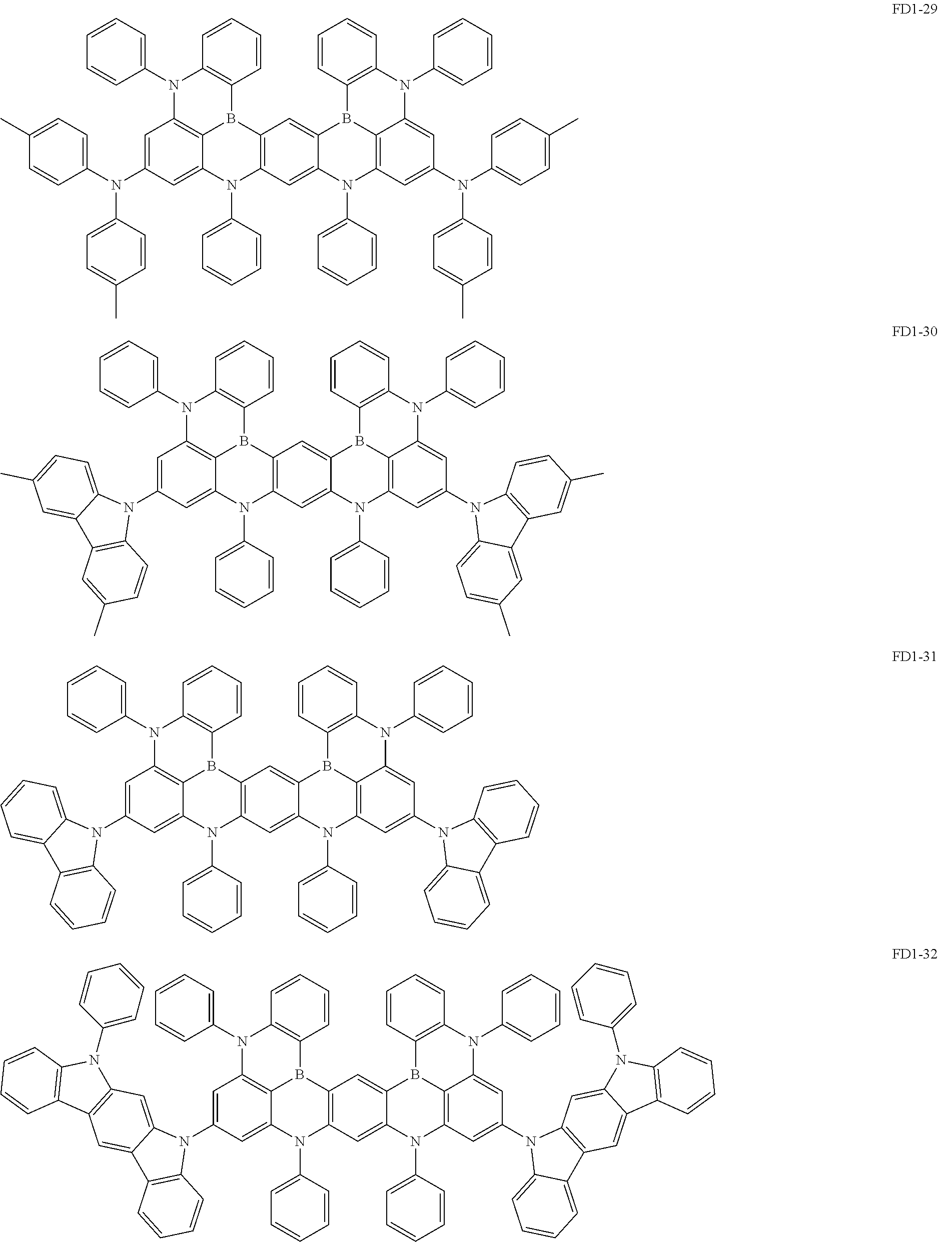

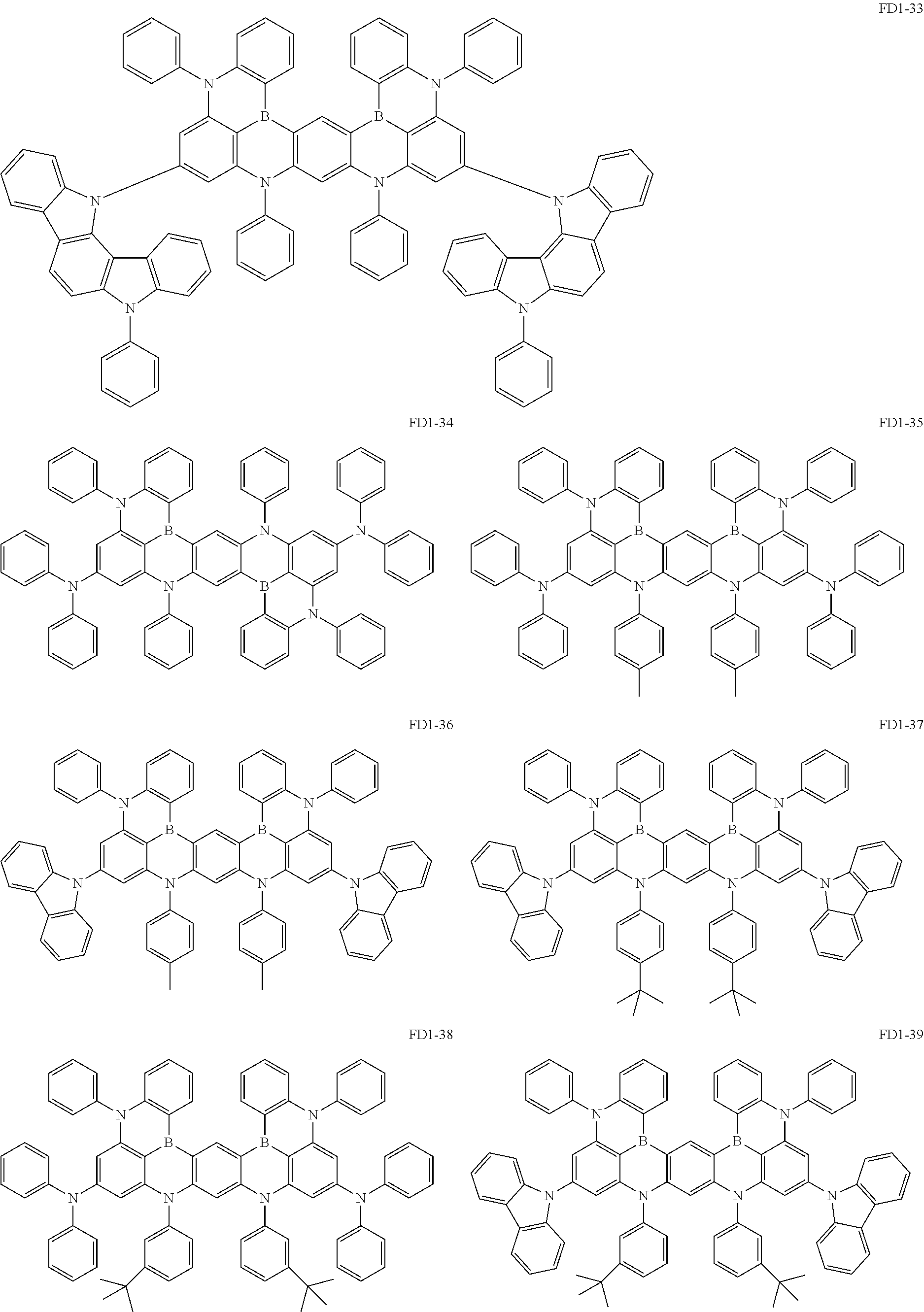

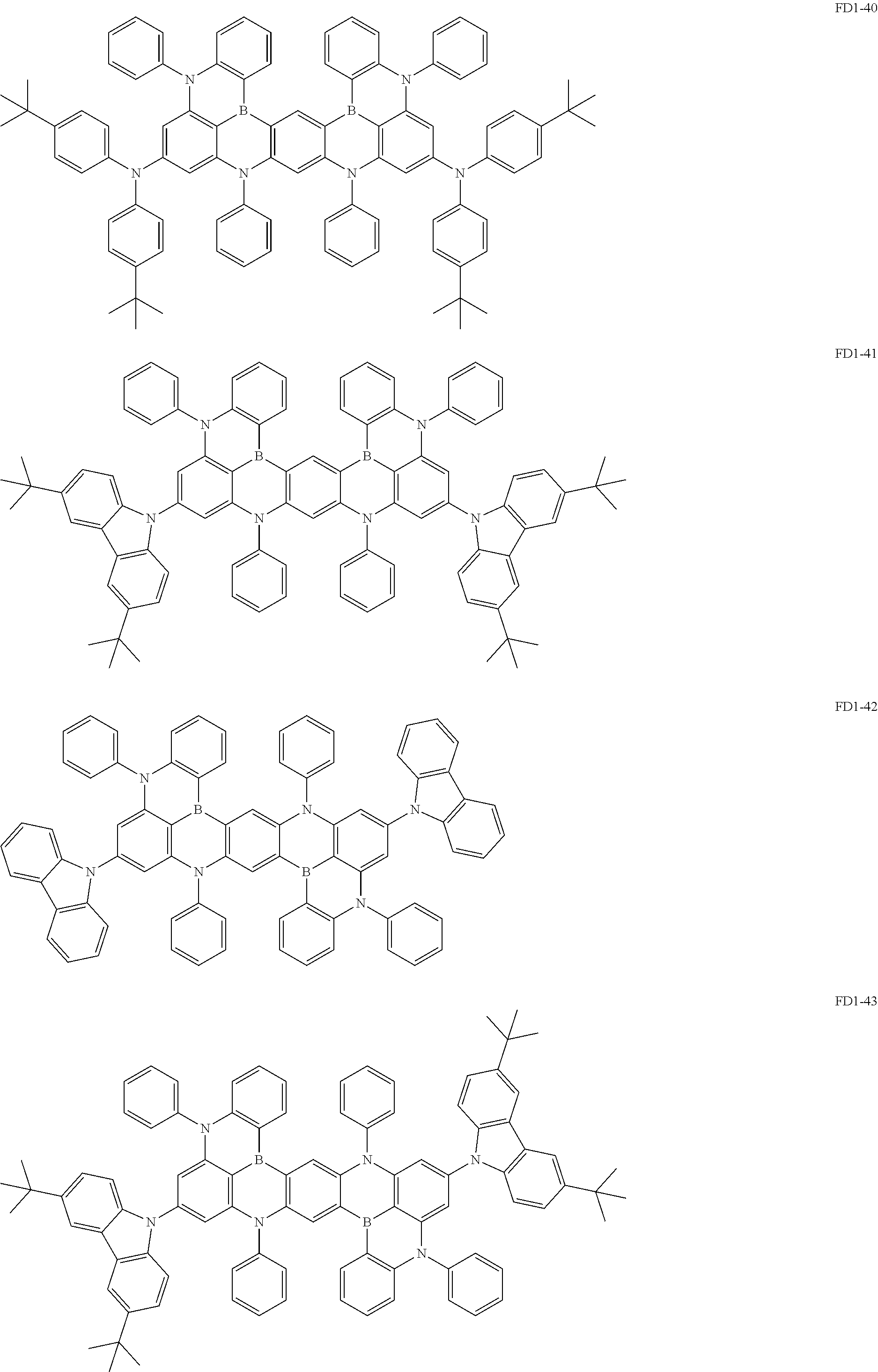

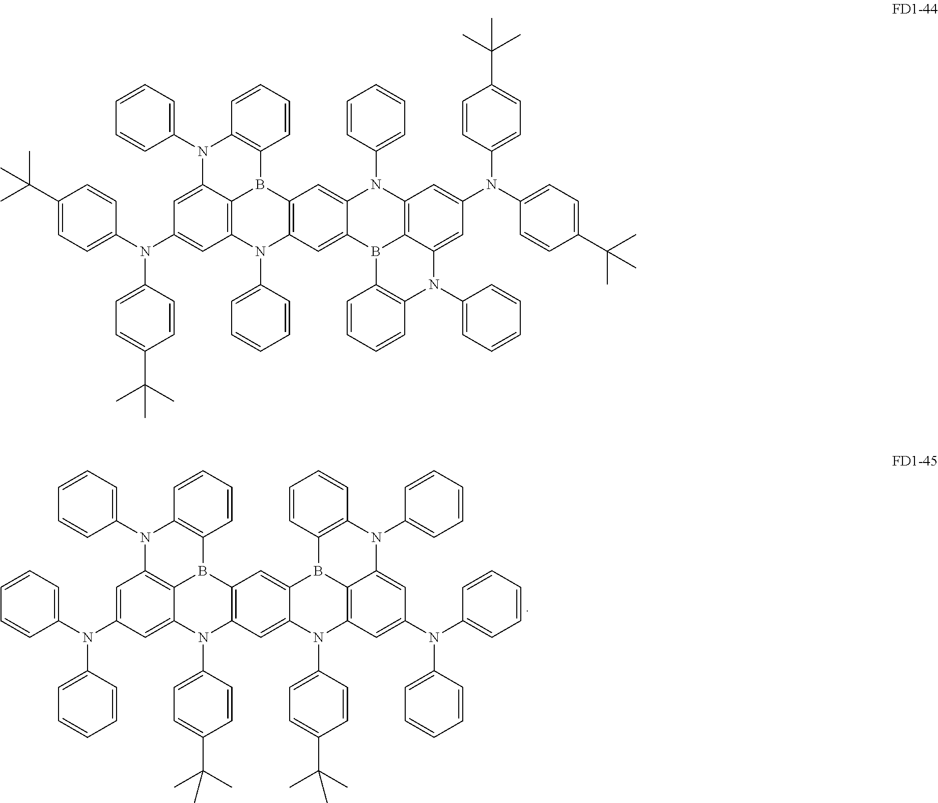 [SMILES: CC(C)(C)c1ccc(N(c2ccc(C(C)(C)C)cc2)c2cc3c4c(c2)N(c2ccccc2)c2cc5c(cc2B4c2ccccc2N3c2ccccc2)N(c2ccccc2)c2cc(N(c3ccc(C(C)(C)C)cc3)c3ccc(C(C)(C)C)cc3)cc3c2B5c2ccccc2N3c2ccccc2)cc1 ; CC(C)(C)c1ccc(N2c3cc4c(cc3B3c5ccccc5N(c5ccccc5)c5cc(N(c6ccccc6)c6ccccc6)cc2c53)B2c3ccccc3N(c3ccccc3)c3cc(N(c5ccccc5)c5ccccc5)cc(c32)N4c2ccc(C(C)(C)C)cc2)cc1]

In another example embodiment, the emitter 346 having fluorescent property can be a pyrene-based organic compound. For example, the pyrene-based organic compound can include an organic compound having the following structure of Chemical Formula 25:

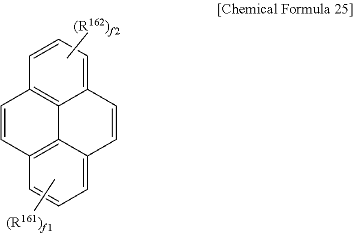

wherein, in the Chemical Formula 25,
each of R¹⁶¹ and R¹⁶² is independently an unsubstituted or substituted C₁-C₂₀ alkyl group, an unsubstituted or substituted C₆-C₃₀ aryl group or an unsubstituted or substituted C₆-C₃₀ aryl amino group, where each R¹⁶¹ is identical to or different from each other when f1 is 2 or 3 and each R¹⁶² is identical to or different from each other when f2 is 2 or 3; and
each of f1 and f2 is independently 0, 1, 2 or 3.

As an example, each of R¹⁶¹ and R¹⁶² in Chemical Formula 25 can be independently a C₆-C₃₀ aryl amino group (e.g., di-phenyl amino) and/or each of f1 and f2 can be 2. For example, the emitter 346 with fluorescent property having the structure of Chemical Formula 25 can be, but is not limited to, at least one of the following organic compounds of Chemical Formula 26:

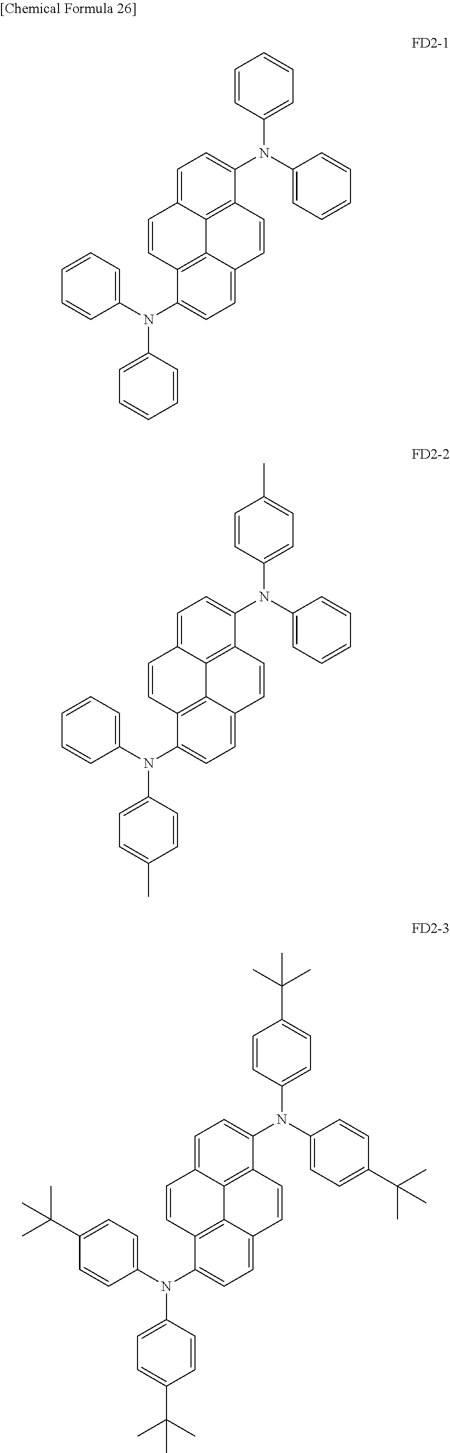 [SMILES: c1ccc(N(c2ccccc2)c2ccc3ccc4c(N(c5ccccc5)c5ccccc5)ccc5ccc2c3c54)cc1 ; Cc1ccc(N(c2ccccc2)c2ccc3ccc4c(N(c5ccccc5)c5ccc(C)cc5)ccc5ccc2c3c54)cc1 ; CC(C)(C)c1ccc(N(c2ccc(C(C)(C)C)cc2)c2ccc3ccc4c(N(c5ccc(C(C)(C)C)cc5)c5ccc(C(C)(C)C)cc5)ccc5ccc2c3c54)cc1]

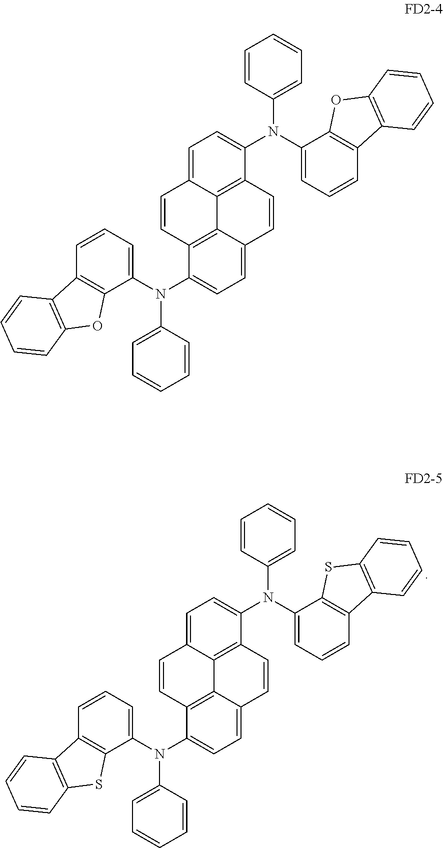 [SMILES: c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

In still another example, the emitter 346 emitting blue color can include, but is not limited to, perylene, 4,4′-Bis[4-(di-p-tolylamino)styryl]biphenyl (DPAVBi), 4-(Di-p-tolylamino)-4-4′-[(di-p-tolylamino)styryl]stilbene (DPAVB), 4,4′-Bis[4-(diphenylamino)styryl]biphenyl (BDAVBi), 2,7-Bis(4-diphenylamino)styryl)-9,9-spiorfluorene (spiro-DPVBi), [1,4-bis[2-[4-[N,N-di(p-tolyl)amino]phenyl]vinyl] benzene (DSB), 1-4-di-[4-(N,N-diphenyl)amino]styryl-benzene (DSA), 2,5,8,11-Tetra-tetr-butylperylene (TBPe), Bis(2-hydroxylphenyl)-pyridine)beryllium (Bepp2), 9-(9-Phenylcarbazole-3-yl)-10-(naphthalene-1-yl)anthracene (PCAN), mer-Tris(1-phenyl-3-methylimidazolin-2-ylidene-C,C(2)′iridium(III) (mer-Ir(pmi)₃), fac-Tris(1,3-diphenyl-benzimidazolin-2-ylidene-C,C(2)′iridium(III) (fac-Ir(dpbic)₃), Bis(3,4,5-trifluoro-2-(2-pyridyl)phenyl-(2-carboxypyridyl)iridium(III) (Ir(tfpd)₂pic), tris(2-(4,6-difluorophenyl)pyridine))iridium(III) (Ir(Fppy)₃), Bis[2-(4,6-difluorophenyl)pyridinato-C²,N](picolinato)iridium(III) (FIrpic) and/or combinations thereof.

When the EML 340 includes both the first and second hosts 342 and 344, the first host 342 and the second host 344 in the EML 340 can be admixed, but is not limited to, a weight ratio between about 4:1 to about 1:4, for example, about 3:1 to about 1:3. As an example, the EML 340 can have a thickness of, but is not limited to, about 100 Å to about 500 Å.

In one example embodiment, the emitter 346 can be one of the above delayed fluorescent material, the phosphorescent material and the fluorescent material. In this case, the contents of the host including the first host 342 and the second host 344 in the EML 340 can be about 50 wt % to about 99 wt %, for example, about 80 wt % to about 95 wt %, and the contents of the emitter 346 in the EML 340 can be about 1 wt % to about 50 wt %, for example, about 5 wt % to about 20 wt %, but is not limited thereto.

In another example embodiment, the emitter 346 can include at least two of the delayed fluorescent material, the phosphorescent material and the fluorescent material. For example, the emitter 346 can include the delayed fluorescent material and the fluorescent material. In this case, the contents of host including the first host 342 and the second host 344 in the EML 340 can be larger than the contents of the delayed fluorescent material and the contents of the delayed fluorescent material in the EML 340 can be larger than the contents of the fluorescent material. For example, the contents of the host including the first and second hosts 342 and 344 in the EML 340 can be about 55 wt % to about 85 wt %, the contents of the delayed fluorescent material in the EML 340 can be about 10 wt % to about 40 wt %, for example, about 10 wt % to about 30 wt %, and the contents of the fluorescent material in the EML 340 can be about 0.1 wt % to about 5 wt %, for example, about 0.1 wt % to about 2 wt %, but is not limited thereto.

Alternatively, the emitter 346 can include the phosphorescent material and the fluorescent material. In this case, the EML 340 can be phospho-sensitized-fluorescence (PSF) emitting material layer. The contents of the host including the first and second host 342 and 344 in the EML 340 can be larger than the contents of the phosphorescent material and the contents of the phosphorescent material in the EML 340 can be larger than the contents of the fluorescent material. As an example, the contents of the host including the first and second hosts 342 and 344 in the EML 340 can be about 3000 parts to about 6000 parts by weight, and the contents of the phosphorescent material in the EML 340 can be about 500 parts to about 200 parts by weight based on 100 parts by weight of the fluorescent material.

The ETL 360 and the EIL 370 can be laminated sequentially between the EML 340 and the second electrode 230. An electron transporting material included in the ETL 360 has high electron mobility so as to provide electrons stably with the EML 340 by fast electron transportation.

In one example embodiment, the ETL 360 can include at least one of an oxadiazole-based compound, a triazole-based compound, a phenanthroline-based compound, a benzoxazole-based compound, a benzothiazole-based compound, a benzimidazole-based compound, a triazine-based compound and/or combinations thereof.

More particularly, the ETL 360 can include, but is not limited to, tris-(8-hydroxyquinoline aluminum (Alq₃), 2-biphenyl-4-yl-5-(4-t-butylphenyl)-1,3,4-oxadiazole (PBD), spiro-PBD, lithium quinolate (Liq), 1,3,5-Tris(N-phenylbenzimidazol-2-yl)benzene (TPBi), Bis(2-methyl-8-quinolinolato-N1,O8)-(1,1′-biphenyl-4-olato)aluminum (BAlq), 4,7-diphenyl-1,10-phenanthroline (Bphen), 2,9-Bis(naphthalene-2-yl)4,7-diphenyl-1,10-phenanthroline (NBphen), 2,9-Dimethyl-4,7-diphenyl-1,10-phenathroline (BCP), 3-(4-Biphenyl)-4-phenyl-5-tert-butylphenyl-1,2,4-triazole (TAZ), 4-(Naphthalen-1-yl)-3,5-diphenyl-4H-1,2,4-triazole (NTAZ), 1,3,5-Tri(p-pyrid-3-yl-phenyl)benzene (TpPyPB), 2,4,6-Tris(3′-(pyridin-3-yl)biphenyl-3-yl)1,3,5-triazine (TmPPPyTz), Poly[9,9-bis(3′-((N,N-dimethyl)-N-ethylammonium)-propyl)-2,7-fluorene]-alt-2,7-(9,9-dioctylfluorene)] (PFNBr), tris(phenylquinoxaline (TPQ), TSPO1, 2-[4-(9,10-Di-2-naphthalen2-yl-2-anthracen-2-yl)phenyl]-1-phenyl-1H-benzimidazole (ZADN) and/or combinations thereof:

The EIL 370 is disposed between the second electrode 230 and the ETL 360, and can improve physical properties of the second electrode 230 and therefore, can enhance the lifespan of the OLED D1. In one example embodiment, the EIL 370 can include, but is not limited to, an alkali metal halide or an alkaline earth metal halide such as LiF, CsF, NaF, BaF₂ and the like, and/or an organometallic compound such as Liq, lithium benzoate, sodium stearate, and the like. Alternatively, the EIL 370 can be omitted.

When holes are transferred to the second electrode 230 via the EML 340 and/or electrons are transferred to the first electrode 210 via the EML 340, the OLED D1 can have short lifespan and reduced luminous efficiency. In order to prevent those phenomena, the OLED D1 in accordance with this aspect of the present disclosure can have at least one exciton blocking layer adjacent to the EML 340.

As an example, the OLED D1 can include the EBL 330 between the HTL 320 and the EML 340 so as to control and prevent electron transfers. In one example embodiment, the EBL 330 can include, but is not limited to, TCTA, Tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, mCP, mCBP, CuPc, DNTPD, TDAPB, DCDPA, 2,8-bis(9-phenyl-9H-carbazol-3-yl)dibenzo[b,d]thiophene and/or combinations thereof.

In addition, the OLED D1 can further include the HBL 350 as a second exciton blocking layer between the EML 340 and the ETL 360 so that holes cannot be transferred from the EML 340 to the ETL 360. In one example embodiment, the HBL 350 can include, but is not limited to, at least one of an oxadiazole-based compound, a triazole-based compound, a phenanthroline-based compound, a benzoxazole-based compound, a benzothiazole-based compound, a benzimidazole-based compound, a triazine-based compound and/or combinations thereof.

For example, the HBL 350 can include material having a relatively low HOMO energy level compared to the luminescent materials in EML 340. The HBL 350 can include, but is not limited to, BCP, BAlq, Alq₃, PBD, spiro-PBD, Liq, Bis-4,5-(3,5-di-3-pyridylphenyl)-2-methylpyrimidine (B3PYMPM), DPEPO, 9-(6-(9H-carbazol-9-yl)pyridine-3-yl)-9H-3,9′-bicarbazole, TSPO1 and/or combinations thereof.

As described above, the EML 340 includes hosts 342 and/or 344 and an emitter 346, and the first host 342 includes an organic compound having the structure of Chemical Formulae 1 to 8. As the electron donor moiety of the carbazolyl moiety is enhanced, the delayed fluorescent property of the organic compound can be improved. As excitons are generated via both ISC and RISC, and thus the organic compound can prevent decrease in luminous efficiency owing to non-emitting excitons and can have improved stability to holes leaked from other luminous materials.

In addition, it is possible to minimize or reduce reduction in luminescence lifetime due to thermal decomposition of other substituents linked to the carbazolyl moiety, and to prevent decrease in singlet and/or triplet energy level due to expansion of a conjugated structure.

The organic compound further includes a blocking moiety of an aromatic and/or hetero aromatic structure linked to the triazine moiety. The organic compound can maintain high triplet energy level, can have beneficial electron transporting properties and can have limited molecular packing as the exciton binding energy decrease. In addition, the organic compound has bipolar property by including the triazine moiety as an electron donor and the carbazolyl moiety as an electron acceptor.

Therefore, the organic compound can be applied to the emissive layer of an organic light emitting diode. For example, the organic compound has a higher singlet energy level S₁ and a higher triplet energy level T₁ and a wider energy bandgap between HOMO energy level and LUMO energy level compared to the emitter. Exciton energy can be rapidly transferred to the emitter in the emitting material layer by applying the organic compound as hosts of the emitting material layer.

An organic light emitting diode can include two or more emitting parts. FIG. 4 illustrates a cross-sectional view of an organic light emitting diode having a single emitting part in accordance with another example embodiment of the present disclosure. As illustrated in FIG. 4, the organic light emitting diode (OLED) D2 includes a first electrode 210, a second electrode 230 facing the first electrode 210 and an emissive layer 220A disposed between the first and second electrodes 210 and 230. The organic light emitting display device 100 includes a red pixel region, a green pixel region and a blue pixel region, and the OLED D2 can be disposed in the red pixel region, the green pixel region and the blue pixel region. As an example, the OLED D2 can be disposed in the blue pixel region. The first electrode 210 can be an anode and the second electrode 230 can be a cathode, but is not limited thereto.

The emissive layer 220A includes a first emitting part 300 and a second emitting part 400. The emissive layer 200A can further include a charge generation layer (CGL) 380 disposed between the first emitting part 300 and the second emitting part 400 so that the first emitting part 300, the CGL 380 and the second emitting part 400 are stacked sequentially between the first electrode 210 and the second electrode 230. In other words, the first emitting part 300 is disposed between the first electrode 210 and the CGL 380, and the second emitting part 400 is disposed between the CGL 380 and the second electrode 230.

The first emitting part 300 includes a first emitting material layer (lower emitting material layer, EML1) 340. The first emitting part 300 can further include at least one of a hole injection layer (HIL) 310 disposed between the fist electrode 210 and the EML1 340, a first hole transport layer (HTL1) 320 disposed between the HIL 310 and the EML1 340 and a first electron transport layer (ETL1) 360 disposed between the EML1 340 and the CGL 380. Alternatively, the first emitting part 300 can further include at least one of a first electron blocking layer (EBL1) 330 disposed between the HTL1 320 and the EML1 340 and a first hole blocking layer (HBL1) 350 disposed between the EML1 340 and the ETL1 360.

The second emitting part 400 includes a second emitting material layer (upper emitting material layer, EML2) 440. The second emitting part 400 can further include at least one of a second hole transport layer (HTL2) 420 disposed between the CGL 380 and the EML2 440, a second electron transport layer (ETL2) 460 disposed between the EML2 440 and the second electrode 230 and an electron injection layer (EIL) 470 disposed between the ETL2 460 and the second electrode 230. Alternatively, the second emitting part 400 can further at least one of a second electron blocking layer (EBL2) 430 disposed between the HTL2 420 and the EML2 440 and a second hole blocking layer (HBL2) 450 disposed between the EML2 440 and the ETL2 460.

The materials of the HIL 310, the HTL1 320 and the HTL2 420, the EBL1 330 and the EBL2 430, the HBL1 350 and the HBL2 450, the ETL1 360 and the ETL2 460 and the EIL 470 can be identical to the corresponding materials with referring to FIG. 3

The CGL 380 is disposed between the first emitting part 300 and the second emitting part 400. The first emitting part 300 and the second emitting part 400 are connected by the CGL 380. The CGL 380 can be PN-junction charge generation layer by connected by an N-type charge generation layer (N-CGL) 382 and a P-type charge generation layer (P-CGL) 384.

The N-CGL 382 is disposed between the ETL1 360 and the HTL2 420 and the P-CGL 384 is disposed between the N-CGL 382 and the HTL2 420. The N-CGL 382 provides electrons to the EML1 340 of the first emitting part 300 and the P-CGL 384 provides holes to the EML2 440 of the second emitting part 400.

For example, the N-CGL 382 can include electron transporting material doped with an alkali metal (e.g., Li, Na, K, Rb and Cs) and/or an alkaline earth metal (e.g., Mg, Ca, Sr, Ba and Ra). The contents of the alkali metal and/or the alkaline earth metal in the N-CGL 382 can be, but is not limited to, between about 1 wt % and about 10 wt %. As an example, the P-CGL 384 can include hole transporting material doped with hole injecting material (e.g., HAT-CN, F4-TCNA and/or F6-TCNNQ). The contents of the hole injecting material in the P-CGL 384 can be, but is not limited to, between about 2 wt % and about 15 wt %.

In one example embodiment, both the EML1 340 and the EML2 440 can be a blue emitting material layer. For example, the EML1 340 includes a first host 342 having the structure of Chemical Formulae 1 to 8 of an N-type host, and optionally, a second host 344 of a P-type host and/or an emitter 346 which can be at least one of delayed fluorescent material, phosphorescent material and fluorescent material.

Also, the EML2 440 includes a first host 442 having the structure of Chemical Formulae 1 to 8 as an N-type host, and optionally, a second host 444 of a P-type host and/or an emitter 446 which can be at least one of delayed fluorescent material, phosphorescent material and fluorescent material.

Each of the first host 342, the second host 344 and the emitter 346 in the EML1 340 can be independently identical to or different from the first host 442, the second host 444 and the emitter 446 in the EML2 440, respectively. The contents of the luminous materials in each of the EML1 340 and the EML2 440 can be identical to the contents of the corresponding materials with referring to FIG. 3

Alternatively, the EML2 440 include materials different from at least one of the first host 342, the second host 344 and the emitter 346 in the EML1 340 so that the EML2 440 can emit color different from the EML1 340 or can have luminous efficiency different from the EML1 340.

At least one of the emitting parts 300 and 400 in the OLED D2 includes the organic compound having the structure of Chemical Formulae 1 to 8. As at least one of the first emitting part 300 and the second emitting part 400 includes the organic compound with enhanced delayed fluorescent property, it is possible to suppress non-emissive excitons and improve stability to holes, to minimize decrease in luminous lifetime and/or to prevent decrease of energy levels. The organic compound having high singlet and/or triplet energy levels and wide energy bandgap compared to the emitter is applied into the emissive layer 220A, and thus, the organic compound can transfer exciton energy to the emitter. In addition, the OLED D2 can have beneficial color sense and optimized luminous efficiency since the OLED D2 has a dual stack structure of two emitting material layers.

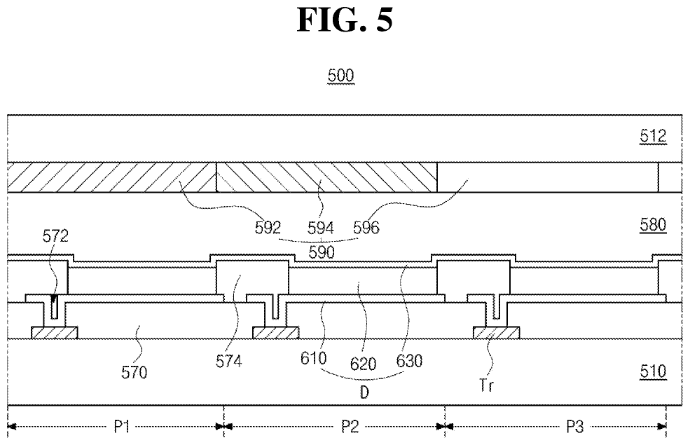

The organic light emitting device and the OLED D1 and the OLED D2 with a single emitting part or a plurality of emitting parts are shown in FIGS. 2 to 4. In another example embodiment, an organic light emitting display device can implement full-color including white color. FIG. 5 illustrates a schematic cross-sectional view of an organic light emitting display device in accordance with another example embodiment of the present disclosure.

As illustrated in FIG. 5, the organic light emitting display device 500 includes a first substrate 510 that defines each of a first pixel region P1, a second pixel region P2 and a third pixel region P3, a second substrate 512 facing the first substrate 510, a thin film transistor Tr on the first substrate 510, an OLED D disposed between the first and second substrates 510 and 512 and emitting white (W) light and a color filter layer 590 disposed between the OLED D and the second substrate 512. For example, each of the first to third pixel regions P1, P2 and P3 can be a red pixel region, a green pixel region and a blue pixel region, respectively. Alternatively, the first substrate 510 can further include a fourth pixel region of a white pixel region.

Each of the first and second substrates 510 and 512 can include, but is not limited to, glass, flexible material and/or polymer plastics. For example, each of the first and second substrates 510 and 512 can be made of PI, PES, PEN, PET, PC and/or combinations thereof. The first substrate 510, on which a thin film transistor Tr and the OLED D are arranged, forms an array substrate. The second substrate 512 can be omitted.

The thin film transistor Tr can be disposed on the first substrate 510. Alternatively, a buffer layer is disposed on the first substrate 510 and the thin film transistor Tr can be disposed on the buffer layer. As illustrated in FIG. 2, the thin film transistor can include a semiconductor layer, a gate electrode, a source electrode and a drain electrode and acts as a driving element.

A passivation layer 570 is disposed on the thin film transistor Tr. The passivation layer 570 has a flat top surface and has a drain contact hole 572 that exposes or does not cover the drain electrode of the thin film transistor Tr.

The OLED D is located on the passivation layer 570 correspondingly to the color filter layer 590. The OLED D includes a first electrode 610 that is connected to the drain electrode of the thin film transistor Tr, and an emissive layer 620 and a second electrode 630 disposed sequentially on the first electrode 610. The OLED D emits white color light in the first to third pixel regions P1, P2 and P3.

The first electrode 610 is formed for each pixel region P1, P2 or P3 and the second electrode 630 is formed integrally corresponding to the first to third pixel regions P1, P2 and P3. The first electrode 610 can be one of an anode and a cathode and the second electrode 620 can be the other of the anode and the cathode. In one example embodiment, the first electrode 610 can be a reflective electrode and the second electrode 620 can be a transmissive (or semi-transmissive) electrode. Alternatively, the first electrode 610 can be the transmissive (or semi-transmissive) electrode and the second electrode 620 can be the reflective electrode.

For example, the first electrode 610 can be the anode, and can include a conductive material having relatively high work function value, for example, transparent conductive oxide (TCO). As an example, the first electrode 610 can include, but is not limited to, ITO, IZO, ITZO, SnO, ZnO, ICO, AZO, and/or combinations thereof. Alternatively, a reflective electrode or a reflective layer can be disposed under the first electrode 610.

The second electrode 630 can be the cathode, and can include a conductive material having relatively low work function value, for example, low resistant metal. For example, the second electrode 620 can include, but is not limited to, Al, Mg, Ca, Ag, alloy thereof (e.g., Mg—Al alloy) and/or combinations thereof.

In one example embodiment, as the light emitted from the emissive layer 620 can be incident to the color filter layer 590 through the second electrode 620 in the organic light emitting display device 500, the second electrode 620 can have a thin thickness to transmit the light emitted from the emissive layer 620.

An emissive layer 630 can include at least two emitting parts each of which emits different color light. Each emitting part can have single-layered structure of an emitting material layer (EML). Alternatively, each emitting part can further comprise at least one of a hole injection layer (HIL), a hole transport layer (HTL), an electron blocking layer (EBL), a hole blocking layer (HBL), an electron transport layer (ETL) and an electron injection layer (EIL). In addition, the emissive layer 620 can further include at least one charge generation layer (CGL) disposed between two emitting parts.

A bank layer 574 is disposed on the passivation layer 570 in order to cover edges of the first electrode 610. The bank layer 574 exposes or does not cover a center of the first electrode 610 corresponding to each of the first to third pixel regions P1, P2 and P3. The bank layer 574 is formed to prevent current leakage at the edge of the first electrode 610. The bank layer 574 can be omitted.

Since the OLED D emits white color light in the first to third pixel regions P1, P2 and P3, the emissive layer 620 can be formed as a common layer without being separated from in the first to third pixel regions P1, P2 and P3.

The organic light emitting display device 500 can further include an encapsulation film 580 that can be disposed on the second electrode 630 in order to prevent or reduce outer moisture from penetrating into the OLED D. In addition, a polarizing plate can be attached under the first substrate 510 or onto the second substrate 512 to reduce reflection of external light.

The color filter layer 590 is disposed on the OLED D or the encapsulation film 580. For example, the color filter layer 590 can include a first color filter layer 592 corresponding to the first pixel region P1, a second color filter layer 594 corresponding to the second pixel region P2 and a third color filter layer 596 corresponding to the third pixel region P3. For example, the first color filter layer 592 can be a red color filter layer, the second color filter layer 594 can be a green color filter layer and the third color filter layer 596 can be a blue color filter layer.

For example, the first color filter layer 592 can include at least one of red dye and blue pigment, the second color filter layer 594 can include at least one of green dye and green pigment and the third color filter layer 596 can include at least one of blue dye and blue pigment. In one example embodiment, the color filter layer 590 can be attached to the OLED D through an adhesive layer. Alternatively, the color filter layer 590 can be disposed directly on the OLED D.

In FIG. 5, the light emitted from the OLED D is transmitted through the second electrode 620 and the color filter layer 590 is disposed on the OLED D. In this case, the organic light emitting display device 500 can be a top-emission type. Alternatively, when the organic light emitting display device 500 is a bottom-emission type, the light emitted from the OLED D is transmitted through the first electrode 610 and the color filter layer 590 can be disposed between the OLED D and the first substrate 510.

In addition, a color conversion layer may be formed or disposed between the OLED D and the color filter layer 590. The color conversion layer may include a red color conversion layer, a green color conversion layer and a blue color conversion layer each of which is disposed correspondingly to each pixel region P1, P2 or P3, so as to convert the white (W) color light to each of a red, green and blue color lights, respectively. Alternatively, the organic light emitting display device 500 can comprise the color conversion film instead of the color filter layer 590.

As described above, the white (W) color light emitted from the OLED D is transmitted through the first to third color filter layers 592, 594 and 596 each of which is disposed correspondingly to the first to third pixel regions P1, P2 and P3, respectively, so that red, green and blue color lights are displayed in the first to third pixel regions P1, P2 and P3.

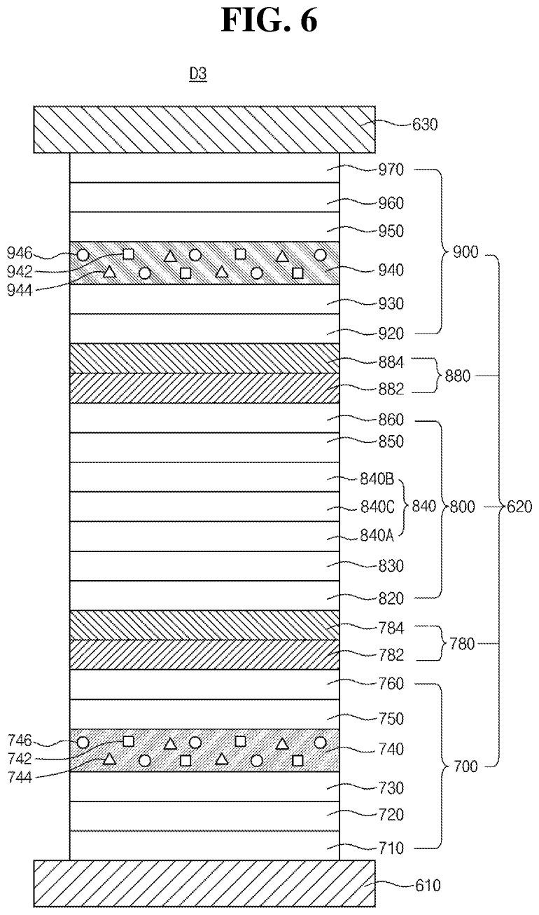

An OLED that can be applied into the organic light emitting display device will be described in more detail. FIG. 6 illustrates a schematic cross-sectional view of an organic light emitting diode having a tandem structure of two emitting parts.

As illustrated in FIG. 6, the OLED D3 in accordance with the example embodiment of the present disclosure includes first and second electrodes 610 and 630 facing each other and an emissive layer 620 disposed between the first and second electrodes 610 and 630. The emissive layer 620 includes a first emitting part 700 disposed between the first electrode 610 and the second electrode 630, a second emitting part 800 disposed between the first emitting part 700 and the second electrode 630, a third emitting part 900 disposed between the second emitting part 800 and the second electrode 630, a first charge generation layer (CGL1) 780 disposed between the first emitting part 700 and the second emitting part 800, and a second charge generation layer (CGL2) 880 disposed between the second emitting part 800 and the third emitting part 900.

The first emitting part 700 includes a first emitting material layer (lower emitting material layer, EML1) 740. The first emitting part 700 can further include at least one of a hole injection layer (HIL) 710 disposed between the first electrode 610 and the EML1 740, a first hole transport layer (HTL1) 720 disposed between the HIL 710 and the EML1 740, and a first electron transport layer (ETL1) 760 disposed between the EML1 740 and the CGL1 780. Alternatively, the first emitting part 700 can further include at least one of a first electron blocking layer (EBL1) 730 disposed between the HTL1 720 and the EML1 740 and a first hole blocking layer (HBL1) 750 disposed between the EML1 740 and the ETL1 760.

The second emitting part 800 includes a second emitting material layer (middle emitting material layer, EML2) 840. The second emitting part 800 can further include at least one of a second hole transport layer (HTL2) 820 disposed between the CGL1 780 and the EML2 840 and a second electron transport layer (ETL2) 860 disposed between the EML2 840 and the CGL2 880. Alternatively, the second emitting part 800 can further include at least one of a second electron blocking layer (EBL2) 830 disposed between the HTL2 820 and the EML2 840 and a second hole blocking layer (HBL2) 850 disposed between the EML2 840 and the ETL2 860.

The third emitting part 900 includes a third emitting material layer (upper emitting material layer, EML3) 940. The third emitting part 900 can further include at least one of a third hole transport layer (HTL3) 920 disposed between the CGL2 880 and the EML3 940, a third electron transport layer (ETL3) 960 disposed between the EML3 940 and the second electrode 630, and an electron injection layer (EIL) 970 disposed between ETL3 960 and the second electrode 630. Alternatively, the third emitting part 900 can further include a third electron blocking layer (EBL3) 930 disposed between the HTL3 920 and the EML3 940 and a third hole blocking layer (HBL3) 950 disposed between the EML3 940 and the ETL3 960.

The CGL1 780 is disposed between the first emitting part 700 and the second emitting part 800 and the CGL2 880 is disposed between the second emitting part 800 and the third emitting part 900. The CGL1 780 includes a first N-type charge generation layer (N-CGL1) 782 disposed between the ETL1 760 and the HTL2 820 and a first P-type charge generation layer (P-CGL1) 784 disposed between the N-CGL1 782 and the HTL2 820. The CGL2 880 includes a second N-type charge generation layer (N-CGL2) 882 disposed between the ETL2 860 and the HTL3 920 and a second P-type charge generation layer (P-CGL2) 884 disposed between the N-CGL2 882 and the HTL3 920.

Each of the N-CGL1 782 and the N-CGL2 882 provides electrons to the EML1 740 of the first emitting part 700 and the EML2 840 of the second emitting part 800, respectively. Each of the P-CGL1 784 and the P-CGL2 884 provides holes to the EML2 840 of the second emitting part 800 and the EML3 940 of the third emitting part 900, respectively.

The materials of the HIL 710, the HTL1 to HTL3 720, 820 and 920, the EBL1 to EBL3 730, 830 and 930, the HBL1 to HBL3 750, 850 and 950, the ETL1 to ETL3 760, 860 and 960, the EIL 970, the CGL1 780 and the CGL2 880 can be identical to the corresponding materials with referring to FIGS. 3 and 4.

At least one of the EML1 740, the EML2 840 and the EML3 940 can include the organic compound having the structure of Chemical Formulae 1 to 8. For example, at least one of the EML1 740, the EML2 840 and the EML3 940 can emit blue color light and the other of the EML1 740, the EML2 840 and the EML3 940 can emit red to green color light, so that the OLED D3 can realize white (W) emission. Hereinafter, the OLED D3 where the EML1 740 and/or the EML3 940 include the organic compound having the structure of Chemical Formulae 1 to 8 to emit blue color light, and the EML2 840 emits red to green color light will be described in detail.

In one example embodiment, each of the EML1 740 and the EML3 940 can be a blue emitting material layer. For example, the EML1 740 can include a first host 742 having the structure of Chemical Formulae 1 to 8 of an N-type host, and optionally, a second host 744 of a P-type host and/or an emitter which can be at least one of delayed fluorescent material, phosphorescent material and fluorescent material.

Also, the EML3 940 includes a first host 942 having the structure of Chemical Formulae 1 to 8 as an N-type host, and optionally, a second host 944 of a P-type host and/or an emitter 946 which can be at least one of delayed fluorescent material, phosphorescent material and fluorescent material.

Each of the first host 742, the second host 744 and the emitter 346 in the EML1 740 can be independently identical to or different from the first host 942, the second host 944 and the emitter 946 in the EML3 940, respectively. The contents of the luminous materials in each of the EML1 740 and the EML3 940 can be identical to the contents of the corresponding materials with referring to FIGS. 3 and 4.

Alternatively, the EML3 940 include materials different from at least one of the first host 742, the second host 744 and the emitter 746 in the EML1 740 so that the EML3 940 can emit color different from the EML1 740 or can have luminous efficiency different from the EML1 740.

The EML2 840 can include a first layer 840A disposed between the EBL2 830 and the HBL2 850, a second layer 840B disposed between the first layer 840A and the HBL2 850, and optionally, a third layer 840C disposed between the first layer 840A and the second layer 840B. In one example embodiment, one of the first layer 840A and the second layer 840B can emit red color light and the other of the first layer 840A and the second layer 840B can emit green color light. Hereinafter, the EML2 840 where the first layer 840A emits a red color light and the second layer 840B emits a green color light will be described in detail.

The first layer 840A includes a red host and a red emitter (red dopant). As an example, the red host can include at least one of a P-type red host and an N-type red host. For example, the red host can include, but is not limited to, mCP-CN, CBP, mCBP, mCP, DPEPO, PPT, TmPyPB, PYD-2Cz, DCzDBT, DCzTPA, pCzB-2CN, mCzB-2CN, TSPO1, CCP, 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicabazole, BCzPh, BCZ, TCP, TCTA, CDBP, DMFL-CBP, Spiro-CBP, TCzl and/or combinations thereof.

The red emitter can include at least one of red phosphorescent material, red fluorescent material and red delayed fluorescent material. As an example, the red emitter can include, but is not limited to, Bis[2-(4,6-dimethyl)phenylquinoline)](2,2,6,6-tetramethylheptane-3,5-dionate)iridium(III), Bis[2-(4-n-hexylphenyl)quinoline](acetylacetonate)iridium(III) (Hex-Ir(phq)₂(acac)), Tris[2-(4-n-hexylphenyl)quinoline]iridium(III) (Hex-Ir(phq)₃), Tris[2-phenyl-4-methylquinoline]iridium(III) (Ir(Mphq)₃), Bis(2-phenylquinoline)(2,2,6,6-tetramethylheptane-3,5-dionate)iridium(III) (Ir(dpm)PQ₂), Bis(phenylisoquinoline)(2,2,6,6-tetramethylheptane-3,5-dionate)iridium(III) (Ir(dpm)(piq)₂), Bis(1-phenylisoquinoline)(acetylacetonate)iridium(III) (Ir(piq)₂(acac)), Bis[(4-n-hexylphenyl)isoquinoline](acetylacetonate)iridium(III) (Hex-Ir(piq)₂(acac)), Tris[2-(4-n-hexylphenyl)quinoline]iridium(III) (Hex-Ir(piq)₃), Tris(2-(3-methylphenyl)-7-methyl-quinolato)iridium (Ir(dmpq)₃), Bis[2-(2-methylphenyl)-7-methyl-quinoline](acetylacetonate)iridium(III) (Ir(dmpq)₂(acac)), Bis[2-(3,5-dimethylphenyl)-4-methyl-quinoline](acetylacetonate)iridium(III) (Ir(mphmq)₂(acac)), Tris(dibenzoylmethane)mono(1,10-phenanthroline)europium(III) (Eu(dbm)₃(phen)) and/or combinations thereof.

For example, the contents of the red host in the first layer 840A can be about 50 wt % to about 99 wt %, for example, about 80 wt % to about 95 wt %, and the contents of the red emitter in the first layer 840A can be about 1 wt % to about 50 wt %, for example, about 5 wt % to about 20 wt %, but is not limited thereto. When the first layer 840A includes both the P-type red host and the N-type red host, the P-type red host and the N-type red host can be admixed, but is not limited to, with a weight ratio of about 4:1 to about 1:4, for example about 3:1 to about 1:3.

The second layer 840B can include a green host and a green emitter (green dopant). As an example, the green host can include at least one of a P-type green host and an N-type green host. In one example embodiment, the green host can be identical to the red host above. Alternatively, the red host can include, but is not limited to, a biscarbazole-based organic compound, an aryl amine- or hetero aryl amine-based organic compound having at least one fused aromatic and/or fused hetero aromatic moiety, and/or an aryl amine- or hetero aryl amine-based organic compound having at least one spirofluorene moiety.

For example, the green host can include, but is not limited to, mCP-CN, CBP, mCBP, mCP, DPEPO, PPT, TmPyPB, PYD-2Cz, DCzDBT, DCzTPA, pCzB-2CN, mCzB-2CN, TSPO1, CCP, 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicabazole, BCzPh, BCZ, TCP, TCTA, CDBP, DMFL-CBP, Spiro-CBP, TCzl and/or combinations thereof.

The green emitter can include at least one of green phosphorescent material, green fluorescent material and green delayed fluorescent material. As an example, the green emitter can include, but is not limited to, [Bis(2-phenylpyridine)](pyridyl-2-benzofuro[2,3-b]pyridine)iridium, Tris[2-phenylpyridine]iridium(III) (Ir(ppy)₃), fac-Tris(2-phenylpyridine)iridium(III) (fac-Ir(ppy)₃), Bis(2-phenylpyridine)(acetylacetonate)iridium(III) (Ir(ppy)₂(acac)), Tris[2-(p-tolyl)pyridine]iridium(III) (Ir(mppy)₃), Bis(2-(naphthalene-2-yl)pyridine)(acetylacetonate)iridium(III) (Ir(npy)₂acac), Tris(2-phenyl-3-methyl-pyridine)iridium (Ir(3mppy)₃), fac-Tris(2-(3-p-xylyl)phenyl)pyridine iridium(III) (TEG) and/or combinations thereof.

The contents of the green host in the second layer 840B can be about 50 wt % to about 99 wt %, for example, about 80 wt % to about 95 wt %, and the contents of the green emitter in the second layer 840B can be about 1 wt % to about 50 wt %, for example, about 5 wt % to about 20 wt %, but is not limited thereto. When the second layer 840B includes both the P-type green host and the N-type green host, the P-type green host and the N-type green host can be admixed, but is not limited to, with a weight ratio of about 4:1 to about 1:4, for example about 3:1 to about 1:3.

The third layer 840C can be a yellow-green emitting material layer. The third layer 840C can include a yellow-green host and a yellow-green emitter (yellow-green dopant). The yellow-green host can include at least one of a P-type yellow-green host and an N-type yellow green host. As an example, the yellow-green host can be identical to the red host above and/or the green host above.

The yellow-green emitter can include at least one of yellow-green phosphorescent material, yellow-green fluorescent material and yellow-green delayed fluorescent material. For example, the yellow-green emitter can include, but is not limited to, 5,6,11,12-Tetraphenylnaphthalene (Rubrene), 2,8-Di-tert-butyl-5,11-bis(4-tert-butylphenyl)-6,12-diphenyltetracene (TBRb), Bis(2-phenylbenzothiazolato)(acetylacetonate)irdium(III) (Ir(BT)₂(acac)), Bis(2-(9,9-diethytl-fluoren-2-yl)-1-phenyl-1H-benzo[d]imdiazolato)(acetylacetonate)iridium(III) (Ir(fbi)₂(acac)), Bis(2-phenylpyridine)(3-(pyridine-2-yl)-2H-chromen-2-onate)iridium(III) (fac-Ir(ppy)₂Pc), Bis(2-(2,4-difluorophenyl)quinoline)(picolinate)iridium(III) (FPQIrpic), Bis(4-phenylthieno[3,2-c]pyridinato-N,C2′) (acetylacetonate) iridium(III) (PO-01) and/or combinations thereof.

The contents of the yellow-green host in the third layer 840C can be about 50 wt % to about 99 wt %, for example, about 80 wt % to about 95 wt %, and the contents of the yellow-green emitter in the third layer 840C can be about 1 wt % to about 50 wt %, for example, about 5 wt % to about 20 wt %, but is not limited thereto. When the third layer 840C includes both the P-type yellow-green host and the N-type yellow-green host, the P-type yellow-green host and the N-type yellow-green host can be admixed, but is not limited to, with a weight ratio of about 4:1 to about 1:4, for example about 3:1 to about 1:3.

In FIG. 6, the OLED D3 has three emitting parts, but the OLED can have two emitting parts by omitting the third emitting part 900 and the CGL2 880.

The OLED D3 has a tandem structure and includes the organic compound having the structure of Chemical Formulae 1 to 8 which has beneficial luminous properties owing to its enhanced delayed fluorescent property. The organic compound has high singlet and/or triplet energy levels and wide energy bandgap compared to the emitter. In the OLED D3 including the organic compound with beneficial affinity to holes as well as electrons, the luminous efficiency can be improved. It is possible to realize white emission with beneficial luminous efficiency and luminous lifetime in the OLED D3 that has multiple emitting parts and including the organic compound.

### Synthesis Example 1: Synthesis of Compound 1

### (1) Synthesis of Intermediate 1-1

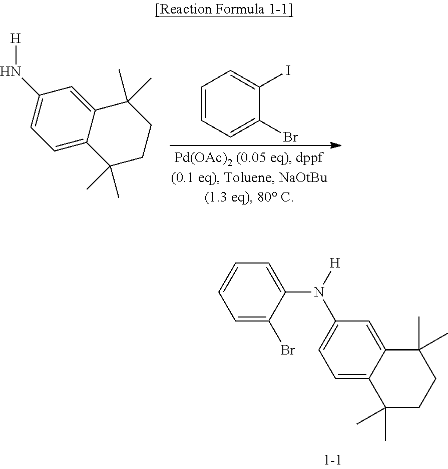 [SMILES: CC1(C)CCC(C)(C)c2cc(N)ccc21 ; Brc1ccccc1I ; CC1(C)CCC(C)(C)c2cc(Nc3ccccc3Br)ccc21]

5,5,8,8-tetramethyl-5,6,7,8-tetrahydronaphthalen-2-amine (10 g) was dissolved in toluene, then the solution was stirred under nitrogen atmosphere. 1,1′-Bis(diphenylphosphino)ferrocene (dppf, 0.1 equivalent) and sodium tert-butoxide (NaOtBu, 1.3 equivalent) were added into the solution. After 10 minutes, Palladium(II) acetate (Pd(OAc)₂, 0.05 equivalent) and 2-bromo-iodobenzene (1.05 equivalent) were added into the solution, then the solution was stirred at 80° C. for 16 hours. After the reactants were cooled to a room temperature, the reactants were extracted with ethyl acetate (EtOAc) and distilled water to obtain a crude product. The crude product was purified with a silica gel column chromatography (mobile phase: EtOAc/n-hexane (1/20)) to give an Intermediate 1-1.

### (2) Synthesis of Intermediate 1-2

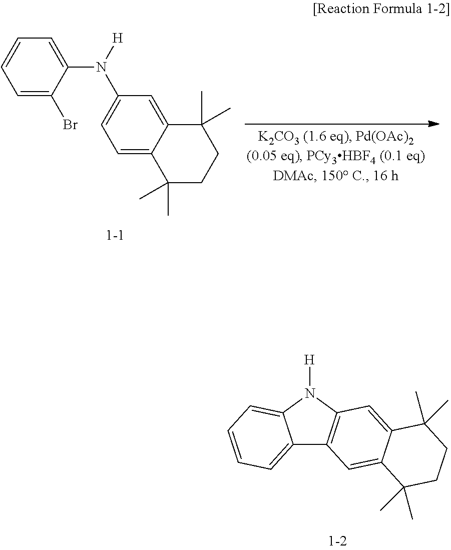 [SMILES: CC1(C)CCC(C)(C)c2cc(Nc3ccccc3Br)ccc21 ; CC1(C)CCC(C)(C)c2cc3c(cc21)[nH]c1ccccc13]

The Intermediate 1-1 (15 g) was dissolved in N,N-dimethylacetamide (DMAc), then the solution was stirred under nitrogen atmosphere. After 10 minutes, Pd(OAc)₂ (0.05 equivalent) and tricylcohexylphosphine tetrafluoroborate (Pcy3·HBF₄, 0.1 equivalent) were added into the solution, then the solution was stirred 150° C. for 16 hours to obtain a crude product. The crude product was purified with a silica gel column chromatography (mobile phase: EtOAc/n-hexane (1/20)) to give an Intermediate 1-2.

### (3) Synthesis of Intermediate 1-3

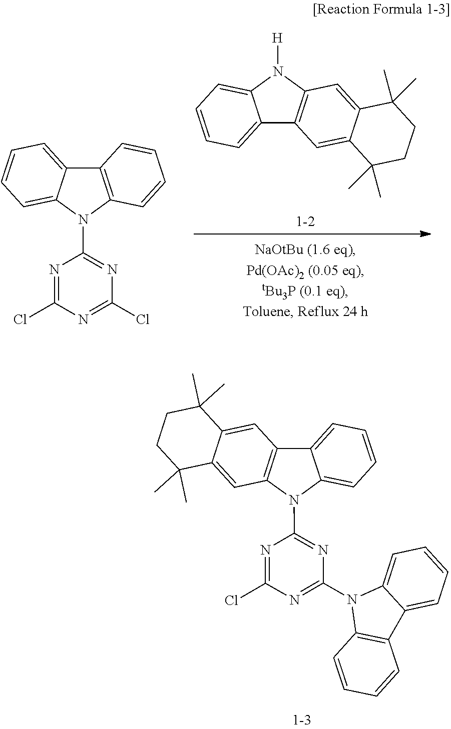 [SMILES: Clc1nc(Cl)nc(-n2c3ccccc3c3ccccc32)n1 ; CC1(C)CCC(C)(C)c2cc3c(cc21)[nH]c1ccccc13 ; CC1(C)CCC(C)(C)c2cc3c(cc21)c1ccccc1n3-c1nc(Cl)nc(-n2c3ccccc3c3ccccc32)n1]

The Intermediate 1-2 (837 mg) and 9-(4,6-dichloro-1,3,5-triazin-2-yl)-9H-carbazole (1.05 equivalent) were dissolved in tolene, the solution was stirred under nitrogen atmosphere, then NaOtBu (1.6 equivalent) was added into the solution. After 10 minutes, Pd(OAc)₂ (0.05 equivalent) and tri-tert-butlyphosphine (tBu₃P) in 50% toluene 0.1 (equivalent) were added into the solution, then the solution was stirred at 80° C. for 24 hours. After the reactants were cooled to a room temperature, the reactants were extracted with EtOAc and distilled water to obtain a crude product. The crude product was purified with a silica gel column chromatography (mobile phase: ETOAc/n-hexane (1/50)) to give an Intermediate 1-3.

### (4) Synthesis of Compound 1

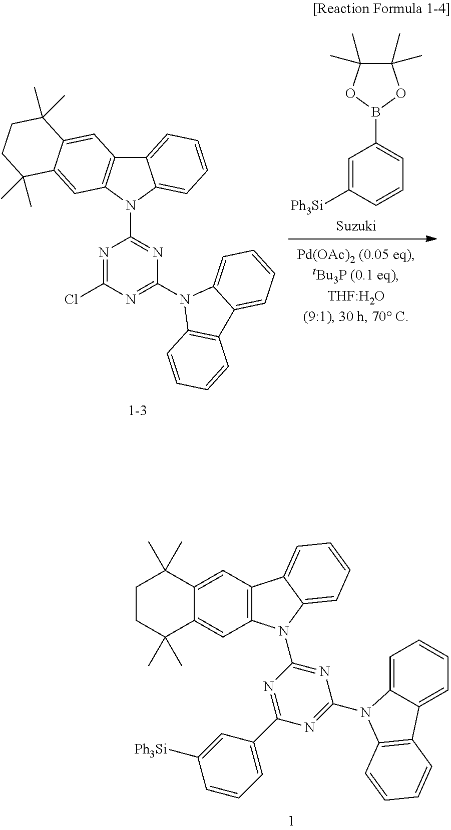

The Intermediate 1-3 (3 g) was dissolved in a mixed solvent of THF/Water (9:1), then the solution was stirred under nitrogen atmosphere. Triphenyl-(3-(4,4,5,5-tetramethyl-1,3,2-dioxaborolan-2-yl)phenyl)silane (1.05 equivalent) and K₂CO₃ (2 equivalent) were added into the solution. After 10 minutes, Pd(OAc)₂ (0.05 equivalent) and tBu₃P in 50% toluene (0.1 equivalent) were added into the solution, then the solution was stirred at 70° C. for 30 minutes to obtain a crude product. The crude product was purified with a silica gel column chromatography (mobile phase: EtOAc/n-hexane (1/50)) to give Compound 1.

### Synthesis Example 2: Synthesis of Compound 2

### (1) Synthesis of Intermediate 2-1

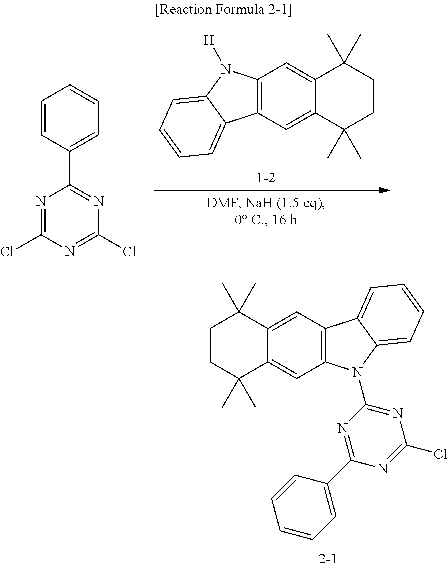 [SMILES: Clc1nc(Cl)nc(-c2ccccc2)n1 ; CC1(C)CCC(C)(C)c2cc3c(cc21)[nH]c1ccccc13 ; CC1(C)CCC(C)(C)c2cc3c(cc21)c1ccccc1n3-c1nc(Cl)nc(-c2ccccc2)n1]

2,4-dichloro-6-phenyl-1,3,5-triazine (1.05 equivalent) was dissolve in DMF. The Intermediate 1-2 (1 equivalent) and NaH (1.5 equivalent) were added into the solution, then the solution was stirred at 0° C. under nitrogen atmosphere. After 5 minutes, the reaction solution was raised to a room temperature, then the solution was stirred for 16 hours. The reactants was washed with methanol three times to give an Intermediate 2-1.

### (2) Synthesis of Compound 2

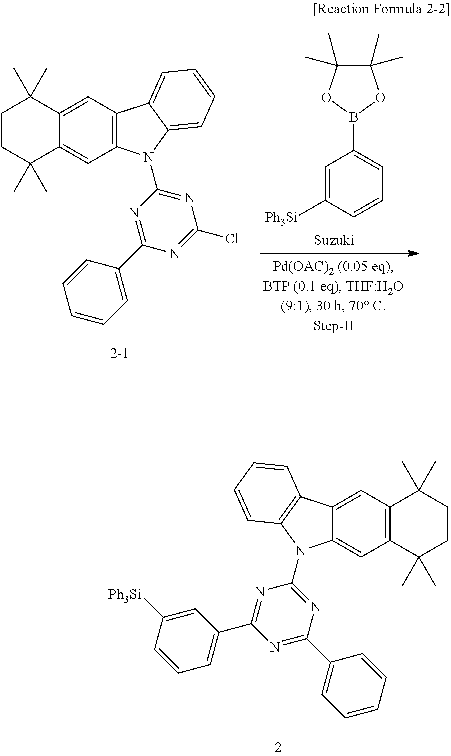

The Intermediate 2-1 (3 g) was dissolved in a mixed solvent of THF/Water (9:1), then the solution was stirred under nitrogen atmosphere. Triphenyl-(3-(4,4,5,5-tetramethyl-1,3,2-dioxaborolan-2-yl)phenyl)silane (1.05 equivalent) and K₂CO₃ (2 equivalent) were added into the solution. After 10 minutes, Pd(OAc)₂ (0.05 equivalent) and tBu₃P in 50% toluene (0.1 equivalent) were added into the solution, then the solution was stirred at 70° C. for 30 minutes to obtain a crude product. The crude product was purified with a silica gel column chromatography (mobile phase: EtOAc/n-hexane (1/50)) to give Compound 2.

### Experimental Example 1: Measurement of Emission Peak and Energy Level of Compound 1

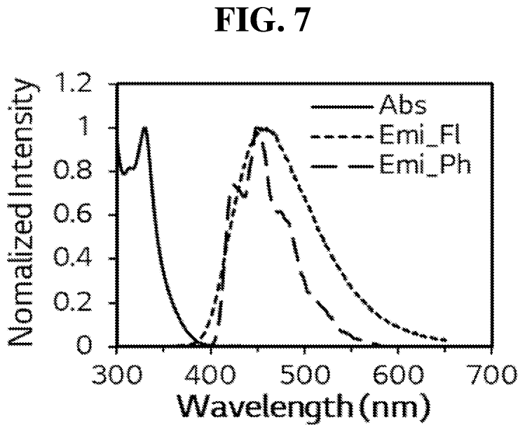

Absorption spectrum (Abs), photoluminescence spectrum, emission wavelength, singlet energy level (S₁), triplet energy level (T₁), energy bandgap (ΔE_{ST}) between singlet energy level and triplet energy level of Compound 1 were measured using GC-09 method (toluene 10-3 M). Table 1 below and FIG. 7 illustrate the measurement results.

TABLE 1
Emission Peak and Energy level of Compound 1

| Abs.ₒₙₛₑₜ | S_{1, onset} | S_{1, max} | T_{1, onset} | T_{1, max} | ΔE_{ST, onset} | ΔE_{ST, max} |
|---|---|---|---|---|---|---|
| (nm/eV) | (nm/eV) | (nm/eV) | (nm/eV) | (nm/eV) | (eV) | (eV) |
| 382.3/ | 395.8/ | 457.0/ | 406.1/ | 425.0/ | 0.08 | −0.11 |
| 3.24 | 3.13 | 2.71 | 3.05 | 2.92 | | |

### Experimental Example 1: Evaluation of Energy Level of Compounds

HOMO energy level, LUMO energy level, singlet energy level (S₁), triplet energy level (T₁), energy bandgap (ΔE_{ST}) between singlet energy level and triplet energy level of Compound 1, Compound 2, Compound 21, Compound 40 and reference compounds below were evaluated using simulation program B3LYP/6-31G(d)⁺.

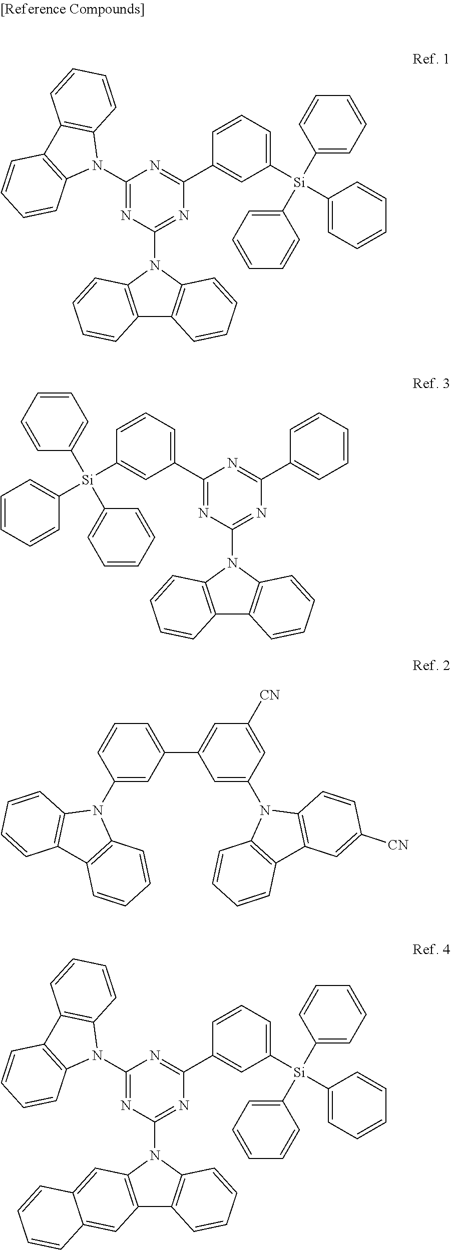 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc(-c2nc(-c3cccc([Si](c4ccccc4)(c4ccccc4)c4ccccc4)c3)nc(-n3c4ccccc4c4ccccc43)n2)cc1 ; N#Cc1cc(-c2cccc(-n3c4ccccc4c4ccccc43)c2)cc(-n2c3ccccc3c3cc(C#N)ccc32)c1 ; c1ccc([Si](c2ccccc2)(c2ccccc2)c2cccc(-c3nc(-n4c5ccccc5c5ccccc54)nc(-n4c5ccccc5c5cc6ccccc6cc54)n3)c2)cc1]

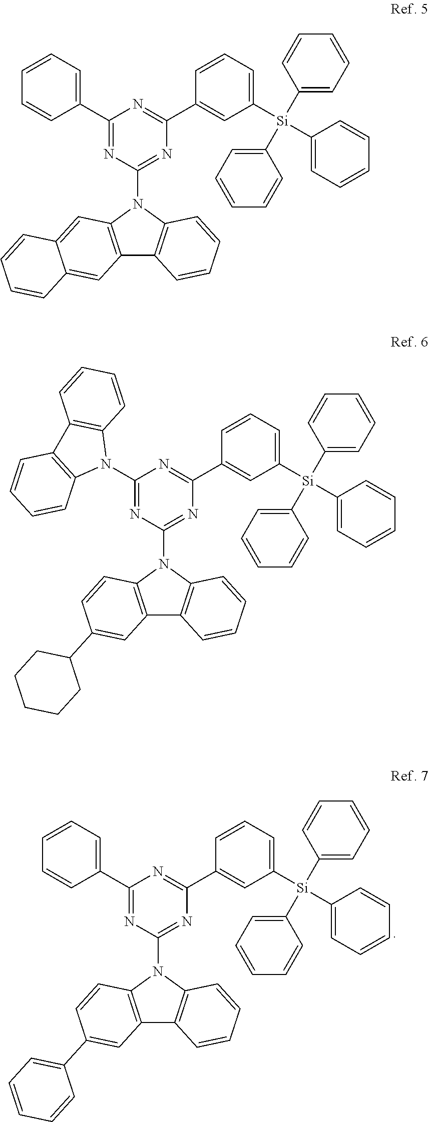 [SMILES: c1ccc(-c2nc(-c3cccc([Si](c4ccccc4)(c4ccccc4)c4ccccc4)c3)nc(-n3c4ccccc4c4cc5ccccc5cc43)n2)cc1 ; c1ccc([Si](c2ccccc2)(c2ccccc2)c2cccc(-c3nc(-n4c5ccccc5c5ccccc54)nc(-n4c5ccccc5c5cc(C6CCCCC6)ccc54)n3)c2)cc1 ; c1ccc(-c2ccc3c(c2)c2ccccc2n3-c2nc(-c3ccccc3)nc(-c3cccc([Si](c4ccccc4)(c4ccccc4)c4ccccc4)c3)n2)cc1]

Table 2 below illustrates the evaluation results and the measurement results Compound 1, Compound 2, Compound Ref.1, Compound Ref.2 and Compound Ref.3 using the same process as Experimental Example 1.

TABLE 2
Energy Level of Compounds

| | HOMO | LUMO | S₁ | T₁ | ΔEST | ΔEST |
|---|---|---|---|---|---|---|
| Compound | (eV)^{a} | (eV)^{a} | (eV)^{b} | (eV)^{b} | (eV)^{b} | (eV)^{c} |
| 1 | −5.83 | −2.59 | 3.21 | 3.06 | 0.15 | 0.08 |
| 2 | −5.88 | −2.78 | 3.05 | 3.01 | 0.04 | 0.01 |
| 21 | −5.64 | −1.76 | 3.22 | 3.05 | 0.17 | |
| 40 | −5.55 | −1.83 | 3.07 | 3.03 | 0.04 | |
| Ref. 1 | −6.00 | −2.80 | 3.42 | 3.10 | 0.32 | 0.36 |
| Ref. 2 | −6.05 | −2.69 | 3.38 | 3.10 | 0.28 | 0.35 |
| Ref. 3 | −6.11 | −3.01 | — | — | — | 0.45 |
| Ref. 4 | −5.35 | −1.65 | 3.20 | 2.42 | 0.78 | |
| Ref. 5 | −5.28 | −1.86 | 2.81 | 2.41 | 0.40 | |
| Ref. 6 | −5.64 | −1.75 | 3.37 | 3.10 | 0.27 | |
| Ref. 7 | −5.60 | −1.80 | 3.19 | 3.04 | 0.15 | |

^{a}Compound 1, Compound 2, Ref. 1, Ref. 2 and Ref. 3 was measured; other compounds were a simulation result.
^{b}simulation result
^{c}measurement

### Example 1 (Ex.1): Fabrication of OLED

An organic light emitting diode where Compound 2 was applied to an emitting material layer was fabricated. A glass substrate onto which ITO (50 nm) was coated as a thin film was washed and ultrasonically cleaned by solvent such as isopropyl alcohol, acetone and dried at 100° C. oven. The substrate was transferred to a vacuum chamber for depositing emissive layer. Subsequently, an emissive layer and a cathode were deposited by evaporation from a heating boat under about 5-7×10⁻⁷ Torr with setting a deposition rate 1 Å/s as the following order:

A hole injection layer (HIL, HAT-CN, 10 nm); a hole transport layer (HTL, NPB, 40 nm); an electron blocking layer (EBL, TAPC, 10 nm); emitting material layer (EML, Compound 1 (44 wt %), Compound PH1-9 in Chemical Formula 10 (44 wt %), Compound PD6 in Chemical Formula 22 (12 wt %), 30 nm); hole blocking layer (HBL, B3PYMPM, 10 nm); electron transport layer (ETL, TPBi, 30 nm); electron injection layer (EIL, LiF, 70 nm); and cathode (Al, 70 nm).

The structures of materials of hole injecting material, hole transporting material, electron blocking material, hole blocking material and electron transporting material are illustrated in the following:

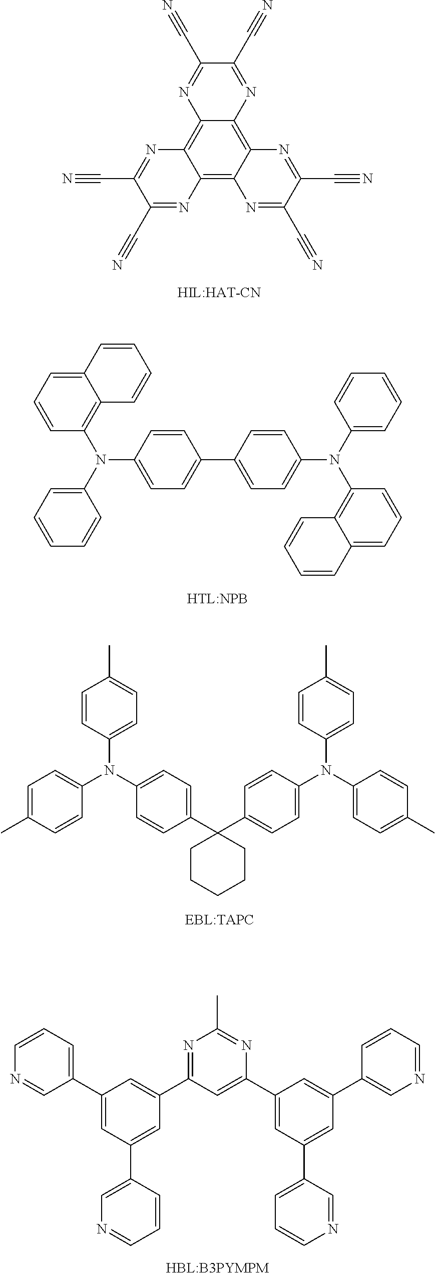

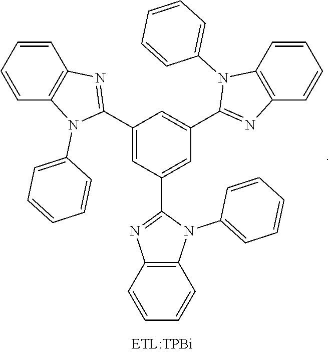 [SMILES: c1ccc(-n2c(-c3cc(-c4nc5ccccc5n4-c4ccccc4)cc(-c4nc5ccccc5n4-c4ccccc4)c3)nc3ccccc32)cc1]

### Example 2 (Ex. 2): Fabrication of OLED

An OLED was fabricated using the same procedure and the same materials as Example 1, except that Compound PH2-8 in Chemical Formula 12 instead of Compound PH1-9 was used as the P-type host in the EML.

### Example 3 (Ex. 3): Fabrication of OLED

An OLED was fabricated using the same procedure and the same materials as Example 1, except that Compound 2 instead of Compound 1 was used as the N-type host in the EML.

### Example 4 (Ex. 4): Fabrication of OLED

An OLED was fabricated using the same procedure and the same materials as Example 3, except that Compound PH2-8 in Chemical Formula 12 instead of Compound PH1-9 was used as the P-type host in the EML.

### Example 5 (Ex. 5): Fabrication of OLED

An OLED was fabricated using the same procedure and the same materials as Example 3, except that contents of the Compound 1 and the Compound PH1-9 as a host in the EML was changed to 40 wt %, respectively, and Compound TD1-2 (20 wt %) in Chemical Formula 16 of delayed fluorescent material instead of Compound PD6 was used as the emitter in the EML.

### Example 6 (Ex. 6): Fabrication of OLED

An OLED was fabricated using the same procedure and the same materials as Example 5, except that Compound PH2-8 in Chemical Formula 12 instead of Compound PH1-9 was used as the P-type host in the emitting material layer.

### Example 7 (Ex. 7): Fabrication of OLED

An OLED was fabricated using the same procedure and the same materials as Example 5, except that Compound 2 instead of Compound 1 was used as the N-type host in the EML.

### Example 8 (Ex. 8): Fabrication of OLED

An OLED was fabricated using the same procedure and the same materials as Example 7, except that Compound PH2-8 in Chemical Formula 12 instead of Compound PH1-9 was used as the P-type host in the EML.

### Example 9 (Ex. 9): Fabrication of OLED

An OLED was fabricated using the same procedure and the same materials as Example 5, except that contents of the Compound TD1-2 of the delayed fluorescent material in the EML was changed 19.5 wt %, and Compound FD1-28 (0.5 wt %) in Chemical Formula 24 of fluorescent material was added in the EML.

### Example 10 (Ex. 10): Fabrication of OLED

An OLED was fabricated using the same procedure and the same materials as Example 9, except that Compound PH2-8 in Chemical Formula 12 instead of Compound PH1-9 was used as the P-type host in the emitting material layer.

### Example 11 (Ex. 11): Fabrication of OLED

An OLED was fabricated using the same procedure and the same materials as Example 9, except that Compound 2 instead of Compound 1 was used as the N-type host in the EML.

### Example 12 (Ex. 12): Fabrication of OLED

An OLED was fabricated using the same procedure and the same materials as Example 9, except that Compound PH2-8 in Chemical Formula 12 instead of Compound PH1-9 was used as the P-type host in the EML.

### Comparative Example 1 (Ref. 1): Fabrication of OLED

An OLED was fabricated using the same procedure and the same materials as Example 1, except that Compound Ref.1 instead of Compound 1 was used as the N-type host in the EML.

### Comparative Example 2 (Ref. 2): Fabrication of OLED

An OLED was fabricated using the same procedure and the same materials as Comparative Example 1, except that Compound PH2-8 in Chemical Formula 12 instead of Compound PH1-9 was used as the P-type host in the EML.

### Comparative Example 3 (Ref. 3): Fabrication of OLED

An OLED was fabricated using the same procedure and the same materials as Example 1, except that Compound Ref.2 instead of Compound 1 was used as the N-type host in the EML.

### Comparative Example 4 (Ref. 4): Fabrication of OLED

An OLED was fabricated using the same procedure and the same materials as Comparative Example 3, except that Compound PH2-8 in Chemical Formula 12 instead of Compound PH1-9 was used as the P-type host in the EML.

### Comparative Example 5 (Ref. 5): Fabrication of OLED

An OLED was fabricated using the same procedure and the same materials as Example 1, except that Compound Ref.3 instead of Compound 1 was used as the N-type host in the EML.

### Comparative Example 6 (Ref. 6): Fabrication of OLED

An OLED was fabricated using the same procedure and the same materials as Example 5, except that Compound Ref.1 instead of Compound 1 was used as the N-type host in the EML.

### Comparative Example 7 (Ref. 7): Fabrication of OLED

An OLED was fabricated using the same procedure and the same materials as Comparative Example 6, except that Compound PH2-8 in Chemical Formula 12 instead of Compound PH1-9 was used as the P-type host in the EML.

### Comparative Example 8 (Ref. 8): Fabrication of OLED

An OLED was fabricated using the same procedure and the same materials as Example 5, except that Compound Ref.2 instead of Compound 1 was used as the N-type host in the EML.

### Comparative Example 9 (Ref. 9): Fabrication of OLED

An OLED was fabricated using the same procedure and the same materials as Comparative Example 8, except that Compound PH2-8 in Chemical Formula 12 instead of Compound PH1-9 was used as the P-type host in the EML.

### Comparative Example 10 (Ref. 10): Fabrication of OLED

An OLED was fabricated using the same procedure and the same materials as Example 5, except that Compound Ref.3 instead of Compound 1 was used as the N-type host in the EML.

### Comparative Example 11 (Ref. 11): Fabrication of OLED

An OLED was fabricated using the same procedure and the same materials as Example 9, except that Compound Ref.1 instead of Compound 1 was used as the N-type host in the EML.

### Comparative Example 12 (Ref. 12): Fabrication of OLED

An OLED was fabricated using the same procedure and the same materials as Comparative Example 11, except that Compound PH2-8 in Chemical Formula 12 instead of Compound PH1-9 was used as the P-type host in the EML.

### Comparative Example 13 (Ref. 13): Fabrication of OLED

An OLED was fabricated using the same procedure and the same materials as Example 9, except that Compound Ref.2 instead of Compound 1 was used as the N-type host in the EML.

### Comparative Example 14 (Ref. 14): Fabrication of OLED

An OLED was fabricated using the same procedure and the same materials as Comparative Example 13, except that Compound PH2-8 in Chemical Formula 12 instead of Compound PH1-9 was used as the P-type host in the EML.

### Comparative Example 15 (Ref. 15): Fabrication of OLED

An OLED was fabricated using the same procedure and the same materials as Example 9, except that Compound Ref.3 instead of Compound 1 was used as the N-type host in the EML.

### Experimental Example 3: Measurement of Luminous Properties of OLEDs

Each of the OLEDs, having 9 mm² of emission area, fabricated in Examples 1 to 12 and Comparative Examples 1 to 15 was connected to an external power source and then luminous properties for all the OLEDs were evaluated using a constant current source (KEITHLEY) and a photometer PR650 at room temperature. In particular, EQE and CIEy color coordinates for each the OLEDs were measured at a current density 8.6 mA/cm². The measurement results for the OLEDs fabricated in Examples 1 to 12 are illustrated in the following Table 3 and the measurement results for the OLEDs fabricated in Comparative Examples 1 to 15 are illustrated in the following Table 3.

TABLE 3
Luminous Properties of OLED

| | N-type | P-type | Emitter | Emitter | EQE | |
|---|---|---|---|---|---|---|
| Sample | host | host | 1 | 2 | (%) | CIEy |
| Ex. 1 | 1 | PH1-9 | PD6 | — | 16.3 | 0.168 |
| Ex. 2 | | PH2-8 | | — | 17.9 | 0.160 |
| Ex. 3 | 2 | PH1-9 | | — | 17.3 | 0.175 |
| Ex. 4 | | PH2-8 | | — | 17.2 | 0.182 |
| Ex. 5 | 1 | PH1-9 | TD1-2 | — | 22.9 | 0.341 |
| Ex. 6 | 1 | PH2-8 | | — | 23.1 | 0.321 |
| Ex. 7 | 2 | PH1-9 | | — | 23.4 | 0.346 |
| Ex. 8 | 2 | PH2-8 | | — | 23.9 | 0.339 |
| Ex. 9 | 1 | PH1-9 | | FD1-28 | 24.9 | 0.236 |
| Ex. 10 | | PH2-8 | | | 25.1 | 0.224 |
| Ex. 11 | 2 | PH1-9 | | | 26.3 | 0.239 |
| Ex. 12 | | PH2-8 | | | 25.8 | 0.232 |

TABLE 4
Luminous Properties of OLED

| | N-type | P-type | | | EQE | |
|---|---|---|---|---|---|---|
| Sample | host | host | Emitter 1 | Emitter 2 | (%) | CIEy |
| Ref. 1 | Ref-1 | PH1-9 | PD6 | — | 14.0 | 0.169 |
| Ref. 2 | Ref-1 | PH2-8 | PD6 | — | 15.4 | 0.154 |
| Ref. 3 | Ref-2 | PH1-9 | PD6 | — | 13.8 | 0.180 |
| Ref. 4 | Ref-2 | PH2-8 | PD6 | — | 14.2 | 0.172 |
| Ref. 5 | Ref-3 | PH1-9 | PD6 | — | 10.8 | 0.236 |
| Ref. 6 | Ref-1 | PH1-9 | TD1-2 | — | 20.5 | 0.337 |
| Ref. 7 | Ref-1 | PH2-8 | TD1-2 | — | 20.1 | 0.326 |
| Ref. 8 | Ref-2 | PH1-9 | TD1-2 | — | 20.7 | 0.363 |
| Ref. 9 | Ref-2 | PH2-8 | TD1-2 | — | 21.0 | 0.342 |
| Ref. 10 | Ref-3 | PH1-9 | TD1-2 | — | 17.2 | 0.370 |
| Ref. 11 | Ref-1 | PH1-9 | TD1-2 | FD1-28 | 23.6 | 0.218 |
| Ref. 12 | Ref-1 | PH2-8 | TD1-2 | FD1-28 | 22.3 | 0.211 |
| Ref. 13 | Ref-2 | PH1-9 | TD1-2 | FD1-28 | 24.2 | 0.255 |
| Ref. 14 | Ref-2 | PH2-8 | TD1-2 | FD1-28 | 22.8 | 0.243 |
| Ref. 15 | Ref-3 | PH1-9 | TD1-2 | FD1-28 | 20.1 | 0.287 |

As indicated in Tables 3 and 4, compared to the OLEDs fabricated in Comparative Examples 1-5 where phosphorescent material was used as the emitter in the EML, in the OLEDs fabricated in Examples 1-4, the EQE was improved by maximally 66.7%. Compared to the OLEDs fabricated in Comparative Examples 6-10 where delayed fluorescent material was used as the emitter in the EML, in the OLEDs fabricated in Examples 5-8, the EQE was improved by maximally 39.0%. Compared to the OLEDs fabricated in Comparative Examples 11-15 where delayed fluorescent material and fluorescent material were used as the emitter in the EML, in the OLEDs fabricated in Examples 9-12, the EQE was improved by maximally 30.8%. In addition, compared to the OLEDs fabricated in Comparative Examples 1-15, in the OLEDs fabricated in Examples 1-12, deep blue emission was realized owing to lower CIEy value.

It will be apparent to those skilled in the art that various modifications and variations may be made in the present disclosure without departing from the scope of the disclosure. Thus, it is intended that the present disclosure cover the modifications and variations of the present disclosure provided they come within the scope of the appended claims.

What is claimed is:

1. An organic compound having the following structure of Chemical Formula 1: 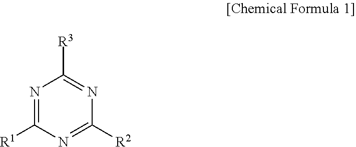
wherein, in the Chemical Formula 1,
each of R¹, R² and R³ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least one of R¹, R² and R³ has the following moiety of Chemical Formula 2 and at least one of R¹, R² and R³ has the following moiety of Chemical Formula 3: 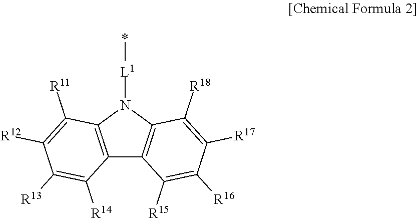
wherein, in the Chemical Formula 2,
each of R¹¹, R¹², R¹³, R¹⁴, R¹⁵, R¹⁶, R¹⁷, and R¹⁸ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least two groups among R¹¹, R¹², R¹³, R¹⁴, R¹⁵, R¹⁶, R¹⁷, and R¹⁸ are linked together to form an unsubstituted or substituted C₅-C₂₀ alicyclic ring or an unsubstituted or substituted C₃-C₂₀ hetero alicyclic ring; and
L¹ is a single bond, an unsubstituted or substituted C₆-C₃₀ arylene group or an unsubstituted or substituted C₃-C₃₀ hetero arylene group, 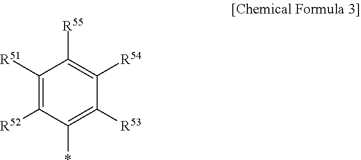
wherein, in the Chemical Formula 3,
each of R⁵¹, R⁵², R⁵³, R⁵⁴ and R⁵⁵ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

2. The organic compound of claim 1, wherein the moiety of Chemical Formula 2 has the following structure of Chemical Formula 4A, Chemical Formula 4B, Chemical Formula 4C, Chemical Formula 4D or Chemical Formula 4E: 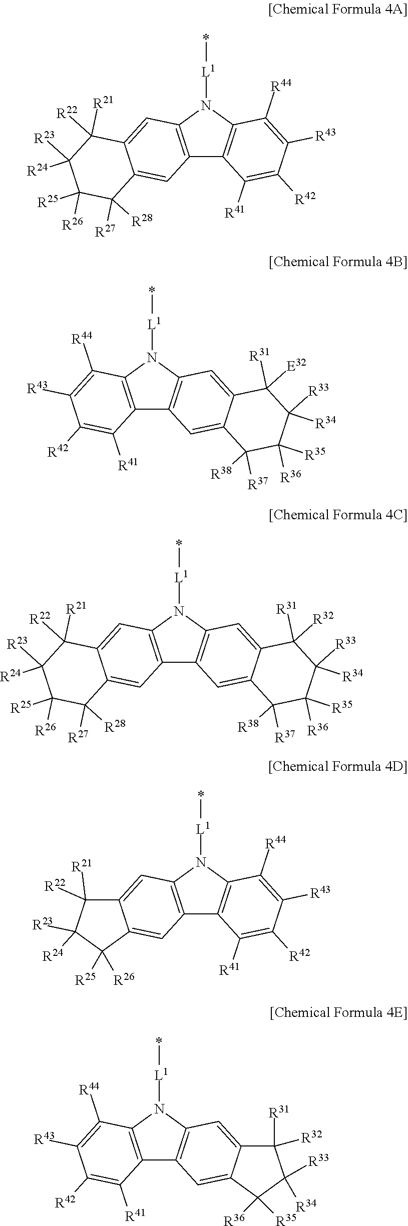
wherein, in the Chemical Formulae 4A to 4E,
L¹ is a same as defined in Chemical Formula 2;
each of R²¹, R²², R²³, R²⁴, R²⁵, R²⁶, R²⁷, R²⁸, R³¹, R³², R³³, R³⁴, R³⁵, R³⁶, R³⁷ and R³⁸ is independently hydrogen or an unsubstituted or substituted C₁-C₂₀ alkyl group; and
each of R⁴¹, R⁴², R⁴³ and R⁴⁴ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

3. The organic compound of claim 2, wherein each of R⁴¹, R⁴², R⁴³ and R⁴⁴ in Chemical Formulae 4A to 4E is independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

4. The organic compound of claim 1, wherein the moiety of Chemical Formula 2 has the following structure of Chemical Formula 5A, Chemical Formula 5B, or Chemical Formula 5C: 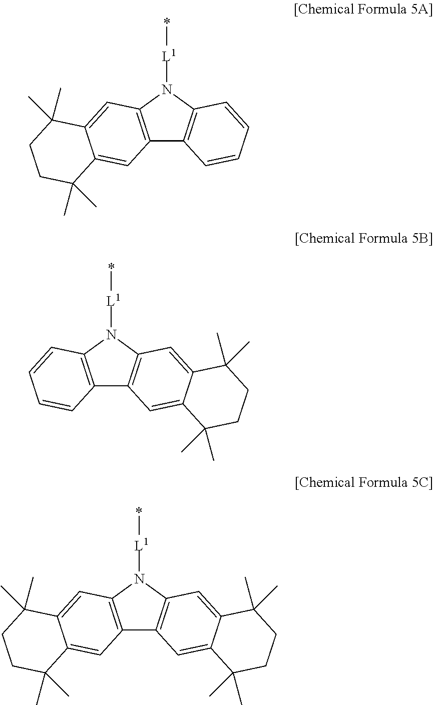
wherein, in the Chemical Formulae 5A, 5B and 5C,
L¹ is a same as defined in Chemical Formula 2.

5. The organic compound of claim 1, wherein the moiety of Chemical Formula 2 has the following structure of Chemical Formula 6A, Chemical Formula 6B, Chemical Formula 6C, Chemical Formula 6D, Chemical Formula 6E or Chemical Formula 6F: 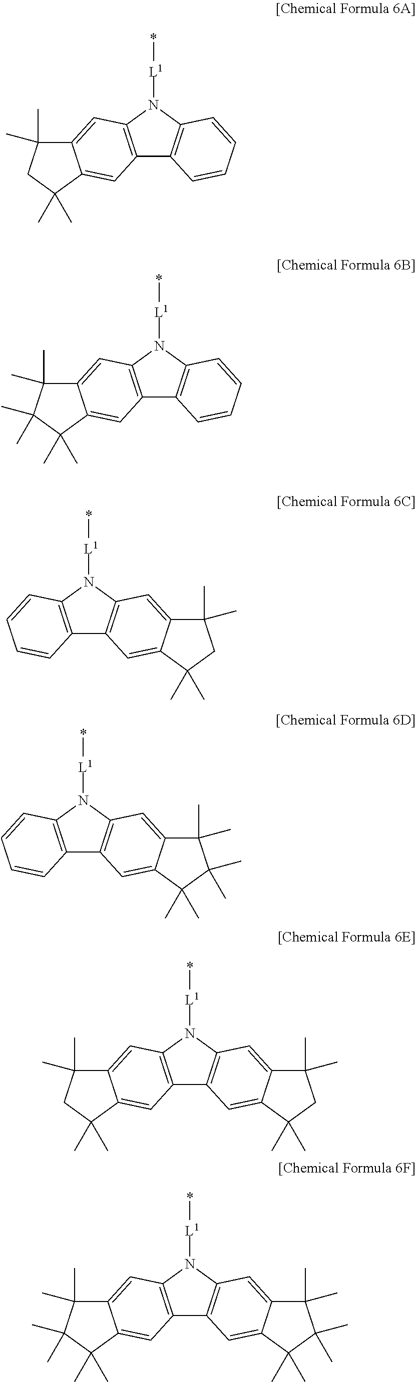
wherein, in the Chemical Formulae 6A to 6F,
L¹ is a same as defined in Chemical Formula 2.

6. The organic compound of claim 1, wherein the moiety of Chemical Formula 3 has the following structure of Chemical Formula 7A, Chemical Formula 7B, or Chemical Formula 7C: 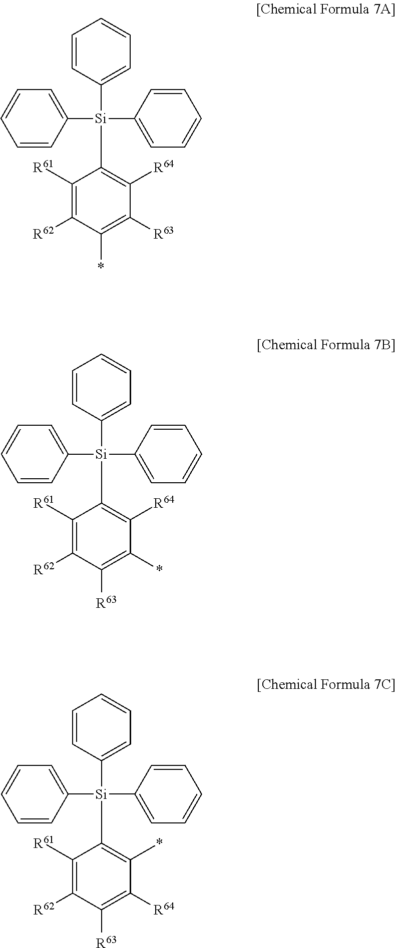
wherein, in the Chemical Formula 7A to 7C,
each of R⁶¹, R⁶², R⁶³, and is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

7. The organic compound of claim 6, wherein each of R⁶¹, R⁶², R⁶³, and R⁶⁴ in Chemical Formulae 7A to 7C is independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

8. The organic compound of claim 1, wherein each of R¹, R⁵², R⁵³, R⁵⁴ and R⁵⁵ in the Chemical Formula 3 is independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

9. The organic compound of claim 1, wherein the organic compound is at least one of the following compounds:

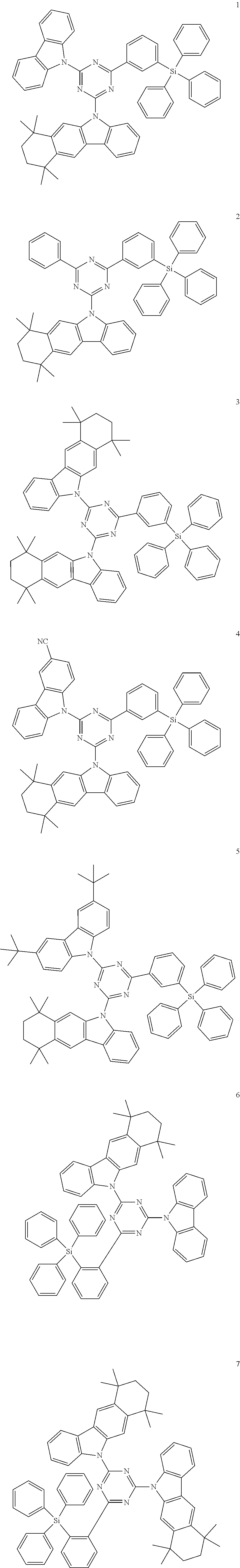

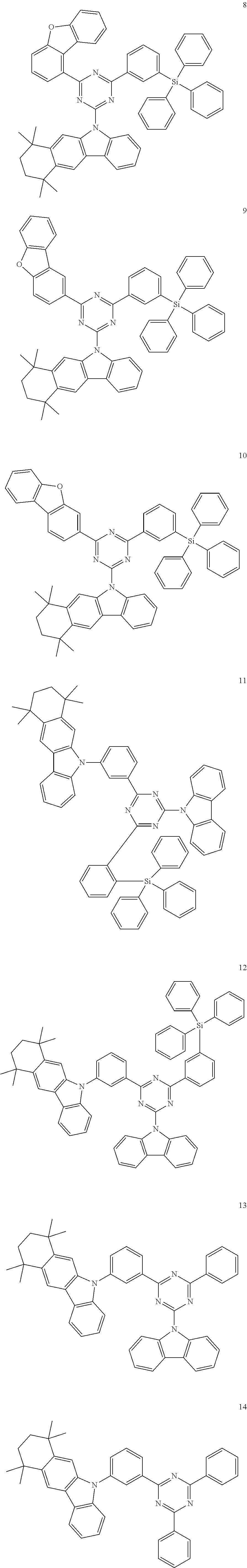

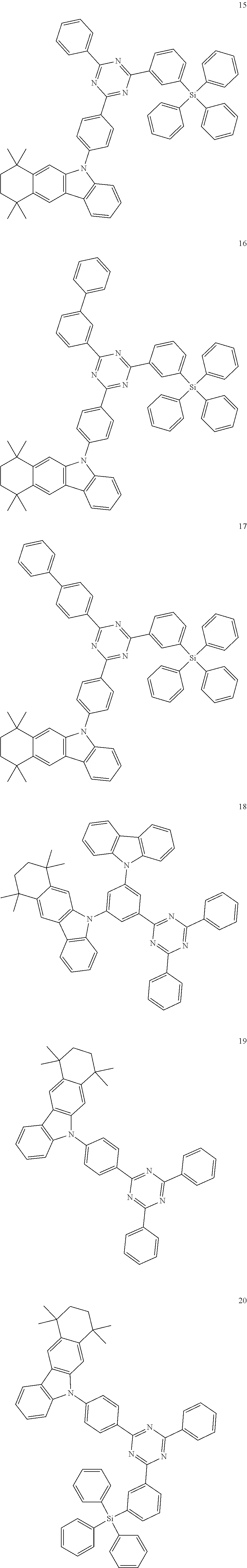

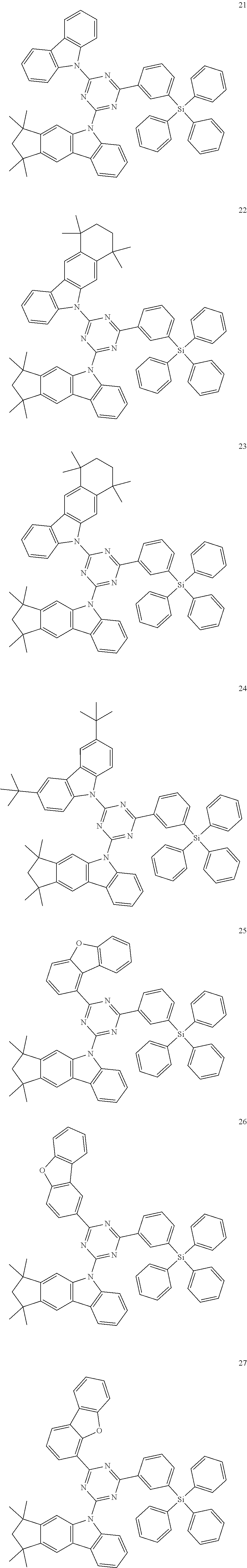

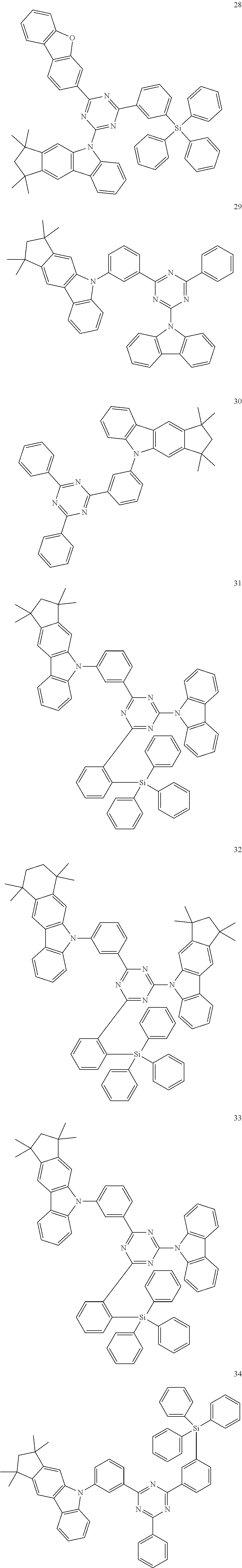

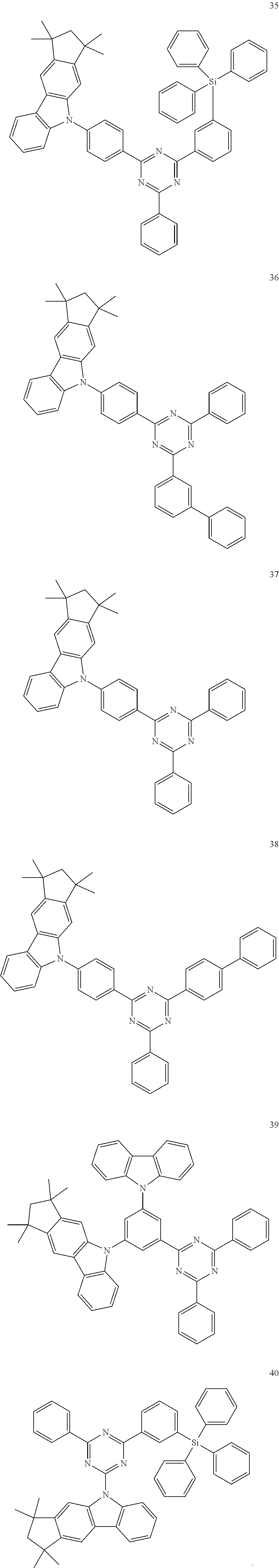

10. An organic light emitting diode, including:
a first electrode;
a second electrode facing the first electrode; and
an emissive layer disposed between the first electrode and the second electrode,
wherein the emissive layer includes an organic compound having the following structure of Chemical Formula 1: 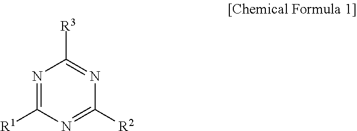
wherein, in the Chemical Formula 1,
each of R¹, R² and R³ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least one of R¹, R² and R³ has the following moiety of Chemical Formula 2 and at least one of R¹, R² and R³ has the following moiety of Chemical Formula 3: 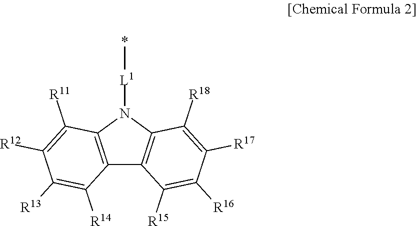
wherein, in the Chemical Formula 2,
each of R¹¹, R¹², R¹³, R¹⁴, R¹⁵, R¹⁶, R¹⁷, and R¹⁸ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group, where at least two groups among R¹¹, R₁₂, R¹³, R¹⁴, R¹⁵, R¹⁶, R¹⁷, and R¹⁸ are linked together to form an unsubstituted or substituted C₅-C₂₀ alicyclic ring or an unsubstituted or substituted C₃-C₂₀ hetero alicyclic ring; and
L¹ is a single bond, an unsubstituted or substituted C₆-C₃₀ arylene group or an unsubstituted or substituted C₃-C₃₀ hetero arylene group, 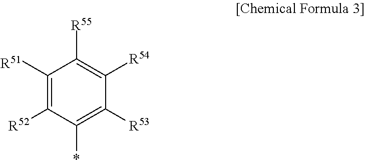
wherein, in the Chemical Formula 3,
each of R⁵¹, R⁵², R⁵³, R⁵⁴ and R⁵⁵ is independently hydrogen, an unsubstituted or substituted C₁-C₂₀ alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

11. The organic light emitting diode of claim 10, wherein the moiety of Chemical Formula 2 has the following structure of Chemical Formula 4A, Chemical Formula 4B, Chemical Formula 4C, Chemical Formula 4D or Chemical Formula 4E: 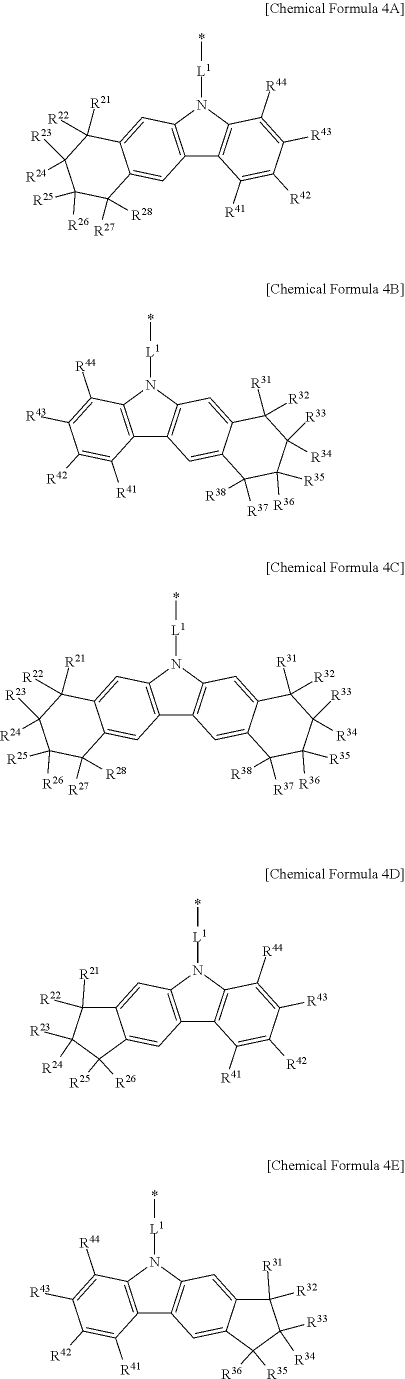
wherein, in the Chemical Formulae 4A to 4E,
L¹ is a same as defined in Chemical Formula 2;
each of R²¹, R²², R²³, R²⁴, R²⁵, R²⁶, R²⁷, R²⁸, R³¹, R³², R³³, R³⁴, R³⁵, R³⁶, R³⁷ and R³⁸ is independently hydrogen or an unsubstituted or substituted C₁-C₂₀ alkyl group; and
each of R⁴¹, R⁴², R⁴³ and R⁴⁴ is independently hydrogen, an unsubstituted or substituted C₁-C₂₀alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

12. The organic light emitting diode of claim 11, wherein each of R⁴¹, R⁴², R⁴³ and R⁴⁴ in Chemical Formulae 4A to 4E is independently hydrogen, an unsubstituted or substituted C₁-C₂₀alkyl group, an unsubstituted or substituted C₆-C₃₀ aryl group or an unsubstituted or substituted C₃-C₃₀ hetero aryl group.

13. The organic light emitting diode of claim 10, wherein the moiety of Chemical Formula 2 has the following structure of Chemical Formula 5A, Chemical Formula 5B or Chemical Formula 5C: 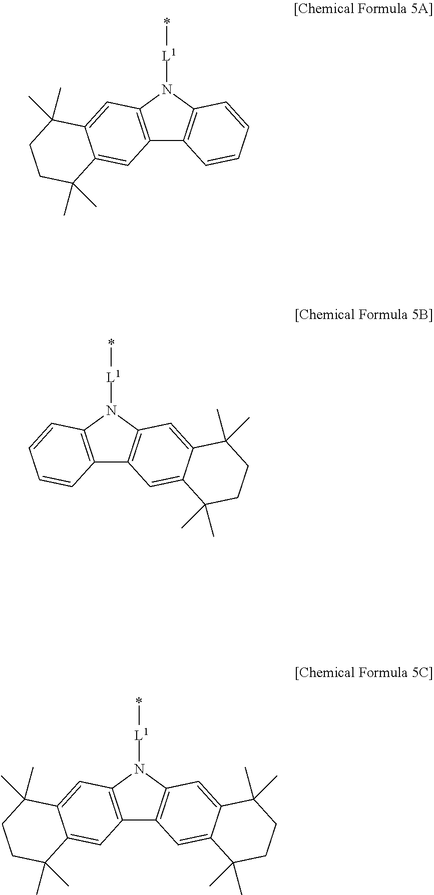
wherein, in the Chemical Formulae 5A, 5B and 5C,
L¹ is a same as defined in Chemical Formula 2.

14. The organic light emitting diode of claim 10, wherein the moiety of Chemical Formula 2 has the following structure of Chemical Formula 6A, Chemical Formula 6B, Chemical Formula 6C, Chemical Formula 6D, Chemical Formula 6E or Chemical Formula 6F: 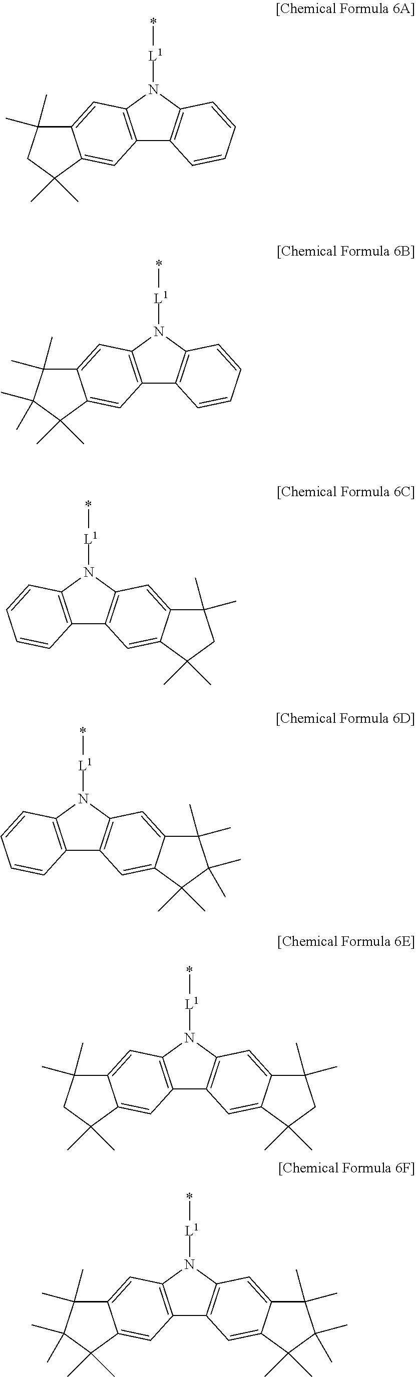
wherein, in the Chemical Formulae 6A to 6F,
L¹ is a same as defined in Chemical Formula 2.

15. The organic light emitting diode of claim 10, wherein the moiety of Chemical Formula 3 has the following structure of Chemical Formula 7A, Chemical Formula 7B, or Chemical Formula 7C: 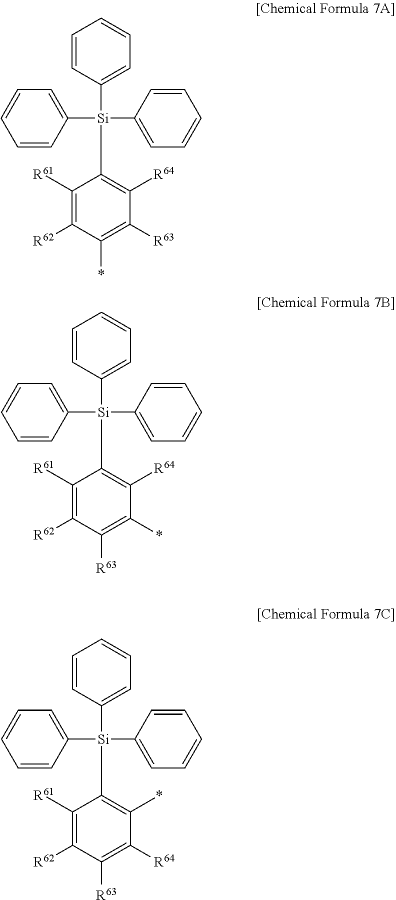
wherein, in the Chemical Formula 7A to 7C,
each of R⁶¹, R⁶², R⁶³, and R⁶⁴ is independently hydrogen, an unsubstituted or substituted C₁-C₂₀alkyl group, an unsubstituted or substituted C₁-C₂₀ alkoxy group, an unsubstituted or substituted C₁-C₂₀ alkyl silyl group, an unsubstituted or substituted C₁-C₂₀ alkyl germanyl group, an unsubstituted or substituted C₁-C₂₀ alkyl amino group, an unsubstituted or substituted C₁-C₂₀ alkyl thio group, an unsubstituted or substituted C₆-C₃₀ aryl group, an unsubstituted or substituted C₃-C₃₀ hetero aryl group, an unsubstituted or substituted C₆-C₃₀ aryl oxy group, an unsubstituted or substituted C₃-C₃₀ hetero aryl oxy group, an unsubstituted or substituted C₆-C₃₀ aryl silyl group, an unsubstituted or substituted C₃-C₃₀ hetero aryl silyl group, an unsubstituted or substituted C₆-C₃₀ aryl germanyl group, an unsubstituted or substituted C₃-C₃₀ hetero aryl germanyl group, an unsubstituted or substituted C₆-C₃₀ aryl amino group, an unsubstituted or substituted C₃-C₃₀ hetero aryl amino group, an unsubstituted or substituted C₆-C₃₀ aryl thio group or an unsubstituted or substituted C₃-C₃₀ hetero aryl thio group.

16. The organic light emitting diode of claim 15, wherein each of R⁶¹, R⁶², R⁶³, and R⁶⁴ in Chemical Formulae 7A to 7C is independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

17. The organic light emitting diode of claim 10, wherein each of R⁵¹, R⁵², R⁵³, R⁵⁴ and R⁵⁵ in the Chemical Formula 3 is independently hydrogen, an unsubstituted or substituted C₁-C₂₀ alkyl group, an unsubstituted or substituted C₆-C₃₀ aryl group or an unsubstituted or substituted C₃-C₃₀ hetero aryl group.

18. The organic light emitting diode of claim 10, wherein the emissive layer includes at least one emitting material layer.

19. The organic light emitting diode of claim 18, wherein the at least one emitting material layer includes a first host and wherein the first host includes the organic compound.

20. The organic light emitting diode of claim 19, wherein the at least one emitting material layer further includes a second host.

21. The organic light emitting diode of claim 19, wherein the at least one emitting material layer further includes at least one emitter.

22. The organic light emitting diode of claim 21, wherein the at least one emitter includes at least one of a phosphorescent emitter, a fluorescent emitter, and a delayed fluorescent emitter.

23. The organic light emitting diode of claim 21, wherein the at least one emitter emits blue color light.

24. The organic light emitting diode of claim 18, wherein the emissive layer further includes a hole injection layer, a hole transport layer, an electron blocking layer, the at least one emitting material layer, a hole blocking layer, an electron transport layer, an electron injection layer, and/or a charge generation layer.

25. The organic light emitting diode of claim 10, wherein the emissive layer has a single emitting unit.

26. The organic light emitting diode of claim 10, wherein the emissive layer includes:
a first emitting part disposed between the first electrode and the second electrode, and including a first emitting material layer;
a second emitting part disposed between the first emitting part and the second electrode, and including a second emitting material layer; and
a first charge generation layer disposed between the first emitting part and the second emitting part, and
wherein at least one of the first emitting material layer and the second emitting material layer includes the organic compound.

27. The organic light emitting diode of claim 26, wherein the emissive layer further includes:
a third emitting part disposed between the second emitting part and the second electrode; and a second charge generation layer disposed between the second emitting part and the third emitting part.

28. An organic light emitting device, including:
a substrate; and
the organic light emitting diode of claim 10 over the substrate.
